(19) United States
(12) Patent Application Publication
(10) Pub. No.: US 2022/0178954 A1
(43) Pub. Date: Jun. 9, 2022

(54) STRATIFICATION BY SEX AND APOE GENOTYPE IDENTIFIES METABOLIC HETEROGENEITY IN ALZHEIMER'S DISEASE

(71) Applicant: DUKE UNIVERSITY, Durham, NC (US)

(72) Inventors: Gabi Kastenmüller, Oberschleißheim (DE); Rima F. Kaddurah-Daouk, Belmont, MA (US); Matthias Arnold, Oberschleißheim (DE)

(21) Appl. No.: 17/437,122

(22) PCT Filed: Mar. 6, 2020
(86) PCT No.: PCT/US2020/021399

§ 371 Date: Sep. 17, 2020

Related U.S. Application Data
(60) Provisional application No. 62/815,956, filed on Mar. 8, 2019.
(60) Provisional application No. 62/818,655, filed on Mar. 14, 2019.

(51) Int. Cl. G01N 33/92; G01N 33/68; G16H 10/60

(52) U.S. Cl. CPC G01N 33/92; G01N 33/6896; G01N 2405/06; G01N 2800/2821; G01N 2405/08; G16H 10/60

(57) ABSTRACT

Described herein are methods for stratifying Alzheimer's disease among male and female subjects by analyzing biomarker metabolites. In one aspect, the biomarker metabolite comprises one or more of PC ae C44:4, PC ac C44:5, or PA ae C44:6; or PC ac C44:4, PC ac C44:5, PC aa C32:1, PC aa C32:0, or PC ae C42:4.

### CROSS-REFERENCE TO RELATED APPLICATIONS

This application claims priority to U.S. Provisional Patent Application No. 62/815,956, filed on Mar. 8, 2019, and U.S. Provisional Patent Application No. 62/818,655, filed on Mar. 14, 2019, each of which is incorporated by reference here in in its entirety.

### FEDERALLY SPONSORED RESEARCH

This invention was made with United States government support under National Institutes of Health/National Institute on Aging grant numbers RF1 AG058942 and R01 AG057452. The United States government has certain rights in the invention.

### TECHNICAL FIELD

Described herein are methods for stratifying Alzheimer's disease among male and female subjects by analyzing biomarker metabolites. In one aspect, the biomarker metabolite comprises one or more of PC ae C44:4, PC ae C44:5, or PA ae C44:6; or PC ae C44:4, PC ae C44:5, PC aa C32:1, PC aa C32:0, or PC ae C42:4.

### BACKGROUND

Female sex has long been regarded a major risk factor for Alzheimer's disease (AD). It is assumed that out of 5.3 million people in the United States who were diagnosed with AD at age 65 or older, more than 60% are women. Also, estimates indicate that the lifetime risk of developing AD at age 45 may be almost double in females than in males [1, 2]. However, the exact role and magnitude of sexual dimorphism in predisposition and progression to AD are controversial [3-6]. While age is the strongest risk factor for late-onset AD (LOAD), the higher life expectancy of women only partially explains the observed sex difference in frequency and lifetime risk [7]. Complexity is added by several genetic studies showing a significant sex difference in effects of the APOE ε4 genotype, the strongest common genetic risk factor for LOAD. These studies report risk estimates for E4 carriers being higher in females than in males, a finding that seems to be additionally dependent on age [8-13]. APOE ε4 has also been described to be associated with AD biomarkers in a sex-dependent way with again larger risk estimates for women than for men [9, 14-16], although these findings have not been fully consistent across studies [16, 17]. Additionally, studies have suggested that sex differences in AD may change during the trajectory of disease [18], with overall risk for mild cognitive impairment (MCI), the prodromal stage of AD, being higher in males [19, 20], while progression to AD occurs at a faster rate in females, at least partly in APOE ε4-dependent ways [3, 8, 10, 18, 21, 22]. The mechanisms underlying this sex-linked and partly intertwined APOE ε4- and age-dependent heterogeneity in AD susceptibility and severity are only beginning to unravel, calling for novel approaches to further elucidate molecular sex differences in AD risk and biomarker profiles.

Interestingly, all three of the aforementioned major AD risk factors, i.e., age, APOE ε4 genotype, and sex, have a profound impact on metabolism [23-29], supporting the view of AD as a metabolic disease [30-32]. In recent years, availability of high-throughput metabolomics techniques, which can measure hundreds of small biochemical molecules (metabolites) simultaneously, allows for the study of metabolic imprints of age, genetic variation, and sex very broadly, covering the entire metabolism: (i) Age-dependent differences were observed in levels of phosphatidylcholines (PCs), sphingomyelins (SMs), acylcarnitines, ceramides, and amino acids [28, 33]. A panel of 22 independent metabolites explained 59% of the total variance in chronological age in a large twin population cohort. In addition, one of these metabolites, C-glycosyltryptophan, was associated with age-related traits including bone mineral density, lung [29] and kidney function [34]. (ii) As expected from APOE's known role in cholesterol and lipid metabolism [35, 36], common genetic variants in this gene were associated with blood cholesterol levels in genome- and metabolome-wide association studies [36, 37]. In addition, associations with levels of various SMs were identified [38, 39]. (iii) Analogous to age, sex also affects blood levels of many metabolites from a broad range of biochemical pathways. In a healthy elderly population with mostly post-menopausal women, females showed higher levels of most lipids except lyso-PCs, while the levels of most amino acids including branched chain amino acids (BCAAs) were higher in males with the exception of glycine and serine, which were higher in women [23, 24]. In addition to studies investigating the impact of age and sex on metabolism separately, Gonzalez-Covarrubias et al. recently reported sex-specific lipid signatures associated with longevity in the Leiden Longevity Study [28]. In women, higher levels of ether-PC and SM species were associated with longevity; in men no significant differences were observed. Thus, based on results from large-scale metabolomics studies, aging may influence a wider range of metabolites in women than men, highlighting the need for sex-stratified analyses.

Many of the metabolites affected by female sex, age, and APOE genotype such as BCAAs, glutamate, and various lipids appear to be altered in AD independent of these risk factors [38, 40, 41]. In patients with MCI, alterations in lipid metabolism, lysine metabolism, and the tricarboxylic acid cycle have been observed [42, 43]. In one of the largest blood-based metabolomics studies of AD, we identified metabolic alterations in various stages across the trajectory of the disease. For instance, higher levels of SMs and PCs were observed in early stages of AD as defined by abnormal CSF Aβ₁₋₂ levels, whereas intermediate changes, measured by CSF total tau, were correlated with increased levels of SMs and long-chain acylcarnitines [44]. Changes in brain volume and cognition, usually noted in later stages, were correlated with a shift in energy substrate utilization from fatty acids to amino acids, especially BCAAs. Other metabolomics studies have reported metabolic alterations in AD which support these findings, including alterations in PCs in AD [43, 45-47] and sphingolipid transport and fatty acid metabolism in MCI/AD compared to cognitively normal (CN) subjects [48]. Higher blood concentrations of sphingolipid species were associated with disease progression and pathological severity at autopsy [49]. Metabolomics analysis of brain and blood tissue further revealed that bile acids, important regulators of lipid metabolism and products of human-gut microbiome co-metabolism, were altered in AD [50, 51] and associated with brain glucose metabolism and atrophy as well as CSF Aβ₁₋₄₂ and p-tau [52]. In most of these studies, sex as well as APOE ε4 genotype, were used as covariates. Thus, sex-specific associations between AD and metabolite levels or associations that are modified by sex with opposite effect directions for the two sexes might have been missed in these analyses. Similarly, sex-by-APOE genotype interactions would have been masked.

What is needed are methods of stratifying Alzheimer's disease among male and female subjects in male and female subjects by analyzing biomarker metabolites in comparison to control subjects.

### SUMMARY

One embodiment described herein is a method for stratifying Alzheimer's disease among male and female subjects, the method comprising: the method comprising: determining in a sample from the subject the level of at least one biomarker metabolite selected from the group consisting of PC ae C44:4, PC ae C44:5, PA ae C44:6, PC aa C32:1, PC aa C32:0, and PC ae C42:4; and diagnosing the subject as having Alzheimer's disease or an increased risk of Alzheimer's disease when the level of the at least one biomarker metabolite in the sample from the subject is different from or greater than the level in a control. In one aspect, the biomarker metabolites are selected from PC ae C44:4, PC ae C44:5, and PA ae C44:6. In another aspect, the biomarker metabolites are selected from PC ae C44:4, PC ae C44:5, PC aa C32:1, PC aa C32:0, and PC ae C42:4.

Another embodiment described herein is a method of diagnosing or detecting Alzheimer's disease in a male subject, the method comprising: determining in a sample from the subject the level of at least one biomarker metabolite selected from the group consisting of PC ae C32:1, threonine, PC ae C36:1, PC ae C36:2, asparagine, glycine, one hydroxy-SM (SM (OH) C16:1), PC ae C40:2, and C16:1; and diagnosing the subject as having Alzheimer's disease or an increased risk of Alzheimer's disease when the level of the at least one biomarker metabolite in the sample from the subject is different from or greater than the level in a control. In one aspect, the biomarker metabolites comprise PC ae C32:1. In another aspect, the biomarker metabolites comprise threonine. In another aspect, the biomarker metabolites are selected from PC ae C36:1, PC ae C36:2, asparagine, glycine, and one hydroxy-SM (SM (OH) C16:1). In another aspect, the biomarker metabolites are selected from PC ae C40:2 and C16:1. In another aspect, the biomarker metabolites comprise C16:1.

Another embodiment described herein is a method of diagnosing or detecting Alzheimer's disease in a female subject, the method comprising: determining in a sample from the subject the level of at least one biomarker metabolite selected from the group consisting of C5-DC (C6-OH), C8, C10, C2, valine, proline, and histidine; and diagnosing the subject as having Alzheimer's disease or an increased risk of Alzheimer's disease when the level of the at least one biomarker metabolite in the sample from the subject is different from or greater than the level in a control. In one aspect, the biomarker metabolites comprise valine. In another aspect, the biomarker metabolites are selected from C5-DC (C6-OH), C8, C10, C2, and histidine. In another aspect, the biomarker metabolites comprise proline. In another aspect, the biomarker metabolites comprise C10.

Another embodiment described herein is a method of diagnosing or detecting Alzheimer's disease in an APOE ε4 carrier subject, the method comprising: determining in a sample from the subject the level of at least one biomarker metabolite selected from the group consisting of PC ae C44:6, PC ae C44:4, PC ae C44:5, and PC ae C42:4; and diagnosing the subject as having Alzheimer's disease or an increased risk of Alzheimer's disease when the level of the at least one biomarker metabolite in the sample from the subject is different from or greater than the level in a control.

Another embodiment described herein is a method of diagnosing or detecting Alzheimer's disease in an APOE ε4 non-carrier subject, the method comprising: determining in a sample from the subject the level of the biomarker metabolite C10; and diagnosing the subject as having Alzheimer's disease or an increased risk of Alzheimer's disease when the level of the biomarker metabolite in the sample from the subject is different from or greater than the level in a control.

Another embodiment described herein is a method of diagnosing or detecting Alzheimer's disease in a female APOE ϑ4 carrier subject, the method comprising: determining in a sample from the subject the level of at least one biomarker metabolite selected from PC ae C42:4, PC ae C44:5, PC e C44:6, C10, and proline; and diagnosing the subject as having Alzheimer's disease or an increased risk of Alzheimer's disease when the level of the at least one biomarker metabolite in the sample from the subject is different from or greater than the level in a control. In one aspect, the sample from the subject comprises whole blood, serum, plasma, or cerebral spinal fluid (CSF). In another aspect, the method further comprising administering to the subject a treatment for Alzheimer's disease. In another aspect, the control sample is taken from a subject or population of subjects with normal cognition.

### DESCRIPTION OF THE DRAWINGS

FIG. 1. Study rationale. A. This study aims to investigate the relationship between AD, sex, and metabolic readouts in a systematic fashion. The background of this work are: first, it has been reported that AD risk may be increased in females; second, there are strongly pronounced, highly significant, and often replicated sex differences in metabolite concentrations in the general, healthy population; and, third, results have shown that there are significant associations of metabolite levels with AD and its biomarkers. In this study, we examined: (i) if clinical diagnosis of LMCI or AD influences metabolic sex differences as seen in healthy controls; (ii) by performing stratified analyses by sex coupled with effect heterogeneity estimation, if sex modulates associations of metabolite levels with three AD biomarkers across the A-T-N spectrum; and, (iii), as the APOE ε4 genotype is associated with metabolite concentration changes, it further is a strong risk factor for late-onset AD, and some reports have stated a sex difference in risk predisposition exerted by APOE4 status, we finally checked if effects of metabolites showing sex-based effect heterogeneity in their associations with AD are also modulated by APOE4 status. Finally, we performed two-fold stratification by both sex and APOE4 status in order to identify potential interactions between the two variables. B. To address the three research questions of this study, we first performed analyses of sex-metabolite associations for 139 metabolites in the ADNI cohort stratified by diagnostic group (question i). Subsequently, we performed phenotype (A-T-N)-metabolite associations for 139 metabolites in the ADNI cohort stratified by sex (question ii) and stratified by APOE ε4 status; additionally, we performed phenotype (A-T-N)-metabolite associations for the 21 significantly associated metabolites after stratification by sex plus APOE ε4 status (question iii).

FIG. 2. Scatter plots showing Z-scores of effect estimates of metabolite associations with A-T-N biomarkers for males (x-axis) versus those for females (y-axis). Homogeneous effects (i.e., those with same effect direction and comparable effect size) are located close to the diagonal, heterogeneous effects are located close to the anti-diagonal, and sex-specific effects are located close to one axis, i.e., x-axis for male-specific and y-axis for female-specific effects, respectively. Homogeneous, overall significant results are depicted as diamonds, effects with significant heterogeneity are drawn as rectangles, and effects significant in only one sex are displayed as triangles. Metabolites additionally marked by an asterisk are significant in one sex only and simultaneously show significant heterogeneity. Sex-specificity is further illustrated by a color scale (blue: females; green: males). On the upper right panel, example boxplots of metabolite residuals (obtained by regressing out included covariates) for each effect type are shown separately for females and males with (in dark red) and without (in light red) CSF Aβ₁₋₄₂ pathology, respectively.

FIG. 3. Boxplots showing residuals of proline levels (derived by regressing out covariate effects) for A: the full sample; B: 1-fold stratifications by sex; C: 1-fold stratification by APOE4 status; and D: 2-fold stratification by both sex and APOE4 status; separately for high (light blue) and low (darker blue; derived by mean-split) FDG-PET values. The only subgroup showing a significant difference in proline levels are APOE4+ females with substantially higher levels in subjects with lower brain glucose uptake

FIG. 4. Metabolic sex differences in the ADNI cohorts. We tested whether sex-associated differences in blood metabolite levels differ between patients with probable AD, subjects with LMCI, and CN subjects in the ADNI cohorts. We found 108 of 140 metabolites to be significantly associated with sex after multiple testing correction while adjusting for age, BMI, ADNI study phase, and diagnostic group. 70 of these associations replicate previous findings in a healthy population using. All SMs and the majority of PCs were more abundant in women. The majority of biogenic amines, amino acids, and acylcarnitines were more abundant in men. Stratifying subjects by diagnostic group revealed that 53 of the 108 metabolites showing significant sex-differences were also significant in each of the three groups (AD, LMCI, CN) alone, while 14 metabolites showed no significant difference in any of the groups, probably due to lower statistical power after stratification. No significant sex-differences were found that were not also significant in the unstratified analysis.

FIGS. 5A-5U. Boxplots for 21 metabolites identified in relation to A-T-N biomarkers in 2-fold stratified analyses. Boxplots are shown for. 5A: asparagine; 5B: C10; SC: C16:1; 5D: C2; 5E: C5-DC (C6-OH); 5F: C8; 5G: glycine; 5H: histidine; 5I: PC aa C32:0; 5J: PC aa C32:1; 5K: PC ae C36:1; 5L: PC ae C36:2; SM: PC ae C40:2; 5N: PC ae C42:4; 5O: PC ae C44:4; SP: PC ae C44:5; 5Q: PC ae C44:6; 5R: proline; SS: SM (OH) C16:1; 5T: threonine; and SU: valine for 2-fold stratified analyses by both sex and APOE4 status (A: pathological CSF Aβ₁₋₄₂; T: mean-split CSF p-tau levels; N: mean-split FDG-PET values). APOE4 status groups are plotted in separate panels, females and males are distinguished by color (f: blue, m: green), and binarized biomarker groups are emphasized by lighter (lower-risk biomarker profile) and deeper (higher-risk biomarker profile) colors.

FIG. 6 Quotient normalization for batch removal exemplified for PC ae C44:5. The A. shows boxplots of log 2-transformed levels of PC ae C44:5 in study samples for all batches (defined by 96-well plates) prior to quotient normalization. Differences between plates as well as between runs for ADNI-1 (batches 1-11) vs. ADNI-GO/2 (batches 12-23) are clearly identifiable. The B. shows log 2-transformed levels of PC ae C44:5 in study samples for all batches after quotient normalization using measurements for NIST standards. The shift between ADNI-1 and -GO/2 is not significant any more, however, differences between single batches are still observable. As measurements for NIST standards are assumed to be stable across plates, the remaining variability should be biological variance due to random distribution of study samples across plates. To ascertain that this is the case, i.e. that technical confounds are removed but true biological variance is retained, we perform additional QC steps using blinded duplicates/triplicates of study samples distributed across batches and remove metabolites that show excessive values for the coefficient of variation and the infraclass correlation coefficient. The C. shows the log 2-transformed (for clearer visualization) correction factors. These correction or dilution factors are obtained for each batch and metabolite by dividing the global, cross-batch mean concentration of a metabolite in NIST standards by the within-batch mean concentration in NIST standards of the same metabolite. Metabolite concentrations in study samples are then multiplied by the factor derived for the batch a study sample was contained in.

FIG. 7. Estimation and significance of effect heterogeneity exemplified by the association of C2 with CSF p-tau. A. Histograms of the distribution of log 2-transformed CSF p-tau levels in all samples, and for females and males separately. B. Histograms of the distribution of log 2-transformed, Z-scored C2 levels in all samples, and for females and males separately. C. Scatter plots of C2 levels versus CSF p-tau levels plus regression lines. In all samples, there is absolutely no effect, in females there is a nominally significant correlation, and in males, there is a non-significant opposite trend (i.e. negative correlation). D. Density plots of the estimated effects in all samples, females, and males.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Further, unless otherwise required by context, singular terms shall include pluralities and plural terms shall include the singular.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

As used herein, the terms “treat”, “treating,” or “treatment” of any disease or disorder refer In an embodiment, to ameliorating the disease or disorder (i.e., slowing or arresting or reducing the development of the disease or at least one of the clinical symptoms thereof). In an embodiment, “treat,” “treating,” or “treatment” refers to alleviating or ameliorating at least one physical parameter including those which may not be discernible by the patient.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

The term “prophylaxis” refers to preventing or reducing the progression of a disorder, either to a statistically significant degree or to a degree detectable to one skilled in the art.

The term “substantially” as used herein means to a great or significant extent, but not completely.

As used herein, all percentages (%) refer to mass (or weight, w/w) percent unless noted otherwise.

The term “about” as used herein refers to any values, including both integers and fractional components that are within a variation of up to ±10% of the value modified by the term “about.” As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

Terms such as “include,” “including,” “contain,” “containing,” “having,” and the like mean “comprising.”

The term “or” can be conjunctive or disjunctive.

Here, we examine the role of sex in the relationship between metabolic alterations and AD, in order to elucidate possible metabolic underpinnings for the observed sexual dimorphism in AD susceptibility and severity. Using metabolomics data from 1,517 subjects of the Alzheimer's Disease Neuroimaging Initiative (ADNI) cohorts, we specifically investigate how sex modifies the associations of representative A-T-N biomarkers [53, 54] (A: CSF Aβ₁₋₄₂ pathology; T: CSF p-tau; N: region of interest (ROI)-based glucose uptake measured by FDG-PET) with 140 blood metabolites by stratified analyses and systematic comparison of effects between men and women. In downstream analyses, we then inspect sex-differences in metabolic effects on AD biomarkers for dependencies on APOE genotype, both by interaction analysis and sub-stratification.

Embodiments described herein relate generally to the analysis and identification of global metabolic changes in Alzheimer's disease (AD). More particularly, materials and methods relating to the use of metabolomics as a biochemical approach to identify peripheral metabolic changes in AD patients and correlate them to cerebrospinal fluid pathology markers, imaging features, and cognitive performance are described herein.

Baseline serum samples were profiled from the Alzheimer's Disease Neuroimaging Initiative-1 (ADNI-1) cohort where vast data exist on each patient including cognitive decline and imaging changes over many years, information on CSF markers, genetics, and other “-omics” data. CSF biomarkers were used to define early metabolic changes in cognitively normal participants who have CSF pathology and to evaluate metabolic signatures that might be related to Aβ₁₋₄₂ and tau pathology. Using partial correlation networks, progressive metabolic changes were defined that accompany changes in CSF Aβ₁₋₄₂, CSF tau, brain structure, and cognition, whereas coexpression networks were used to connect key metabolic changes implicated in disease. The relationship of metabolites with longitudinal cognitive and imaging changes helped us define metabolic signatures correlated with disease progression. Key associations were also present in multiple independent cohorts. The systems approach described in the present disclosure facilitated the elucidation of metabolic changes along different stages during the progression of AD and led to the identification of valuable peripheral biomarkers that can inform and accelerate clinical trials.

The biochemical information about disease mechanisms that can be used as a roadmap for novel drug discovery and establishment of blood-based biomarkers. Eight complementary, targeted and non-targeted, metabolomics platforms are currently in the process of generating data on ADNI participants to define the metabolic trajectory of disease connecting central and peripheral metabolic failures in a pathway and network context. The present disclosure expands on biochemical coverage to better understand disease pathogenesis by using complementary data unique to ADNI-1. The unique opportunity of having longitudinal cognitive and imaging data on each subject for close to a decade enables identification of peripheral biomarkers that are disease related.

Accordingly, the present disclosure demonstrates the use of a targeted, highly validated metabolomics platform with the analysis guided by CSF markers and imaging data. Using 1,517 base-line serum samples from the ADNI-1 cohort, relationships between metabolomics data and cross-sectional clinical, CSF, and MRI measures were systematically evaluated, as well as their association with longitudinal cognitive and brain volume changes. Multiple comparisons and covariate-adjusted analyses, that included relevant medications, identified sets of metabolites that became altered at specific disease stages (preclinical AD with biomarker-defined AD pathology vs. symptomatic stages). Using partial correlation networks, the results of the present disclosure integrates data on the metabolic effects on AD pathogenesis, linking central and peripheral metabolism in a way that consistently addresses biochemical trajectories of disease with this established temporal sequence of pathophysiological stages of AD.

### Aβ Pathology

Embodiments described herein identified changes in biomarker metabolites in early AD subjects, including biomarkers defined preclinical stages in CN participants, which were present in higher concentrations as compared to controls. These included a specific set of PCs (e.g., PC ae C36.2, PC ae C40.3, PC ae C42.4, and PC ae C44.4) and SMs (SM (OH) C14.1, SM C16.0). These biomarker metabolites were associated with abnormal CSF Aβ₁₋₄₂ values in CN subjects to a similar degree as observed in MCI subjects, indicating an early role of ether-containing PC species and SM in the development of Alzheimer's disease. In some cases, these metabolites were also associated with later cognitive decline and global brain atrophy changes in the MCI group. The data of indicate imbalances and/or dysfunction with phospholipid metabolism in early phases of Alzheimer's disease progression. Partial correlation networks showed that the pathological CSF Aβ₁₋₄₂ values were associated with two groups of lipids, composed primarily of ether-containing PCs and relatively short-chain SMs. Ether-containing PC (PC ae) biomarker metabolites are PC species with an ether linkage of an aliphatic chain to the first hydroxyl position of glycerol. These lipids may represent a mixture of lipid metabolites including but not limited to, plasmalogens, acyl-alkyl PC, or PC containing an odd-numbered fatty acyl chain. When measured in a biological sample such as serum, for example, ether-containing lipids are derived from liver metabolism and are possible indicators of peroxisomal function and lipid oxidation status. Plasmalogens and SMs may be enriched in membrane rafts where they facilitate signal transduction and serve as a source for lipid secondary messengers. The association of PCs and SMs described in the present disclosure with early changes in AD and with pathological CSF Aβ₁₋₄₂ levels may be indicative of early neurodegeneration and loss of membrane function. Ether-linked PC biomarker metabolites may be found in high abundance in plasma membranes and are a source for signaling molecules, including platelet-activating factor and arachidonic acid. Similarly, they may be found in high abundance in immune cells, are regulatory factors, and may be part of a link between inflammation and AD. Both SMs and ether-linked PCs may be located in membrane rafts, suggesting that lipid rafts are directly associated with regulation of amyloid precursor protein processing, the production of Aβ₁₋₄₂, and facilitate its aggregation.

### Tau Pathology

In accordance with embodiments of the present disclosure, pathological CSF Aβ₁₋₄₂ shows an association with ether-linked PCs, and shorter chain SMs, but not amines, lysoPC, or acylcarnitines. Aβ₁₋₄₂ changes happen early in Alzheimer's disease, followed by accumulation of tau protein in the CSF. As described herein, tau-related biomarker metabolites were different both from those that correlate with Aβ₁₋₄₂ as well as from metabolites associated with brain atrophy and cognitive changes. Tau-related metabolites may belong to an intermediate stage between Aβ₁₋₄₂ accumulation and changes in imaging and cognitive function, further demonstrating that different metabolic events occur at different disease stages. For example long-chain acylcarnitines, PC ae C36:2, and SM.C20:2 were present in higher concentrations in cognitively impaired subjects, as compared to controls, with AD-like CSF Aβ₁₋₄₂ values, indicating that changes in these metabolites are more specific to AD-related neurodegeneration. Additionally, accumulation of acylcarnitine species containing long fatty acyl chains indicates malfunction of fatty acid transport and/or β-oxidation in mitochondria, inefficient utilization of fatty acids as energy substrates, and/or alterations in tau metabolism. Levels of several acylcarnitine species were increased either at the MCI stage or in clinical AD.

The present disclosure provides the material and methods pertaining to the use of metabolomics and network approaches to identify lipid metabolic changes related to early stages of AD, as well as later changes related to mitochondrial energetics and energy utilization. The lipid changes identified herein reflect alterations in membrane structure and function early in the disease process and suggest a change in lipid rafts, which in turn, cause alterations in Aβ processing. Over time, the changes in lipid membranes, particularly mitochondrial membranes, may result in increased lipid oxidation, loss of membrane potential, and changes in membrane transport. In some cases, lipid membrane changes might involve disruptions in BCAA as an energy source, production of acylcarnitines, and altered energy substrate utilization.

Amino acids are the monomeric building blocks of proteins, which in turn comprise a wide range of biological compounds, including enzymes, antibodies, hormones, transport molecules for ions and small molecules, collagen, and muscle tissues. Amino acids are considered hydrophobic or hydrophilic, based upon their solubility in water, and, more particularly, on the polarities of their side chains. Amino acids having polar side chains are hydrophilic, while amino acids having nonpolar side chains are hydrophobic. The solubilities of amino acids, impart, determines the structures of proteins. Hydrophilic amino acids tend to make up the surfaces of proteins while hydrophobic amino acids tend to make up the water-insoluble interior portions of proteins. Of the common 20 amino acids, nine are considered essential in humans, as the body cannot synthesize them. Rather, these nine amino acids are obtained through an individual's diet. A deficiency of one or more amino acids can cause various imbalances and can lead to the development of a disease condition(s). Additionally, as described herein, the presence or absence of one or more amino acids can indicate metabolic imbalances reflective of disease conditions, such as Alzheimer's disease. Branched chain amino acids (BCAAs), which include valine, leucine, and isoleucine, are among a subgroup of amino acids that can be predictive of the development of Alzheimer's disease. As such, BCAAs can be used to treat such conditions as they have been shown to function not only as protein building blocks, but also as inducers of signal transduction pathways that modulate translation initiation.

In some cases, several ether-linked PC metabolites have been associated with a risk of diabetes; insulin resistance may promote aminoacidemia and the use of amino acids for energy, and BCAA and a-AAA have been identified as predictors of diabetes risk. BCAAs (e.g., valine, leucine, and isoleucine) are important for balanced metabolism and have been implicated in insulin resistance, type-2 diabetes mellitus, and obesity. As described herein, low levels of valine and its correlation with cognitive changes were demonstrated, pointing to an important role for this BCAA in cognitive changes in AD. Low levels of BCAAs have been implicated in hepatic insulin resistance in liver disease and may have a broader role in insulin resistance in the brain.

In some embodiments, it may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of biomarker as a function of assay units (e.g., fluorescent signal intensity, biochemical indicator). Using samples taken from multiple donors, standard curves can be provided for reference levels of a biomarker metabolite in subjects with normal cognition, for example, as well as for “at-risk” levels of the biomarker metabolite (e.g., MCI subjects) in samples obtained from donors, who may have one or more of the characteristics set forth above.

One embodiment described herein is a method of stratifying Alzheimer's disease among male and female subjects, the method comprising: determining in a sample from the subject the level of at least one biomarker metabolite selected from the group consisting of PC ae C44:4, PC ae C44:5, PA ae C44:6, PC aa C32:1, PC aa C32:0, and PC ae C42:4; and diagnosing the subject as having Alzheimer's disease or an increased risk of Alzheimer's disease when the level of the at least one biomarker metabolite in the sample from the subject is different from or greater than the level in a control. In one aspect, the biomarker metabolites are selected from PC ae C44:4, PC ae C44:5, and PA ae C44:6. In another aspect, the biomarker metabolites are selected from PC ae C44:4, PC ae C44:5, PC aa C32:1, PC aa C32:0, and PC ae C42:4. The method comprises assaying a test sample and/or a control sample for a biomarker metabolite using an assay, for example, designed to detect the metabolite itself (e.g., detectable label) and/or using an assay that compares a signal generated by a detectable label as a direct or indirect indication of the presence, amount, or concentration of a biomarker metabolite in the test sample to a signal generated as a direct or indirect indication of the presence, amount, or concentration of a control.

Another embodiment described herein is a method of diagnosing or detecting Alzheimer's disease in a male subject, the method comprising: determining in a sample from the subject the level of at least one biomarker metabolite selected from the group consisting of PC ae C32:1, threonine, PC ae C36:1, PC ae C36:2, asparagine, glycine, one hydroxy-SM (SM (OH) C16:1), PC ae C40:2, and C16:1; and diagnosing the subject as having Alzheimer's disease or an increased risk of Alzheimer's disease when the level of the at least one biomarker metabolite in the sample from the subject is different from or greater than the level in a control. In one aspect, the biomarker metabolites comprise PC ae C32:1. In another aspect, the biomarker metabolites comprise threonine. In another aspect, the biomarker metabolites are selected from PC ae C36:1, PC ae C36:2, asparagine, glycine, and one hydroxy-SM (SM (OH) C16:1). In another aspect, the biomarker metabolites are selected from PC ae C40:2 and C16:1. In another aspect, the biomarker metabolites comprise C16:1.

Another embodiment described herein is a method of diagnosing or detecting Alzheimer's disease in a female subject, the method comprising: determining in a sample from the subject the level of at least one biomarker metabolite selected from the group consisting of C5-DC (C6-OH), C8, C10, C2, valine, proline, and histidine; and diagnosing the subject as having Alzheimer's disease or an increased risk of Alzheimer's disease when the level of the at least one biomarker metabolite in the sample from the subject is different from or greater than the level in a control. In one aspect, the biomarker metabolites comprise valine. In another aspect, the biomarker metabolites are selected from C5-DC (C6-OH), C8, C10, C2, and histidine. In another aspect, the biomarker metabolites comprise proline. In another aspect, the biomarker metabolites comprise C10.

Another embodiment described herein is a method of diagnosing or detecting Alzheimer's disease in an APOE ε4 carrier subject, the method comprising: determining in a sample from the subject the level of at least one biomarker metabolite selected from the group consisting of PC ae C44:6, PC ae C44:4, PC ae C44:5, and PC ae C42:4; and diagnosing the subject as having Alzheimer's disease or an increased risk of Alzheimer's disease when the level of the at least one biomarker metabolite in the sample from the subject is different from or greater than the level in a control.

Another embodiment described herein is a method of diagnosing or detecting Alzheimer's disease in an APOE ε4 non-carrier subject, the method comprising: determining in a sample from the subject the level of the biomarker metabolite C10; and diagnosing the subject as having Alzheimer's disease or an increased risk of Alzheimer's disease when the level of the biomarker metabolite in the sample from the subject is different from or greater than the level in a control.

Another embodiment described herein is a method of diagnosing or detecting Alzheimer's disease in a female APOE ε4 carrier subject, the method comprising: determining in a sample from the subject the level of at least one biomarker metabolite selected from PC ae C42:4, PC ae C44:5, PC ae C44:6, C10, and proline; and diagnosing the subject as having Alzheimer's disease or an increased risk of Alzheimer's disease when the level of the at least one biomarker metabolite in the sample from the subject is different from or greater than the level in a control. In one aspect, the sample from the subject comprises whole blood, serum, plasma, or cerebral spinal fluid (CSF). In another aspect, the method further comprising administering to the subject a treatment for Alzheimer's disease. In another aspect, the control sample is taken from a subject or population of subjects with normal cognition.

Another embodiment described herein provides a method for diagnosing or detecting Alzheimer's disease in a subject. In accordance with these embodiments, the method includes obtaining a sample from a subject (e.g., serum sample) and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or any combinations thereof. Detecting the biomarker metabolite can then be used to establish an association with the subject having at least one independent indicator of Alzheimer's disease, such that the subject is diagnosed with having Alzheimer's disease if at least one biomarker metabolite is detected. The method may also include administering a treatment to alleviate one or more symptoms of AD and may also include assessing the biomarker metabolite again in order to determine if the treatment is therapeutically beneficial.

In some embodiments, the present disclosure provides methods for diagnosing or detecting Mild Cognitive Impairment (MCI) in a subject, and/or distinguishing between early phases of AD from late states of AD. In accordance with these embodiments, the method includes obtaining a sample from a subject (e.g., serum sample) and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or combinations thereof. Detecting the biomarker metabolite can then be used to establish an association with the subject having at least one independent indicator of MCI. The method may also include administering a treatment to alleviate one or more symptoms of MCI and may also include assessing the biomarker metabolite again in order to determine if the treatment is therapeutically beneficial.

In still other embodiments, the present disclosure provides a method for predicting the outcome of a subject suspected having AD. In accordance with these embodiments, the method includes obtaining a sample from a subject and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or combinations thereof. The method may also include assessing at least one independent indicator of AD in the subject, such that detection of the at least one biomarker metabolite is associated with the subject having at least one independent indicator of AD. In some cases, the subject is predicted to develop AD if at least one biomarker metabolite is detected. The method may also include administering a treatment to alleviate one or more symptoms of MCI and may also include assessing the biomarker metabolite again in order to determine if the treatment is therapeutically beneficial.

As described and used herein, “sample,” “test sample,” and “biological sample” refer to fluid sample containing or suspected of containing a biomarker metabolite. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing a biomarker metabolite may be assayed directly. In one embodiment, the source containing a biomarker metabolite is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### REFERENCES

[1] Association As. 2017 Alzheimer's disease facts and figures. Alzheimer's & Dementia: The Journal of the Alzheimer's Association. 2017; 13:325-73.
[2] Hebert L E, Weuve J, Scherr P A, Evans D A. Alzheimer disease in the United States (2010-2050) estimated using the 2010 census. Neurology. 2013; 80:1778-83.
[3] Mielke M M, Vemuri P, Rocca W A. Clinical epidemiology of Alzheimer's disease: assessing sex and gender differences. Clinical Epidemiology. 2014; 6:37-48.
[4] Prince M, Ali G C, Guerchet M, Prina A M, Albanese E, Wu Y T. Recent global trends in the prevalence and incidence of dementia, and survival with dementia. Alzheimers Res Ther. 2016; 8:23.
[5] Fiest K M, Roberts J I, Maxwell C J, Hogan D B, Smith E E, Frolkis A, et al. The Prevalence and Incidence of Dementia Due to Alzheimer's Disease: a Systematic Review and Meta-Analysis. Can J Neurol Sci. 2016; 43 Suppl 1:S51-82.
[6] Matthews F E, Stephan B C, Robinson L, Jagger C, Bames L E, Arthur A, et al. A two decade dementia incidence comparison from the Cognitive Function and Ageing Studies I and II. Nat Commun. 2016; 7:11398.
[7] Nebel R A, Aggarwal N T, Bames L L, Gallagher A, Goldstein J M, Kantarci K, et al. Understanding the impact of sex and gender in Alzheimer's disease: A call to action. Alzheimer's & dementia: the journal of the Alzheimer's Association. 2018; 14:1171-83.
[8] Mortensen E L, Hogh P. A gender difference in the association between APOE genotype and age-related cognitive decline. Neurology. 2001; 57:89-95.
[9] Altmann A, Tian L, Henderson V W, Greicius M D, Alzheimer's Disease Neuroimaging Initiative I. Sex modifies the APOE-related risk of developing Alzheimer disease. Ann Neurol. 2014; 75:563-73.
[10] Beydoun M A, Boueiz A, Abougergi M S, Kitner-Triolo M H, Beydoun H A, Resnick S M, et al. Sex differences in the association of the apolipoprotein E epsilon 4 allele with incidence of dementia, cognitive impairment, and decline. Neurobiol Aging. 2012; 33:720-31 e4.
[11] Corder E H, Ghebremedhin E, Taylor M G, Thal D R, Ohm T G, Braak H. The biphasic relationship between regional brain senile plaque and neurofibrillary tangle distributions: modification by age, sex, and APOE polymorphism. Annals of the New York Academy of Sciences. 2004; 1019:24-8.
[12] Mahley R W, Huang Y. Apolipoprotein e sets the stage: response to injury triggers neuropathology. Neuron. 2012; 76:871-85.
[13] Neu S C, Pa J, Kukull W, Beekly D, Kuzma A, Gangadharan P, et al. Apolipoprotein E Genotype and Sex Risk Factors for Alzheimer Disease: A Meta-analysis. JAMA neurology. 2017; 74:1178-89.
[14] Damoiseaux J S, Seeley W W, Zhou J, Shirer W R, Coppola G, Karydas A, et al. Gender modulates the APOE epsilon4 effect in healthy older adults: convergent evidence from functional brain connectivity and spinal fluid tau levels. The Journal of neuroscience: the official journal of the Society for Neuroscience. 2012; 32:8254-62.
[15] Heise V, Filippini N, Trachtenberg A J, Suri S, Ebmeier K P, Mackay C E. Apolipoprotein E genotype, gender and age modulate connectivity of the hippocampus in healthy adults. Neuroimage. 2014; 98:23-30.
[16] Sampedro F, Vilaplana E, de Leon M J, Alcolea D, Pegueroles J, Montal V, et al. APOE-by-sex interactions on brain structure and metabolism in healthy elderly controls. Oncotarget. 2015; 6:26663-74.
[17] Jack C R, Jr., Wiste H J, Weigand S D, Knopman D S, Vemuri P, Mielke M M, et al. Age, Sex, and APOE epsilon4 Effects on Memory, Brain Structure, and beta-Amyloid Across the Adult Life Span. JAMA neurology. 2015; 72:511-9.
[18] Sundermann E E, Tran M, Maki P M, Bondi M W. Sex differences in the association between apolipoprotein E epsilon4 allele and Alzheimer's disease markers. Alzheimers Dement (Amst). 2018; 10:438-47.
[19] Caracciolo B, Palmer K, Monastero R, Winblad B, Backman L, Fratiglioni L. Occurrence of cognitive impairment and dementia in the community: a 9-year-long prospective study. Neurology. 2008; 70:1778-85.
[20] Roberts R O, Geda Y E, Knopman D S, Cha R H, Pankratz V S, Boeve B F, et al. The incidence of MCI differs by subtype and is higher in men: the Mayo Clinic Study of Aging. Neurology. 2012; 78:342-51.
[21] Hyman B T, Gomez-Isla T, Briggs M, Chung H, Nichols S, Kohout F, et al. Apolipoprotein E and cognitive change in an elderly population. Ann Neurol. 1996; 40:55-66.
[22] Buckley R F, Mormino E C, Amariglio R E, Properzi M J, Rabin J S, Lim Y Y, et al. Sex, amyloid, and APOE epsilon4 and risk of cognitive decline in preclinical Alzheimer's disease: Findings from three well-characterized cohorts. Alzheimer's & dementia: the journal of the Alzheimer's Association. 2018; 14:1193-203.
[23] Krumsiek J, Mittelstrass K, Do K T, Stuckler F, Ried J, Adamski J, et al. Gender-specific pathway differences in the human serum metabolome. Metabolomics: Official journal of the Metabolomic Society. 2015; 11:1815-33.
[24] Mittelstrass K, Ried J S, Yu Z, Krumsiek J, Gieger C, Prehn C, et al. Discovery of Sexual Dimorphisms in Metabolic and Genetic Biomarkers. PLOS Genetics. 2011; 7:e1002215.
[25] Johnson L A, Torres E R, Impey S, Stevens J F, Raber J. Apolipoprotein E4 and Insulin Resistance Interact to Impair Cognition and Alter the Epigenome and Metabolome. Scientific reports. 2017; 7:43701.
[26] Kochhar S, Jacobs D M, Ramadan Z, Berruex F, Fuerholz A, Fay L B. Probing gender-specific metabolism differences in humans by nuclear magnetic resonance-based metabonomics. Analytical biochemistry. 2006:352:274-81.
[27] Dunn W B, Lin W, Broadhurst D, Begley P, Brown M, Zelena E, et al. Molecular phenotyping of a UK population: defining the human serum metabolome. Metabolomics: Official journal of the Metabolomic Society. 2015; 11:9-26.
[28] Gonzalez-Covarrubias V, Beekman M, Uh H-W, Dane A, Troost J, Paliukhovich I, et al. Lipidomics of familial longevity. Aging Cell. 2013; 12:426-34.
[29] Menni C, Kastenmuller G, Petersen A K, Bell J T, Psatha M, Tsai P C, et al. Metabolomic markers reveal novel pathways of ageing and early development in human populations. Intemational journal of epidemiology. 2013; 42:1111-9.
[30] Cai H, Cong W N, Ji S, Rothman S, Maudsley S, Martin B. Metabolic dysfunction in Alzheimer's disease and related neurodegenerative disorders. Current Alzheimer research. 2012; 9:5-17.
[31] Sonntag K-C, Ryu W-I, Amirault K M, Healy R A, Siegel A J, McPhie D L, et al. Late-onset Alzheimer's disease is associated with inherent changes in bioenergetics profiles. Scientific reports. 2017; 7:14038.
[32] Procaccini C, Santopaolo M, Faicchia D, Colamatteo A, Formisano L, de Candia P, et al. Role of metabolism in neurodegenerative disorders. Metabolism: clinical and experimental. 2016; 65:1376-90.
[33] Yu Z, Zhai G, Singmann P, He Y, Xu T, Prehn C, et al. Human serum metabolic profiles are age dependent. Aging Cell. 2012; 11:960-7.
[34] Sekula P, Goek O N, Quaye L, Barrios C, Levey A S, Romisch-Margl W, et al. A Metabolome-Wide Association Study of Kidney Function and Disease in the General Population. J Am Soc Nephrol. 2016; 27:1175-88.
[35] Tudorache I F, Trusca V G, Gafencu A V. Apolipoprotein E-A Multifunctional Protein with Implications in Various Pathologies as a Result of Its Structural Features. Computational and structural biotechnology journal. 2017; 15:359-65.
[36] Teslovich T M, Musunuru K, Smith A V, Edmondson A C, Stylianou I M, Koseki M, et al. Biological, clinical and population relevance of 95 loci for blood lipids. Nature. 2010; 466:707-13.
[37] Shin S Y, Fauman E B, Petersen A K, Krumsiek J, Santos R, Huang J, et al. An atlas of genetic influences on human blood metabolites. Nature genetics. 2014; 46:543-50.
[38] Mielke M M, Haughey N J, Han D, An Y, Bandaru W R, Lyketsos C G, et al. The Association Between Plasma Ceramides and Sphingomyelins and Risk of Alzheimer's Disease Differs by Sex and APOE in the Baltimore Longitudinal Study of Aging. Journal of Alzheimer's disease: JAD. 2017; 60:819-28.
[39] Long T, Hicks M, Yu H C, Biggs W H, Kirkness E F, Menni C, et al. Whole-genome sequencing identifies common-to-rare variants associated with human blood metabolites. Nature genetics. 2017; 49:568-78.
[40] Mielke M M, Bandaru W, Haughey N J, Xia J, Fried L P, Yasar S, et al. Serum ceramides increase the risk of Alzheimer disease: the Women's Health and Aging Study I I. Neurology. 2012; 79:633-41.
[41] Tynkkynen J, Chouraki V, van der Lee S J, Hemesniemi J, Yang Q, Li S, et al. Association of branched-chain amino acids and other circulating metabolites with risk of incident dementia and Alzheimer's disease: A prospective study in eight cohorts. Alzheimer's & dementia: the journal of the Alzheimer's Association. 2018; 14:723-33.
[42] Trushina E, Dutta T, Persson X-M T, Mielke M M, Petersen R C. Identification of Altered Metabolic Pathways in Plasma and CSF in Mild Cognitive Impairment and Alzheimer's Disease Using Metabolomics. PLOS ONE. 2013; 8:e63644.
[43] Oresic M, Hyotylainen T, Herukka S K, Sysi-Aho M, Mattila I, Seppanan-Laakso T, et al. Metabolome in progression to Alzheimer's disease. Translational psychiatry. 2011; 1:e57.
[44] Toledo J B, Arnold M, Kastenmuller G, Chang R, Baillie R A, Han X, et al. Metabolic network failures in Alzheimer's disease: A biochemical road map. Alzheimer's & dementia: the journal of the Alzheimer's Association. 2017; 13:965-84.
[45] Gonzclez-Dominguez R, Garcia-Barrera T, Gómez-Ariza J L. Combination of metabolomic and phospholipid-profiling approaches for the study of Alzheimer's disease. Journal of Proteomics. 2014; 104:37-47.
[46] Whiley L, Sen A, Heaton J, Proitsi P, Garcia-Gómez D, Leung R, et al. Evidence of altered phosphatidylcholine metabolism in Alzheimer's disease. Neurobiology of Aging. 2014; 35:271-8.
[47] Proitsi P, Kim M, Whiley L, Pritchard M, Leung R, Soininen H, et al. Plasma lipidomics analysis finds long chain cholesteryl esters to be associated with Alzheimer's disease. Translational psychiatry. 2015; 5:e494.
[48] Graham S F, Chevallier O P, Elliott C T, Hölscher C, Johnston J, McGuinness B, et al. Untargeted Metabolomic Analysis of Human Plasma Indicates Differentially Affected Polyamine and L-Arginine Metabolism in Mild Cognitive Impairment Subjects Converting to Alzheimer's Disease. PLOS ONE. 2015; 10:e0119452.
[49] Varma V R, Oommen A M, Varma S, Casanova R, An Y, Andrews R M, et al. Brain and blood metabolite signatures of pathology and progression in Alzheimer disease: A targeted metabolomics study. PLoS Med. 2018; 15:e1002482.
[50] Pan X, Elliott C T, McGuinness B, Passmore P, Kehoe P G, Hölscher C, et al. Metabolomic Profiling of Bile Acids in Clinical and Experimental Samples of Alzheimer's Disease. Metabolites. 2017; 7.
[51] MahmoudianDehkordi S, Arnold M, Nho K, Ahmad S, Jia W, Xie G, et al. Altered bile acid profile associates with cognitive impairment in Alzheimer's disease—An emerging role for gut microbiome. Alzheimer's & dementia: the journal of the Alzheimer's Association. 2018.
[52] Nho K, Kueider-Paisley A, MahmoudianDehkordi S, Arnold M, Risacher S L, Louie G, et al. Altered bile acid profile in mild cognitive impairment and Alzheimer's disease: Relationship to neuroimaging and CSF biomarkers. Alzheimers & dementia: the journal of the Alzheimers Association. 2018.
[53] Jack C R, Jr., Bennett D A, Blennow K, Carrillo M C, Dunn B, Budd Haeberlein S, et al. NIA-A A research framework: Towards a biological definition of Alzheimer's disease. Alzheimer's Association International Conference. London, England: Alzheimers Association; 2017. p. 1-57.
[54] Jack C R, Jr., Bennett D A, Blennow K, Carrillo M C, Feldman H H, Frisoni G B, et al. A/T/N: An unbiased descriptive classification scheme for Alzheimer disease biomarkers. Neurology. 2016; 87:539-47.
[55] Weiner M W, Veitch D P, Aisen P S, Beckett L A, Cairns N J, Cedarbaum J, et al. Impact of the Alzheimer's Disease Neuroimaging Initiative, 2004 to 2014. Alzheimer's & dementia: the journal of the Alzheimer's Association. 2015; 11:865-84.
[56] St John-Williams L, Blach C, Toledo J B, Rotroff D M, Kim S, Klavins K, et al. Targeted metabolomics and medication classification data from participants in the ADNI1 cohort. Sci Data. 2017; 4:170140.
[57] Hansson O, Seibyl J, Stomrud E, Zetterberg H, Trojanowski J Q, Bittner T, et al. CSF biomarkers of Alzheimer's disease concord with amyloid-beta PET and predict clinical progression: A study of fully automated immunoassays in BioFINDER and ADNI cohorts. Alzheimer's & dementia: the journal of the Alzheimer's Association. 2018; 14:1470-81.
[58] Bittner T, Zetterberg H, Teunissen C E, Ostlund R E, Jr., Militello M, Andreasson U, et al. Technical performance of a novel, fully automated electrochemiluminescence immunoassay for the quantitation of beta-amyloid (1-42) in human cerebrospinal fluid. Alzheimer's & dementia: the journal of the Alzheimer's Association. 2016; 12:517-26.
[59] Landau S M, Harvey D, Madison C M, Koeppe R A, Reiman E M, Foster N L, et al. Associations between cognitive, functional, and FDG-PET measures of decline in A D and MCI. Neurobiol Aging. 2011; 32:1207-18.
[60] Jagust W J, Bandy D, Chen K, Foster N L, Landau S M, Mathis C A, et al. The Alzheimer's Disease Neuroimaging Initiative positron emission tomography core. Alzheimer's & dementia: the journal of the Alzheimer's Association. 2010; 6:221-9.
[61] Li J, Ji L. Adjusting multiple testing in multilocus analyses using the eigenvalues of a correlation matrix. Heredity (Edinb). 2005; 95:221-7.
[62] Paternoster R, Brame R, Mazerolle P, Piquero A. Using the Correct Statistical Test for the Equality of Regression Coefficients. Criminology. 1998; 36:859-66.
[63] Higgins J P, Thompson S G. Quantifying heterogeneity in a meta-analysis. Stat Med. 2002; 21:1539-58.
[64] Sharma S, Black S M. Camitine Homeostasis, Mitochondrial Function, and Cardiovascular Disease. Drug Discov Today Dis Mech. 2009; 6:e31-e9.
[65] Lynch C J, Adams S H. Branched-chain amino acids in metabolic signalling and insulin resistance. Nat Rev Endocrinol. 2014; 10:723-36.
[66] Adeva-Andany M, Souto-Adeva G, Ameneiros-Rodriguez E, Fernandez-Fernandez C, Donapetry-Garcia C, Dominguez-Montero A. Insulin resistance and glycine metabolism in humans. Amino Acids. 2018:50:11-27.
[67] Phang J M, Liu W, Zabimyk O. Proline metabolism and microenvironmental stress. Annu Rev Nutr. 2010; 30:441-63.
[68] Feng R N, Niu Y C, Sun X W, Li Q, Zhao C, Wang C, et al. Histidine supplementation improves insulin resistance through suppressed inflammation in obese women with the metabolic syndrome: a randomised controlled trial. Diabetologia. 2013; 56:985-94.
[69] Watanabe M, Suliman M E, Qureshi A R, Garcia-Lopez E, Barany P, Heimburger O, et al. Consequences of low plasma histidine in chronic kidney disease patients: associations with inflammation, oxidative stress, and mortality. Am J Clin Nutr. 2008; 87:1860-6.
[70] Huang H, Vandekeere S, Kalucka J, Bierhansl L, Zecchin A, Bruning U, et al. Role of glutamine and interlinked asparagine metabolism in vessel formation. EMBO J. 2017:36:2334-52.
[71] Ruth M R, Field C J. The immune modifying effects of amino acids on gut-associated lymphoid tissue. J Anim Sci Biotechnol. 2013; 4:27.
[72] Newgard C B, An J, Bain J R, Muehlbauer M J, Stevens R D, Lien L F, et al. A branched-chain amino acid-related metabolic signature that differentiates obese and lean humans and contributes to insulin resistance. Cell Metab. 2009; 9:311-26.
[73] Lacruz M E, Kluttig A, Tiller D, Medenwald D, Giegling I, Rujescu D, et al. Instability of personal human metabotype is linked to all-cause mortality. Scientific reports. 2018; 8:9810.
[74] Mapstone M, Cheema A K, Fiandaca M S, Zhong X, Mhyre T R, MacArthur L H, et al. Plasma phospholipids identify antecedent memory impairment in older adults. Nat Med. 2014:20:415-8.
[75] Casanova R, Varma S, Simpson B, Kim M, An Y, Saldana S, et al. Blood metabolite markers of preclinical Alzheimer's disease in two longitudinally followed cohorts of older individuals. Alzheimer's & dementia: the journal of the Alzheimer's Association. 2016; 12:815-22.
[76] Tapiola T, Alafuzoff I, Herukka S K, Parkkinen L, Hartikainen P, Soininen H, et al. Cerebrospinal fluid {beta}-amyloid 42 and tau proteins as biomarkers of Alzheimer-type pathologic changes in the brain. Arch Neurol. 2009; 66:382-9.

### EXAMPLES

### Example 1

### Study Participants

Data used in the preparation of this article were obtained from the ADNI database. In the current study, we included 1,517 baseline serum samples of fasting participants pooled from ADNI phases 1, GO, and 2. Demographics, diagnostic groups, and numbers and distributions of key risk factors are provided in Table 1. AD dementia diagnosis was established based on the NINDS-ADRDA criteria for probable AD. Mild cognitive impairment (MCI) participants did not meet these AD criteria and had largely intact functional performance, meeting predetermined criteria for amnestic MCI [55]. Of the 1,517 subjects, 689 were female and 828 were male, with 708 APOE ε4 carriers and 809 non-carriers. In the combined stratification by sex and APOE ε4 status (APOE4⁻=0 copies of ε4, APOE4⁺=1 or 2 copies of ε4), the APOE ε4 non-carriers were separated into 374 females and 435 males, while of APOE ε4 carriers 315 were female and 393 were male.

TABLE 1
Characteristics of the 1,517 ADNI samples included in this study

| | Global | | | | | |
|---|---|---|---|---|---|---|
| | Dataset | CN | SMC | EMCI | MCI | AD |
| N_{subjects} | 1517 | 362 | 93 | 270 | 490 | 302 |
| Sex (m/f) | 828/689 | 177/185 | 39/54 | 149/121 | 298/192 | 165/137 |
| Age | 73.72 | 74.61 | 72.34 | 71.26 | 74.03 | 74.79 |
| | (± 7.25) | (± 5.77) | (± 5.70) | (± 7.63) | (± 7.63) | (± 7.77) |
| BMI | 26.86 | 26.99 | 28.46 | 27.96 | 26.45 | 25.88 |
| | (± 4.82) | (± 4.53) | (± 6.23) | (± 5.36) | (± 4.27) | (± 4.69) |
| Education | 15.88 | 16.24 | 16.78 | 15.95 | 15.84 | 15.16 |
| | (± 2.87) | (± 2.79) | (±2.55) | (± 2.67) | (± 2.91) | (± 3.01) |
| APOE4−/+ | 809/708* | 261/101 | 64/29 | 155/115 | 224/266 | 105/197 |
| CSF | 1082* | 236 | 84 | 245 | 308 | 209 |
| available | | | | | | |
| Path.Abeta− | 407/675 | 134/102 | 57/27 | 122/123 | 75/233 | 19/190 |
| /+ | | | | | | |
| CSF Abeta | 1052.73 | 1324.60 | 1395.01 | 1172.73 | 896.35 | 697.95 |
| | (± 601.70) | (± 652.13) | (± 618.19) | (± 569.12) | (± 501.80) | (± 431.49) |
| CSF p-Tau | 27.79 | 22.01 | 21.66 | 24.34 | 30.81 | 36.38 |
| | (±14.56) | (± 9.19) | (± 9.14) | (± 14.03) | (± 14.94) | (± 16.07) |
| FDG-PET | 1143* | 247 | 93 | 268 | 318 | 217 |
| available | | | | | | |
| FDG-PET | 6.17 (± 0.77) | 6.53 (± 0.58) | 6.60 (± 0.58) | 6.44 (± 0.60) | 6.08 (± 0.68) | 5.36 (± 0.73) |

*Numbers for combined stratification;

| | APOE4− | APOE4− | APOE4+ | APOE4+ |
|---|---|---|---|---|
| | females | males | females | males |
| Total | 374 | 435 | 315 | 393 |
| CSF | 267 | 315 | 222 | 278 |
| available | | | | |
| FDG-PET | 278 | 337 | 230 | 298 |
| available | | | | |

CN: cognitively normal; SMC: subjective memory complaints; EMU: early mild cognitive impairment;
MCI: mild cognitive impairment; AD: probably Alzheimer’s disease; BMI: body-mass-index; APOE4 ±:
non-carriers and carriers of the APOE ε4 allele. respectively.

### Metabolomics Data Acquisition

Metabolites were measured with the targeted AbsoluteIDQ-p180 metabolomics kit (BIOCRATES Life Science AG, Innsbruck, Austria), with an ultra-performance liquid chromatography (UPLC)/MS/MS system (Acquity UPLC (Waters), TQ-S triple quadrupole MS/MS (Waters)) which provides measurements of up to 186 endogenous metabolites. Sample extraction, metabolite measurement, identification, quantification, and primary quality control (QC) followed standard procedures as described before [44, 56].

### Metabolomics Data Processing

Metabolomics data processing followed the processing protocol previously described [44, 56] with a few adjustments. In brief, raw metabolomics data for 182 metabolites was available for 1,681 serum study samples and, for each plate, 2-3 NIST Standard Reference samples were available. Furthermore, we had blinded duplicated measurements for 19 samples (ADNI-1) and blinded triplicated measurements for 17 samples (ADNI-GO and -2) distributed across plates. We first excluded 22 metabolites with large numbers of missing values (>40%). Then, we removed plate batch effects using cross-plate mean normalization using NIST metabolite concentrations. Duplicated and triplicated study samples were then used to calculate the coefficients of variation (exclusion criterion >20%) and intra-class correlation (exclusion criterion <0.65) for each metabolite. We removed 20 metabolites that violated these thresholds. Next, we excluded non-fasting samples (n=108), imputed missing metabolite data using half the value of the lower limit of detection per metabolite and plate, log 2-transformed metabolite concentrations, centered and scaled distributions to a mean of zero and unit variance and winsorized single outlying values to 3 standard deviations. We then used the Mahalanobis distance for detection of multivariate subject outliers, applying the critical Chi-square value for p<0.01 and removing 42 subjects. Finally, metabolites were adjusted for significant medication effects using stepwise backwards selection (see [56]). The final QC-ed metabolomics dataset was further restricted to individuals having data on all significant covariates, resulting in the study dataset of 140 metabolites and 1,517 individuals.

### Phenotype Data and Covariate Selection

We limited association analyses of metabolites with AD to early detectable endophenotypes, more specifically to the pathological threshold for CSF Aβ₁₋₄₂, levels of phosphorylated tau protein in the CSF (p-tau), and brain glucose metabolism measured by [18F]fluorodeoxyglucose (FDG)-positron emission tomography (PET). Baseline data on these biomarkers for ADNI-1, -GO, and -2 participants was downloaded from the LONI online portal (ida.loni.usc.edu). For CSF biomarker data, we used the dataset generated using the validated and highly automated Roche Elecsys electrochemiluminescence immunoassays [57, 58]. For FDG-PET, we used a ROI-based measure of average glucose uptake across the left and right angular, left and right temporal and bilateral posterior cingulate regions derived from preprocessed scans (co-registered, averaged, standardized image and voxel size, uniform resolution) and intensity-normalized using a pons ROI to obtain standard uptake value ratio (SUVR) means [59, 60]. The pathological CSF Aβ₁₋₄₂ cut-point (1,073 pg/mL) as reported by the ADNI biomarker core for diagnosis-independent mixture modeling (see adni.loni.usc.edu/methods/) was used for categorization since CSF Aβ₁₋₄₂ concentrations were not normally distributed. Processed FDG-PET values were scaled and centered to zero mean and unit variance prior to association analysis, p-tau levels were additionally log 2-transformed. Furthermore, we extracted covariates including age, sex, body-mass-index (BMI; calculated using baseline weight and body height), number of copies of the APOE e4 genotype, and years of education. Covariates were separated into forced-in (age, sex, ADNI study phase, and number of copies of APOE e4) and covariates (BMI, education) selectable by backwards selection. ADNI study phase was included to adjust for remaining metabolic differences between batches (ADNI-1 and ADNI-GO/-2 were processed in separate runs), as well as differences in PET imaging technologies.

### Association Analyses

Association analyses of the three AD biomarkers with metabolite levels were conducted using standard linear (p-tau, FDG-PET) and logistic (pathological Aβ₁₋₄₂) regression. For pathological CSF Aβ₁₋₄₂, only BMI was additionally selected, while for p-tau and FDG-PET the full set of covariates was used. The stratification variables sex and copies of APOE ε4 were excluded as covariates in the respective group-specific association analyses (i.e., sex in sex-stratified and copies of APOE ε4 in APOE4± status-stratified analyses, respectively). For identifying metabolic sex-differences, we used linear regression with metabolite levels as the dependent variable and age, sex, BMI, ADNI study phase, and diagnostic group as explanatory variables and retrieved statistics for sex. To adjust for multiple testing, we accounted for the significant correlation structure across the 140 metabolites and determined the number of independent metabolic features (i.e., tests) using the method of Li and Ji [61] to be 55, leading to a threshold of Bonferroni significance of 9.09×10⁻⁴. To assess significance of heterogeneity between strata, we followed the methodology of [24, 62] that is similar to the determination of study heterogeneity in inverse-weighted meta-analysis. We further provide a scaled index of percent heterogeneity that is similar to the I² statistic [63].

### Bootstrapping Analysis

Bootstrapping was performed using ordinary nonparametric bootstraps for each of the three A-T-N biomarkers separately. For this, we drew random indices with replacement a 1000 times from all participants in ADNI with the biomarker available. Association analysis was performed on each bootstrap using the same regression models as described above. We then calculated the bias of the effect estimates (i.e., the difference between effect estimates obtained in the original analyses and the respective average effect estimate across all bootstraps), as well as the bootstrap-t (or studentized) 95% confidence interval that is taking into account the variance of the estimates in each single bootstrap. Averaged bootstrap statistics were obtained using the mean of the beta estimates and the mean of their standard errors across the set of 1000 bootstraps and using their ratio as statistic to retrieve associated two-tailed p values from the standard normal distribution.

### Power Analysis

In each power analysis, we transformed covariate-adjusted effect sizes to sample size-weighted standardized effects (Cohen's d). For metabolic sex differences, we calculated the power for two-sample t tests to identify significant sexual dimorphisms for metabolites with the standardized effect sizes observed in the pooled ADNI samples at Bonferroni significance in CN participants, participants with MCI, and patients with probable AD. To obtain estimates of sample sizes required to replicate metabolite associations and heterogeneity estimates, we used the same approach with power fixed to 50% (the post hoc/observed power to find results at p values equal or below the respective applied threshold, i.e., nominal or Bonferroni significance). Thereby, we estimated sample sizes assuming perfectly balanced data sets (with respect to sex and APOE ε4 status). This is a very rough approximation as it further assumes that the effect sizes reported for ADNI are generalizable to any replication cohort. Therefore, reported required sample sizes may deviate in reality.

### Replication Analysis in ROS/MAP

Replication analysis in ROS/MAP and AIBL. The ROS/MAP studies are both longitudinal cohort studies of aging and AD at Rush University and are designed to be used in joint analyses to maximize sample size. Both studies were approved by an Institutional Review Board of Rush University Medical Center. All participants signed an informed consent and a repository consent to allow their biospecimens and data to be used for ancillary studies. We measured metabolite levels using the AbsoluteIDQ-p180 metabolomics kit in 596 serum samples from 559 participants (37 additional samples from follow-up visits). Brain amyloid pathology data were available for 89 participants (126 serum samples) comprised of 40 CN, 28 MCI, and 21 AD participants; 100 participants (137 serum samples) had brain tau pathology data (46 CN, 28 MCI, and 26 AD). To obtain maximal power for replication, we included longitudinal metabolomics data where available and applied linear mixed models for association analysis. We used the same covariates as in ADNI, including study phase (ROS or MAP), sex, age at visit, BMI, copies of APOE ε4, and education (only for tau pathology). Race was added as additional covariate. Random effects (intercept) in the mixed models were included for both visit and participant identifiers.

AIBL is a longitudinal study of over 1100 people assessed over >4.5 years to determine which biomarkers, cognitive characteristics, and health and lifestyle factors determine subsequent development of symptomatic AD. The AIBL study was approved by the institutional ethics committees of Austin Health, St. Vincent's Health, Hollywood Private Hospital and Edith Cowan University, and all volunteers gave written informed consent before participating in the study. We had access to measurements of CSF p-tau for 94 participants (82 CN, 7 MCI, and 5 AD) with lipidomic data available. In contrast to ADNI, lipidomic data in AIBL was assessed on the UHPLC-MS/MS platform of the Metabolomics Laboratory of the Baker Heart and Diabetes Institute, Melbourne, Australia, and not the AbsoluteIDQ-p180 metabolomics kit. As a consequence, matching measures of only three metabolites (PC ae C36:1, PC ae C36:2, and SM (OH) C16:1) could be derived in AIBL and were available for replication. Association analysis was performed for log-transformed CSF p-tau levels and the three metabolite measures using linear regression while adjusting for sex, age, BMI, APOE ε4 status, and education. For both ROS/MAP and AIBL, sex and APOE e4 respectively, were omitted as covariates in stratified analyses and heterogeneity estimates were calculated as in ADNI.

TABLE 2
Replication Analysis in ROS/MAP - Results of association analysis and heterogeneity estimation

| pathological | | pooled analysis | | | males | | | females | | |
|---|---|---|---|---|---|---|---|---|---|---|
| marker | metabolite | effect | se | p-value | effect | se | p-value | effect | se | p-value |
| Overall | PC ae C44:4 | 0.031 | 0.009 | 1.23E−03 | 0.058 | 0.019 | 6.84E−03 | 0.022 | 0.011 | 0.061 |
| amyloid level | PC ae C44:5 | 0.043 | 0.013 | 1.42E−03 | 0.086 | 0.025 | 3.16E−03 | 0.027 | 0.016 | 0.107 |
| | PC ae C44:6 | 0.049 | 0.012 | 8.63E−05 | 0.085 | 0.023 | 2.16E−03 | 0.037 | 0.015 | 0.018 |
| | PC ae C42:4 | 0.040 | 0.009 | 3.31E−05 | 0.067 | 0.019 | 2.79E−03 | 0.025 | 0.011 | 0.030 |
| | Threonine | −0.003 | 0.012 | 0.781 | 0.012 | 0.018 | 0.528 | −0.003 | 0.015 | 0.836 |
| | Valine | −0.018 | 0.009 | 0.049 | 0.001 | 0.015 | 0.962 | −0.023 | 0.012 | 0.070 |
| | Glycine | −0.010 | 0.012 | 0.427 | | | | | | |
| | Proline | −0.024 | 0.014 | 0.100 | | | | | | |
| Severity of | C2 | −0.047 | 0.039 | 0.231 | −0.041 | 0.059 | 0.497 | −0.051 | 0.055 | 0.355 |
| tau pathology | C5-DC (C6- | −0.022 | 0.028 | 0.436 | 0.002 | 0.045 | 0.969 | −0.052 | 0.034 | 0.131 |
| | OH) | | | | | | | | | |
| | C8 | −0.037 | 0.057 | 0.519 | 0.098 | 0.106 | 0.368 | −0.113 | 0.072 | 0.125 |
| | C10 | −0.056 | 0.071 | 0.432 | 0.133 | 0.137 | 0.345 | −0.154 | 0.089 | 0.091 |
| | PC ae C36:2 | 0.047 | 0.025 | 0.060 | −0.013 | 0.038 | 0.744 | 0.080 | 0.033 | 0.018 |
| | PC ae C36:1 | 0.040 | 0.018 | 0.030 | 0.023 | 0.027 | 0.414 | 0.046 | 0.024 | 0.064 |
| | SM (OH) | 0.047 | 0.020 | 0.021 | 0.034 | 0.030 | 0.271 | 0.064 | 0.026 | 0.017 |
| | C16:1 | | | | | | | | | |
| | Glycine | −0.025 | 0.037 | 0.505 | 0.047 | 0.037 | 0.225 | −0.070 | 0.052 | 0.186 |
| | Histidine | −0.019 | 0.026 | 0.475 | −0.047 | 0.042 | 0.275 | 0.007 | 0.034 | 0.825 |
| | Asparagine | −0.062 | 0.031 | 0.048 | −0.095 | 0.041 | 0.031 | −0.029 | 0.044 | 0.501 |
| sex difference | | | interaction | APOE ε4+ | | | | | | |
| t | p-value | I² | p-value | effect | se | p-value | | | | |
| 1.645 | 0.100 | 39.223 | 0.225 | 0.007 | 0.022 | 0.746 | | | | |
| 1.987 | 0.047 | 49.672 | 0.057 | 0.010 | 0.035 | 0.776 | | | | |
| 1.712 | 0.087 | 41.604 | 0.166 | 0.023 | 0.034 | 0.512 | | | | |
| 1.903 | 0.057 | 47.439 | 0.043 | 0.005 | 0.022 | 0.819 | | | | |
| 0.630 | 0.529 | 0.000 | 0.753 | | | | | | | |
| 1.221 | 0.222 | 18.074 | 0.510 | | | | | | | |
| | | | | −0.011 | 0.024 | 0.646 | | | | |
| | | | | 0.007 | 0.029 | 0.816 | | | | |
| 0.128 | 0.898 | 0.000 | 0.955 | | | | | | | |
| 0.960 | 0.337 | 0.000 | 0.110 | | | | | | | |
| 1.647 | 0.100 | 39.267 | 0.111 | | | | | | | |
| 1.754 | 0.079 | 42.985 | 0.159 | | | | | | | |
| −1.842 | 0.065 | 45.716 | 0.078 | | | | | | | |
| −0.639 | 0.523 | 0.000 | 0.909 | | | | | | | |
| −0.758 | 0.448 | 0.000 | 0.789 | | | | | | | |
| 1.820 | 0.069 | 45.066 | 0.170 | | | | | | | |
| −1.015 | 0.310 | 1.445 | 0.101 | | | | | | | |
| −1.091 | 0.275 | 8.357 | 0.177 | | | | | | | |
| | | | APOE ε4 status | | | | | | | |
| APOE ε4- | | | difference | | | interaction | | | | |
| effect | se | p-value | t | p-value | I² | p-value | | | | |
| 0.035 | 0.009 | 2.10E−04 | 1.139 | 0.255 | 12.237 | 0.215 | | | | |
| 0.044 | 0.012 | 6.00E−04 | 0.933 | 0.351 | 0.000 | 0.298 | | | | |
| 0.050 | 0.011 | 3.87E−05 | 0.747 | 0.455 | 0.000 | 0.375 | | | | |
| 0.044 | 0.009 | 1.49E−05 | 1.645 | 0.100 | 39.205 | 0.063 | | | | |
| −0.009 | 0.013 | 0.504 | 0.105 | 0.916 | 0.000 | 0.738 | | | | |
| −0.028 | 0.015 | 0.066 | −1.068 | 0.286 | 6.332 | 0.231 | | | | |

We then performed a targeted analysis to replicate associations of PC ae C44:4, PC ae C44:5, and PC ae C44:6 with Aβ₁₋₄₂ pathology using post-mortem, neuropathology-derived measures of total amyloid load in the brain. This phenotype was transformed to square root values to get values closer to a normal distribution. Linear regression models were adjusted for age at blood draw, sex, study cohort (ROS vs. MAP), race, number of copies of APOE ε4, as well as years of education. All three p-values were Bonferroni significant when adjusting for three test (p-value threshold of p<1.667), complete result statistics were:

| biomarker | metabolite | effect | se | p-value |
|---|---|---|---|---|
| total amyloid | PC ae C44:4 | 0.30741 | 0.10277 | 0.00373 |
| in the brain | PC ae C44:5 | 0.2656 | 0.10257 | 0.01149 |
| | PC ae C44:6 | 0.30992 | 0.10212 | 0.00328 |

### Replication Analysis in ABL

We had access to measurements of CSF p-tau for 94 subjects (82 CN, 7 MCI, and 5 AD) in conjunction with targeted, quantitative lipidomics data (UHPLC ESI-MS/MS platform of the Metabolomics Laboratory of the Baker Heart and Diabetes Institute, Melbourne, Australia). The applied lipidomics technology provides greater resolution than the p180 (which reports many lipids in form of sums of fatty acid chains). With the available data, we were able to derive measures of three metabolites (PC ae C36:1, PC ae C36:2, and SM (OH) C16:1) by summing up the, partly fractionized, levels of the following lipids:

### PC ae C36:1

100%—PC(O-18:0/18:1)
100%—PC(15-MHDA_18:1)
100%—PC(17:0_18:1)
44%—SM(d16:1/23:0)/SM(d17:1/22:0)

### PC ae C36:2

100%—PC(O-18:1/18:1)
100%—PC(O-18:0/18:2)
100%—PC(15-MHDA_18:2)
100%—PC(17:0_18:2)

### SM (OH) C16:1

100%—SM(d16:1/19:0)
100%—SM(d18:1/17:0)+SM(d17:1/18:0)

To ascertain that the thus retrieved sums/metabolite measures are comparable, we performed regression analysis (Table 3) of the derived measures against the corresponding p180 metabolites in ADNI-1 for which data on both platforms are available. Overall, R²-values for these comparisons were >60%, corresponding to an estimated overall correlation of >77.45%, which provides strong evidence for the applicability of this approach.

Of the 94 total subjects available for CSF p-tau analysis, 48 were female and 46 male and 72 APOE ε4- and 22 APOE ε4+; mean age was 73.9 (±5.8) years. CSF p-tau data was obtained by analyzing CSF samples in duplicate using the enzyme-linked immunosorbent assay (ELISA): INNOTEST PHOSPHO-TAU(181P) (P-tau181P) (Innogenetics, Ghent, Belgium).

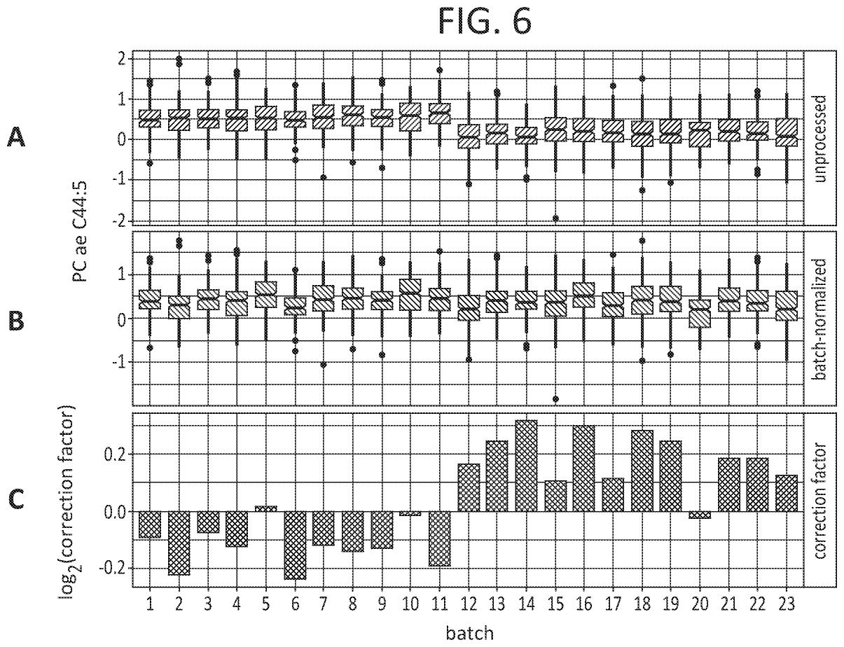

Metabolomics data processing was performed very similar as for the ADNI, except that we used pooled plasma quality control (QC) sample-based median quotient normalization for batch removal instead of utilizing NIST standard plasma (exemplified in FIG. 6A-C). We then did a targeted analysis to replicate associations of the three metabolite measures with CSF p-tau. Association analysis was performed for log-transformed CSF p-tau levels and the three metabolite measures using linear regression while adjusting for sex, age, BMI, APOE ε4 status, and level of education (categorized in 5 groups: 0=0 to 6 years of education, 1=7 to 8 years, 2=9 to 12 years, 3=13 to 15 years and 4=15 years+). In sex-stratified analyses, sex was omitted as covariate. Heterogeneity estimates were calculated as in ADNI.

TABLE 3
Replication Analysis in AUK-Results of association analysis and
heterogeneity estimation

| | | pooled analysis | | | males | | |
|---|---|---|---|---|---|---|---|
| bio- | metab- | | | p- | | | p- |
| marker | olite | effect | se | value | effect | se | value |
| CSF | PC ae | −0.014 | 0.110 | 0.903 | 0.133 | 0.145 | 0.363 |
| p-tau | C36:1 | | | | | | |
| | PC ae | −0.124 | 0.110 | 0.261 | 0.061 | 0.154 | 0.692 |
| | C36:2 | | | | | | |
| | SM (OH) | −0.039 | 0.109 | 0.724 | 0.191 | 0.150 | 0.210 |
| | C16:1 | | | | | | |
| females | | | sex difference | | | interaction | |
| effect | se | p-value | t | p-value | I² | p-value | |
| −0.240 | 0.152 | 0.122 | 1.779 | 0.075 | 43.783 | 0.087 | |
| −0.296 | 0.151 | 0.057 | 1.659 | 0.097 | 39.705 | 0.111 | |
| −0.274 | 0.150 | 0.076 | 2.190 | 0.029 | 54.328 | 0.016 | |

### Example 2

In this study, we used CSF biomarkers, FDG-PET imaging, and metabolomics data on 140 metabolites to investigate metabolic effects in relation to sex and AD and their interaction. Out of 1,517 ADNI participants, 1,082 had CSF Aβ₁₋₄₂ and p-tau levels and 1,143 had FDG-PET data available (Table 1, supra). We included all individuals with respective data regardless of their diagnostic classification, as we were interested in these three representatives of the A-T-N AD biomarker schema [53, 54] as our main readouts. In this data set, there was no significant difference in the number of APOE4±subjects between females and males. Of the three AD biomarkers, only p-tau levels were significantly different between sexes (corrected P=0.01) with slightly higher levels observed in females.

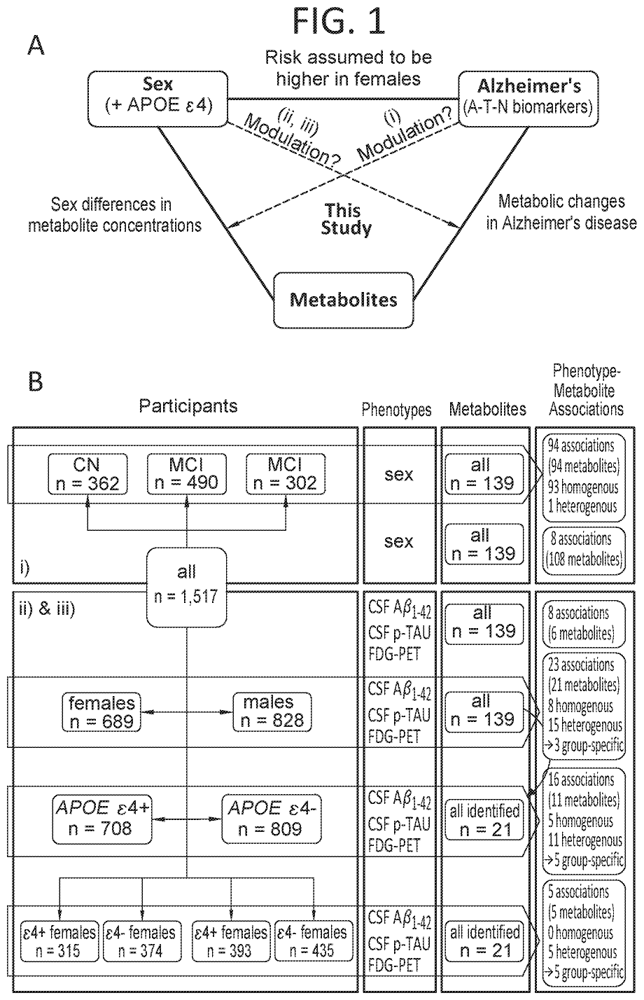

Previous studies consistently showed widespread metabolic sex-differences, metabolic imprint of genetic variance in the APOE locus, as well as significant associations between blood metabolites and AD biomarkers that are independent of (i.e., adjusted for) sex. In the current study, we add the specific examination of the following central questions (FIG. 1A): (i) Are metabolic sex-differences changed due to presence of (probable) AD?, (ii) Are metabolite associations with A-T-N biomarkers modified by sex?, and (iii) Is there evidence for APOE4 status influencing metabolite associations with A-T-N biomarkers that show differences between sexes?

To address the three research questions of this study (FIG. 1B), we first performed analyses of sex-metabolite associations for 139 metabolites in the ADNI cohort stratified by diagnostic group (question i). Subsequently, we performed phenotype (A-T-N)-metabolite associations for 139 metabolites in the ADNI cohort stratified by sex (question ii) and stratified by APOE ε4 status; additionally, we performed phenotype (A-T-N)-metabolite associations for the 21 significantly associated metabolites after stratification by sex plus APOE ε4 status (question iii).

### No Significant Change of Metabolic Sex Differences in AD

In a first step, we tested whether sex-associated differences in blood metabolite levels differ between patients with probable AD, subjects with late MCI, and CN subjects in the ADNI cohorts. In the complete cohort (n=1,517), we found 108 of 139 metabolites to be significantly associated with sex after multiple testing correction while adjusting for age, BMI, ADNI study phase, and diagnostic group. 70 of these associations replicate previous findings in a healthy population using a prior version of the same metabolomics platform [24] that provides measurements on 92 out of the 108 metabolites identified in ADNI. All SMs and the majority of PCs were more abundant in women. The majority of biogenic amines, amino acids and acylcarnitines were more abundant in men.

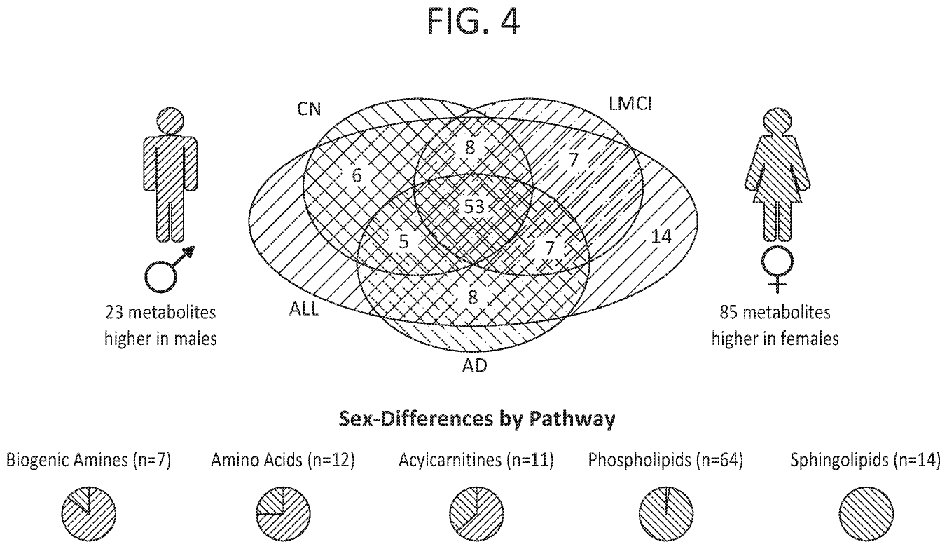

Stratifying subjects by diagnostic group revealed that 53 of the 108 metabolites showing significant sex-differences were also significant in each of the three groups (AD, MCI, CN) alone, while 14 metabolites showed no significant difference in any of the groups, probably due to lower statistical power after stratification (Table 4 and FIG. 4). Significant sex-differences limited to one diagnostic group were found for 8 metabolites (PC aa C34:1, PC ae C34:3, PC ae C36:3, PC ae C36:4, PC ae C38:5, PC ae C40:5, Histidine, C6/C4:1-DC) in patients with probable AD, for 7 metabolites (CO, C3, C9, C18:2, SDMA, Spermidine, t4-OH-Pro) in the MCI group, and for 6 metabolites (PC aa C42:0, PC ae C32:1, PC ae C42:3, SM(OH) C24:1, Sarcosine, Aspartate) in the CN group, although no significant sex-differences were found that were not also significant in the full cohort. Comparisons of beta estimates for sex between AD and CN groups showed no significant effect heterogeneity, indicating reduced power as source for these observed differences. Only PC aa C34:1 showed significant (p=0.029) heterogeneity between AD patients compared to CN subjects. Interestingly, in the larger healthy cohort used as reference, sex did not significantly affect the blood level of this metabolite when adjusting for the same covariates as in this study (i.e., age and BMI) [24]. In summary, we found that sex differences of blood metabolite levels are consistent (if we neglect the reduced power due to stratification) across diagnostic groups and, thus, do not seem to be directly affected by presence of MCI or AD status.

TABLE 4
Metabolic imprint of sex in ADNI cohorts for all participants and stratified by diagnostic groups
(CN, LMCI, and probable AD). Additionally, the results from the Mittelstrass et al. study in the
population-based KORA cohort are given

| | | ADNI subjects (n = 1.517) | | |
|---|---|---|---|---|
| | | | Abs. Diff. | |
| Metabolite | Pathway | Levels higher in | (mean) | p-value |
| Creatinine | Biogenic Amines | males | 0.891 | 1.54E−73 |
| SM C16:1 | Sphingolipids | females | 0.877 | 4.75E−72 |
| SM C18:1 | Sphingolipids | females | 0.864 | 9.73E−70 |
| PC aa C32:3 | Glycerophospholipids | females | 0.865 | 1.23E−69 |
| SM (OH) C22:2 | Sphingolipids | females | 0.844 | 7.08E−66 |
| SM C20:2 | Sphingolipids | females | 0.796 | 3.17E−58 |
| PC ae C30:2 | Glycerophospholipids | females | 0.790 | 4.72E−57 |
| PC aa C34:3 | Glycerophospholipids | females | 0.783 | 1.46E−55 |
| PC aa C38:3 | Glycerophospholipids | females | 0.753 | 3.14E−52 |
| PC aa C34:4 | Glycerophospholipids | females | 0.722 | 1.82E−47 |
| SM C18:0 | Sphingolipids | females | 0.711 | 3.39E−46 |
| Isoleucine | Amino Acids | males | 0.708 | 5.50E−45 |
| PC aa C38:5 | Glycerophospholipids | females | 0.692 | 1.35E−43 |
| PC ae C38:0 | Glycerophospholipids | females | 0.674 | 4.96E−41 |
| PC aa C36.6 | Glycerophospholipids | females | 0.654 | 9.14E−39 |
| PC ae C32:2 | Glycerophospholipids | females | 0.648 | 8.51E−38 |
| PC aa C40:5 | Glycerophospholipids | females | 0.634 | 9.51E−37 |
| SM (OH) C22:1 | Sphingolipids | females | 0.634 | 1.62E−36 |
| PC aa C38:4 | Glycerophospholipids | females | 0.617 | 5.87E−35 |
| PC aa C40:6 | Glycerophospholipids | females | 0.619 | 1.59E−34 |
| lysoPC a C28:1 | Glycerophospholipids | females | 0.614 | 7.14E−34 |
| PC aa C28:1 | Glycerophospholipids | females | 0.588 | 1.84E−31 |
| PC ae C42:1 | Glycerophospholipids | females | 0.587 | 6.86E−31 |
| PC aa C42:6 | Glycerophospholipids | females | 0.582 | 2.47E−30 |
| PC aa C36:1 | Glycerophospholipids | females | 0.579 | 3.09E−30 |
| PC aa C32:1 | Glycerophospholipids | females | 0.570 | 1.48E−29 |
| PC ae C40:3 | Glycerophospholipids | females | 0.553 | 1.31E−27 |
| SM C24:1 | Sphingolipids | females | 0.539 | 1.44E−26 |
| PC aa C42:5 | Glycerophospholipids | females | 0.540 | 1.84E−26 |
| Methionine | Amino Acids | males | 0.533 | 5.91E−26 |
| SM C16:0 | Sphingolipids | females | 0.529 | 9.52E−26 |
| PC aa C40:3 | Glycerophospholipids | females | 0.529 | 1.75E−25 |
| PC ae C38:6 | Glycerophospholipids | females | 0.525 | 3.23E−25 |
| PC ae C38:3 | Glycerophospholipids | females | 0.514 | 7.17E−24 |
| SM (OH) C16:1 | Sphingolipids | females | 0.505 | 3.27E−23 |
| PC ae C42:2 | Glycerophospholipids | females | 0.502 | 7.10E−23 |
| PC aa C30:0 | Glycerophospholipids | females | 0.497 | 2.13E−22 |
| PC ae C34:1 | Glycerophospholipids | females | 0.499 | 2.39E−22 |
| PC aa C38:6 | Glycerophospholipids | females | 0.491 | 5.51E−22 |
| Valine | Amino Acids | males | 0.492 | 6.16E−22 |
| PC ae C36:1 | Glycerophospholipids | females | 0.492 | 7.84E−22 |
| SM (OH) C14:1 | Sphingolipids | females | 0.479 | 5.93E−21 |
| Glycine | Amino Acids | females | 0.490 | 6.24E−21 |
| Proline | Amino Acids | males | 0.475 | 1.37E−20 |
| PC ae C40:2 | Glycerophospholipids | females | 0.474 | 1.62E−20 |
| PC aa C38:0 | Glycerophospholipids | females | 0.469 | 3.71E−20 |
| PC ae C40:1 | Glycerophospholipids | females | 0.457 | 3.62E−19 |
| PC aa C36:3 | Glycerophospholipids | females | 0.451 | 1.16E−18 |
| PC aa C36:5 | Glycerophospholipids | females | 0.448 | 1.48E−18 |
| Tryptophan | Amino Acids | males | 0.446 | 3.94E−18 |
| PC aa C40:4 | Glycerophospholipids | females | 0.439 | 5.51E−18 |
| PC ae C40:6 | Glycerophospholipids | females | 0.425 | 9.61E−17 |
| PC aa C32:0 | Glycerophospholipids | females | 0.422 | 2.58E−16 |
| lysoPC a C16:1 | Glycerophospholipids | females | 0.409 | 1.74E−15 |
| PC aa C36:4 | Glycerophospholipids | females | 0.401 | 2.36E−15 |
| PC ae C44:3 | Glycerophospholipids | females | 0.404 | 3.52E−15 |
| PC aa C24:0 | Glycerophospholipids | females | 0.400 | 5.62E−15 |
| C5 | Acylcarnitines | males | 0.395 | 1.39E−14 |
| lysoPC a C18:2 | Glycerophospholipids | males | 0.385 | 6.26E−14 |
| PC ae C34:0 | Glycerophospholipids | females | 0.373 | 4.52E−13 |
| PC aa C34:1 | Glycerophospholipids | females | 0.359 | 2.62E−12 |
| C14:1 | Acylcarnitines | females | 0.357 | 2.93E−12 |
| PC aa C42:4 | Glycerophospholipids | females | 0.357 | 3.39E−12 |
| PC ae C34:2 | Glycerophospholipids | females | 0.355 | 5.03E−12 |
| C16:1 | Acylcarnitines | females | 0.352 | 6.81E−12 |
| PC ae C36:2 | Glycerophospholipids | females | 0.349 | 1.44E−11 |
| SM (OH) C24:1 | Sphingolipids | females | 0.342 | 2.60E−11 |
| SM C24:0 | Sphingolipids | females | 0.340 | 2.69E−11 |
| PC ae C42:3 | Glycerophospholipids | females | 0.342 | 2.84E−11 |
| PC aa C42:1 | Glycerophospholipids | females | 0.339 | 3.77E−11 |
| C6 (C4:1-DC) | Acylcarnitines | females | 0.330 | 1.02E−10 |
| C18 | Acylcarnitines | males | 0.331 | 1.07E−10 |
| PC ae C36:3 | Glycerophospholipids | females | 0.330 | 1.31E−10 |
| PC aa C42:0 | Glycerophospholipids | females | 0.326 | 2.03E−10 |
| PC aa C40:2 | Glycerophospholipids | females | 0.322 | 3.29E−10 |
| PC ae C32:1 | Glycerophospholipids | females | 0.318 | 6.50E−10 |
| Kynurenine | Biogenic Amines | males | 0.313 | 9.57E−10 |
| PC ae C36:0 | Glycerophospholipids | females | 0.306 | 2.48E−09 |
| PC ae C30:0 | Glycerophospholipids | females | 0.306 | 3.20E−09 |
| C3 | Acylcarnitines | males | 0.299 | 6.12E−09 |
| Histidine | Amino Acids | males | 0.294 | 1.01E−08 |
| SM C26:1 | Sphingolipids | females | 0.293 | 1.32E−08 |
| PC ae C40:5 | Glycerophospholipids | females | 0.290 | 1.53E−08 |
| SM C26:0 | Sphingolipids | females | 0.289 | 2.10E−08 |
| PC ae C36:5 | Glycerophospholipids | females | 0.285 | 2.73E−08 |
| Asparagine | Amino Acids | males | 0.286 | 2.78E−08 |
| PC ae C34:3 | Glycerophospholipids | females | 0.283 | 3.70E−08 |
| Sarcosine | Biogenic Amines | males | 0.281 | 5.25E−08 |
| Aspartate | Amino Acids | females | 0.264 | 3.00E−07 |
| PC ae C38:4 | Glycerophospholipids | females | 0.261 | 3.77E−07 |
| C0 | Acylcarnitines | males | 0.246 | 1.52E−06 |
| PC ae C38:5 | Glycerophospholipids | females | 0.246 | 1.68E−06 |
| T4-OH-Pro | Biogenic Amines | males | 0.245 | 2.48E−06 |
| C5-DC (C6-OH) | Acylcarnitines | females | 0.232 | 6.28E−06 |
| Taurine | Biogenic Amines | females | 0.229 | 9.78E−06 |
| Spermidine | Biogenic Amines | males | 0.226 | 1.06E−05 |
| lysoPC a C24:0 | Glycerophospholipids | females | 0.226 | 1.13E−05 |
| PC ae C40:4 | Glycerophospholipids | females | 0.223 | 1.37E−05 |
| PC ae C36:4 | Glycerophospholipids | females | 0.219 | 2.00E−05 |
| PC aa C42:2 | Glycerophospholipids | females | 0.219 | 2.01E−05 |
| SDMA | Biogenic Amines | males | 0.219 | 2.06E−05 |
| C7-DC | Acylcarnitines | males | 0.203 | 7.43E−05 |
| C9 | Acylcarnitines | males | 0.198 | 1.26E−04 |
| Threonine | Amino Acids | males | 0.192 | 2.23E−04 |
| Serine | Amino Acids | females | 0.188 | 2.85E−04 |
| C18:2 | Acylcarnitines | males | 0.176 | 6.18E−04 |
| Tyrosine | Amino Acids | males | 0.176 | 6.52E−04 |
| PC ae C42:5 | Glycerophospholipids | females | 0.173 | 7.93E−04 |

| CN (n = 362) | | | | LMCI (n = 490) | | | |
|---|---|---|---|---|---|---|---|
| Levels | Abs. Diff. | | | Levels | Abs. Duff. | | |
| higher in | (mean) | p-value | power | higher in | (mean) | p-value | power |
| males | 0.954 | 4.43E−22 | 1.000 | males | 0.921 | 3.33E−25 | 1.000 |
| females | 0.875 | 2.70E−18 | 1.000 | females | 0.755 | 1.39E−16 | 1.000 |
| females | 0.883 | 1.33E−18 | 1.000 | females | 0.767 | 1.19E−17 | 1.000 |
| females | 0.914 | 4.74E−20 | 1.000 | females | 0.761 | 2.07E−17 | 1.000 |
| females | 0.872 | 2.55E−18 | 1.000 | females | 0.754 | 5.20E−17 | 1.000 |
| females | 0.830 | 1.82E−16 | 1.000 | females | 0.829 | 1.22E−20 | 1.000 |
| females | 0.838 | 7.43E−17 | 1.000 | females | 0.686 | 2.75E−14 | 1.000 |
| females | 0.795 | 3.59E−15 | 1.000 | females | 0.718 | 1.26E−14 | 1.000 |
| females | 0.755 | 1.02E−13 | 1.000 | females | 0.744 | 5.16E−17 | 1.000 |
| females | 0.800 | 2.09E−15 | 1.000 | females | 0.688 | 4.69E−14 | 1.000 |
| females | 0.733 | 6.11E−13 | 1.000 | females | 0.729 | 4.49E−16 | 1.000 |
| males | 0.874 | 2.39E−18 | 1.000 | males | 0.697 | 6.63E−14 | 1.000 |
| females | 0.747 | 2.00E−13 | 0.999 | females | 0.622 | 1.23E−11 | 1.000 |
| females | 0.811 | 9.08E−16 | 0.999 | females | 0.632 | 5.29E−12 | 1.000 |
| females | 0.786 | 7.77E−15 | 0.998 | females | 0.631 | 4.98E−12 | 1.000 |
| females | 0.677 | 3.62E−11 | 0.997 | females | 0.567 | 1.20E−09 | 1.000 |
| females | 0.651 | 2.38E−10 | 0.996 | females | 0.618 | 5.12E−12 | 1.000 |
| females | 0.592 | 8.58E−09 | 0.996 | females | 0.601 | 2.21E−11 | 1.000 |
| females | 0.604 | 4.54E−09 | 0.994 | females | 0.533 | 3.17E−09 | 1.000 |
| females | 0.694 | 1.06E−11 | 0.994 | females | 0.649 | 1.12E−12 | 1.000 |
| females | 0.695 | 9.08E−12 | 0.993 | females | 0.541 | 2.10E−09 | 0.999 |
| females | 0.605 | 4.00E−09 | 0.987 | females | 0.607 | 8.56E−12 | 0.999 |
| females | 0.576 | 2.24E−08 | 0.987 | females | 0.549 | 3.68E−09 | 0.999 |
| females | 0.549 | 1.06E−07 | 0.985 | females | 0.596 | 9.62E−11 | 0.998 |
| females | 0.489 | 2.49E−06 | 0.984 | females | 0.584 | 3.12E−10 | 0.998 |
| females | 0.532 | 2.76E−07 | 0.980 | females | 0.627 | 4.48E−12 | 0.997 |
| females | 0.632 | 7.48E−10 | 0.972 | females | 0.512 | 4.01E−08 | 0.996 |
| females | 0.539 | 1.88E−07 | 0.962 | females | 0.524 | 1.46E−08 | 0.993 |
| females | 0.509 | 9.18E−07 | 0.962 | females | 0.620 | 6.92E−12 | 0.993 |
| males | 0.679 | 3.25E−11 | 0.957 | males | 0.449 | 8.82E−07 | 0.992 |
| females | 0.517 | 6.74E−07 | 0.953 | females | 0.475 | 2.30E−07 | 0.991 |
| females | 0.476 | 4.60E−06 | 0.953 | females | 0.539 | 5.71E−09 | 0.991 |
| females | 0.605 | 4.24E−09 | 0.949 | females | 0.387 | 2.23E−05 | 0.990 |
| females | 0.549 | 9.83E−08 | 0.937 | females | 0.435 | 2.93E−06 | 0.986 |
| females | 0.578 | 1.99E−08 | 0.927 | females | 0.445 | 1.10E−06 | 0.983 |
| females | 0.508 | 9.84E−07 | 0.922 | females | 0.480 | 3.12E−07 | 0.981 |
| females | 0.514 | 6.60E−07 | 0.916 | females | 0.586 | 1.70E−10 | 0.979 |
| females | 0.483 | 3.09E−06 | 0.918 | females | 0.488 | 2.29E−07 | 0.979 |
| females | 0.584 | 1.43E−08 | 0.906 | females | 0.477 | 2.82E−07 | 0.975 |
| males | 0.564 | 4.65E−08 | 0.907 | males | 0.476 | 2.47E−07 | 0.975 |
| females | 0.476 | 4.50E−06 | 0.908 | females | 0.468 | 6.32E−07 | 0.975 |
| females | 0.537 | 2.02E−07 | 0.885 | females | 0.447 | 7.55E−07 | 0.966 |
| females | 0.473 | 4.83E−06 | 0.904 | females | 0.525 | 6.40E−08 | 0.974 |
| males | 0.605 | 3.99E−09 | 0.878 | males | 0.377 | 4.46E−05 | 0.963 |
| females | 0.556 | 7.45E−08 | 0.877 | females | 0.432 | 3.72E−06 | 0.962 |
| females | 0.553 | 8.66E−08 | 0.866 | females | 0.393 | 2.07E−05 | 0.957 |
| females | 0.513 | 7.72E−07 | 0.841 | females | 0.374 | 7.06E−05 | 0.945 |
| females | 0.319 | 2.34E−03 | 0.826 | females | 0.385 | 5.55E−05 | 0.937 |
| females | 0.518 | 5.69E−07 | 0.818 | females | 0.451 | 9.87E−07 | 0.932 |
| males | 0.461 | 9.09E−06 | 0.814 | males | 0.351 | 2.22E−04 | 0.930 |
| females | 0.394 | 1.62E−04 | 0.797 | females | 0.400 | 1.37E−05 | 0.920 |
| females | 0.564 | 4.84E−08 | 0.757 | females | 0.329 | 4.47E−04 | 0.895 |
| females | 0.410 | 8.33E−05 | 0.746 | females | 0.451 | 1.77E−06 | 0.887 |
| females | 0.386 | 2.12E−04 | 0.706 | females | 0.450 | 8.98E−07 | 0.859 |
| females | 0.281 | 7.39E−03 | 0.681 | females | 0.351 | 1.71E−04 | 0.840 |
| females | 0.431 | 3.57E−05 | 0.689 | females | 0.430 | 3.70E−06 | 0.847 |
| females | 0.493 | 1.99E−06 | 0.677 | females | 0.315 | 7.40E−04 | 0.837 |
| males | 0.480 | 3.64E−06 | 0.661 | males | 0.486 | 1.80E−07 | 0.824 |
| males | 0.478 | 4.16E−06 | 0.624 | males | 0.391 | 2.81E−05 | 0.794 |
| females | 0.366 | 4.60E−04 | 0.581 | females | 0.373 | 8.69E−05 | 0.756 |
| females | 0.188 | 7.40E−02 | 0.528 | females | 0.301 | 1.71E−03 | 0.705 |
| females | 0.385 | 2.25E−04 | 0.519 | females | 0.333 | 2.79E−04 | 0.696 |
| females | 0.320 | 2.22E−03 | 0.520 | females | 0.319 | 6.97E−04 | 0.697 |
| females | 0.367 | 4.46E−04 | 0.513 | females | 0.249 | 8.03E−03 | 0.690 |
| females | 0.455 | 1.18E−05 | 0.502 | females | 0.315 | 7.22E−04 | 0.679 |
| females | 0.368 | 4.24E−04 | 0.491 | females | 0.223 | 2.04E−02 | 0.668 |
| females | 0.363 | 5.03E−04 | 0.464 | females | 0.289 | 1.74E−03 | 0.640 |
| females | 0.231 | 2.78E−02 | 0.458 | females | 0.320 | 4.52E−04 | 0.633 |
| females | 0.363 | 5.09E−04 | 0.465 | females | 0.303 | 1.31E−03 | 0.640 |
| females | 0.418 | 6.26E−05 | 0.454 | females | 0.278 | 3.31E−03 | 0.629 |
| females | 0.186 | 7.79E−02 | 0.420 | females | 0.241 | 7.33E−03 | 0.590 |
| males | 0.165 | 1.16E−01 | 0.422 | males | 0.436 | 2.45E−06 | 0.593 |
| females | 0.310 | 3.06E−03 | 0.419 | females | 0.173 | 6.32E−02 | 0.590 |
| females | 0.425 | 4.77E−05 | 0.405 | females | 0.292 | 1.76E−03 | 0.574 |
| females | 0.273 | 9.49E−03 | 0.391 | females | 0.328 | 5.39E−04 | 0.558 |
| females | 0.359 | 5.96E−04 | 0.376 | females | 0.288 | 2.26E−03 | 0.540 |
| males | 0.476 | 4.79E−06 | 0.359 | males | 0.244 | 9.72E−03 | 0.519 |
| females | 0.211 | 4.47E−02 | 0.334 | females | 0.413 | 1.09E−05 | 0.489 |
| females | 0.367 | 4.35E−04 | 0.333 | females | 0.379 | 5.05E−05 | 0.487 |
| males | 0.277 | 8.36E−03 | 0.310 | males | 0.428 | 3.47E−06 | 0.458 |
| males | 0.290 | 5.62E−03 | 0.292 | males | 0.286 | 1.88E−03 | 0.435 |
| females | 0.301 | 4.02E−03 | 0.289 | females | 0.307 | 1.20E−03 | 0.432 |
| females | 0.339 | 1.24E−03 | 0.281 | females | 0.190 | 4.09E−02 | 0.420 |
| females | 0.330 | 1.62E−03 | 0.278 | females | 0.265 | 4.87E−03 | 0.416 |
| females | 0.357 | 6.36E−04 | 0.265 | females | 0.078 | 3.99E−01 | 0.399 |
| males | 0.330 | 1.57E−03 | 0.269 | males | 0.205 | 2.77E−02 | 0.405 |
| females | 0.312 | 2.88E−03 | 0.258 | females | 0.113 | 2.26E−01 | 0.389 |
| males | 0.353 | 7.40E−04 | 0.254 | males | 0.261 | 4.73E−03 | 0.384 |
| females | 0.363 | 5.27E−04 | 0.205 | females | 0.149 | 1.03E−01 | 0.316 |
| females | 0.319 | 2.28E−03 | 0.196 | females | 0.077 | 4.01E−01 | 0.303 |
| males | 0.125 | 2.37E−01 | 0.160 | males | 0.449 | 1.30E−06 | 0.250 |
| females | 0.282 | 7.12E−03 | 0.160 | females | 0.053 | 5.68E−01 | 0.250 |
| males | 0.243 | 2.03E−02 | 0.157 | males | 0.427 | 6.19E−06 | 0.246 |
| females | 0.249 | 1.72E−02 | 0.129 | females | 0.047 | 6.14E−01 | 0.204 |
| females | 0.154 | 1.43E−01 | 0.123 | females | 0.289 | 1.89E−03 | 0.194 |
| males | 0.091 | 3.88E−01 | 0.119 | males | 0.407 | 6.54E−06 | 0.188 |
| females | 0.146 | 1.66E−01 | 0.119 | females | 0.169 | 7.37E−02 | 0.188 |
| females | 0.225 | 3.21E−02 | 0.114 | females | 0.128 | 1.73E−01 | 0.180 |
| females | 0.222 | 3.44E−02 | 0.105 | females | 0.014 | 8.76E−01 | 0.167 |
| females | 0.110 | 2.98E−01 | 0.106 | females | 0.253 | 7.40E−03 | 0.168 |
| males | 0.244 | 2.04E−02 | 0.105 | males | 0.377 | 4.29E−05 | 0.167 |
| males | 0.166 | 1.16E−01 | 0.080 | males | 0.260 | 4.63E−03 | 0.127 |
| males | 0.145 | 1.66E−01 | 0.074 | males | 0.332 | 3.40E−04 | 0.117 |
| males | 0.288 | 5.90E−03 | 0.067 | males | 0.140 | 1.43E−01 | 0.105 |
| females | 0.244 | 2.01E−02 | 0.061 | females | 0.205 | 2.97E−02 | 0.097 |
| males | 0.060 | 5.66E−01 | 0.049 | males | 0.356 | 1.58E−04 | 0.076 |
| males | 0.181 | 8.54E−02 | 0.049 | males | 0.062 | 5.08E−01 | 0.077 |
| females | 0.235 | 2.57E−02 | 0.046 | females | 0.119 | 2.04E−01 | 0.072 |

| AD (n = 302) | | | | | | |
|---|---|---|---|---|---|---|
| Levels | Abs. Diff. | | | difference between CN and AD | | |
| higher in | (mean) | p-value | power | Diff t | Diff p-value | Diff I² |
| males | 0.806 | 5.18E−13 | 1.000 | −1.048 | 2.95E−01 | 4.604 |
| females | 0.955 | 1.00E−18 | 1.000 | −0.579 | 5.63E−01 | 0.000 |
| females | 1.007 | 1.68E−20 | 1.000 | −0.899 | 3.69E−01 | 0.000 |
| females | 0.929 | 2.78E−17 | 1.000 | −0.109 | 9.13E−01 | 0.000 |
| females | 0.833 | 3.53E−14 | 1.000 | 0.282 | 7.78E−01 | 0.000 |
| females | 0.876 | 2.54E−15 | 1.000 | −0.329 | 7.42E−01 | 0.000 |
| females | 0.794 | 9.57E−13 | 1.000 | 0.308 | 7.58E−01 | 0.000 |
| females | 0.870 | 7.76E−15 | 1.000 | −0.527 | 5.98E−01 | 0.000 |
| lemales | 0.837 | 2.71E−14 | 0.999 | −0.576 | 5.64E−01 | 0.000 |
| females | 0.682 | 1.82E−09 | 0.998 | 0.812 | 4.17E−01 | 0.000 |
| females | 0.795 | 1.00E−12 | 0.997 | −0.432 | 6.66E−01 | 0.000 |
| males | 0.644 | 1.55E−08 | 0.997 | −1.579 | 1.14E−01 | 36.680 |
| females | 0.757 | 1.22E−11 | 0.996 | −0.064 | 9.49E−01 | 0.000 |
| females | 0.711 | 2.61E−10 | 0.993 | 0.691 | 4.90E−01 | 0.000 |
| females | 0.605 | 1.01E−07 | 0.989 | 1.231 | 2.19E−01 | 18.732 |
| lemales | 0.769 | 5.22E−12 | 0.988 | −0.632 | 5.27E−01 | 0.000 |
| females | 0.704 | 4.53E−10 | 0.983 | −0.356 | 7.22E−01 | 0.000 |
| females | 0.695 | 4.74E−10 | 0.983 | −0.703 | 4.82E−01 | 0.000 |
| fernales | 0.757 | 5.40E−12 | 0.976 | −1.047 | 2.95E−01 | 4.458 |
| females | 0.651 | 7.90E−09 | 0.977 | 0.291 | 7.71E−01 | 0.000 |
| females | 0.514 | 8.49E−06 | 0.974 | 1.208 | 2.27E−01 | 17.239 |
| females | 0.502 | 1.08E−05 | 0.958 | 0.686 | 4.93E−01 | 0.000 |
| females | 0.662 | 5.14E−09 | 0.956 | −0.576 | 5.64E−01 | 0.000 |
| females | 0.612 | 7.18E−08 | 0.952 | −0.418 | 6.76E−01 | 0.000 |
| females | 0.632 | 2.73E−08 | 0.950 | −0.947 | 3.43E−01 | 0.000 |
| females | 0.548 | 1.44E−06 | 0.941 | −0.104 | 9.17E−01 | 0.000 |
| lemales | 0.493 | 1.56E−05 | 0.923 | 0.923 | 3.56E−01 | 0.000 |
| females | 0.586 | 1.85E−07 | 0.904 | −0.312 | 7.55E−01 | 0.000 |
| females | 0.615 | 4.91E−08 | 0.905 | −0.707 | 4.79E−01 | 0.000 |
| males | 0.592 | 2.22E−07 | 0.895 | −0.584 | 5.59E−01 | 0.000 |
| females | 0.545 | 1.40E−06 | 0.888 | −0.187 | 8.52E−01 | 0.000 |
| females | 0.643 | 1.02E−08 | 0.889 | −1.117 | 2.64E−01 | 10.505 |
| females | 0.722 | 6.48E−11 | 0.881 | −0.797 | 4.25E−01 | 0.000 |
| lemales | 0.554 | 1.56E−06 | 0.862 | −0.027 | 9.78E−01 | 0.000 |
| females | 0.487 | 2.38E−05 | 0.845 | 0.599 | 5.49E−01 | 0.000 |
| females | 0.550 | 1.67E−06 | 0.838 | −0.277 | 7.82E−01 | 0.000 |
| females | 0.385 | 9.00E−04 | 0.828 | 0.843 | 3.99E−01 | 0.000 |
| females | 0.537 | 2.68E−06 | 0.831 | −0.355 | 7.23E−01 | 0.000 |
| females | 0.545 | 1.46E−06 | 0.813 | 0.263 | 7.92E−01 | 0.000 |
| males | 0.560 | 9.61E−07 | 0.816 | −0.025 | 9.80E−01 | 0.000 |
| females | 0.512 | 9.53E−06 | 0.816 | −0.238 | 8.12E−01 | 0.000 |
| females | 0.398 | 5.72E−04 | 0.784 | 0.910 | 3.63E−01 | 0.000 |
| females | 0.436 | 2.11E−04 | 0.810 | 0.243 | 8.08E−01 | 0.000 |
| males | 0.451 | 9.10E−05 | 0.774 | −1.021 | 3.07E−01 | 2.065 |
| females | 0.441 | 1.14E−04 | 0.773 | 0.760 | 4.47E−01 | 0.000 |
| females | 0.616 | 3.91E−08 | 0.759 | −0.424 | 6.71E−01 | 0.000 |
| females | 0.524 | 5.49E−06 | 0.727 | −0.076 | 9.40E−01 | 0.000 |
| females | 0.575 | 4.91E−07 | 0.709 | −1.676 | 9.38E−02 | 40.324 |
| females | 0.439 | 1.25E−04 | 0.699 | 0.523 | 6.01E−01 | 0.000 |
| males | 0.718 | 2.72E−10 | 0.694 | 1.714 | 8.65E−02 | 41.666 |
| lemales | 0.520 | 4.87E−06 | 0.673 | −0.832 | 4.05E−01 | 0.000 |
| females | 0.505 | 8.18E−06 | 0.629 | 0.391 | 6.96E−01 | 0.000 |
| females | 0.521 | 5.82E−06 | 0.617 | −0.731 | 4.65E−01 | 0.000 |
| females | 0.381 | 1.13E−03 | 0.574 | 0.032 | 9.75E−01 | 0.000 |
| females | 0.570 | 3.48E−07 | 0.549 | −1.908 | 5.64E−02 | 47.595 |
| females | 0.355 | 2.25E−03 | 0.557 | 0.492 | 6.23E−01 | 0.000 |
| females | 0.370 | 1.30E−03 | 0.544 | 0.809 | 4.18E−01 | 0.000 |
| males | 0.380 | 9.85E−04 | 0.529 | −0.654 | 5.13E−01 | 0.000 |
| males | 0.366 | 1.58E−03 | 0.493 | −0.730 | 4.65E−01 | 0.000 |
| females | 0.357 | 2.21E−03 | 0.453 | 0.059 | 9.53E−01 | 0.000 |
| females | 0.522 | 4.19E−06 | 0.405 | −2.187 | 2.87E−02 | 54.282 |
| females | 0.441 | 1.12E−04 | 0.397 | −0.372 | 7.10E−01 | 0.000 |
| females | 0.460 | 5.36E−05 | 0.398 | −0.911 | 3.62E−01 | 0.000 |
| fernales | 0.426 | 2.16E−04 | 0.392 | −0.384 | 7.01E−01 | 0.000 |
| females | 0.376 | 1.11E−03 | 0.382 | 0.517 | 6.05E−01 | 0.000 |
| females | 0.425 | 2.48E−04 | 0.373 | −0.368 | 7.13E−01 | 0.000 |
| females | 0.368 | 1.43E−03 | 0.350 | −0.031 | 9.75E−01 | 0.000 |
| females | 0.468 | 3.65E−05 | 0.345 | −1.550 | 1.21E−01 | 35.468 |
| females | 0.351 | 2.37E−03 | 0.350 | 0.078 | 9.38E−01 | 0.000 |
| females | 0.421 | 2.06E−04 | 0.341 | −0.020 | 9.84E−01 | 0.000 |
| females | 0.411 | 4.11E−04 | 0.312 | −1.451 | 1.47E−01 | 31.070 |
| males | 0.435 | 1.46E−04 | 0.315 | 1.748 | 8.04E−02 | 42.808 |
| females | 0.486 | 2.26E−05 | 0.312 | −1.148 | 2.51E−01 | 12.883 |
| females | 0.334 | 3.48E−03 | 0.301 | 0.594 | 5.53E−01 | 0.000 |
| females | 0.493 | 1.38E−05 | 0.290 | −1.439 | 1.50E−01 | 30.487 |
| females | 0.369 | 1.32E−03 | 0.277 | −0.067 | 9.47E−01 | 0.000 |
| males | 0.419 | 2.71E−04 | 0.264 | −0.374 | 7.09E−01 | 0.000 |
| females | 0.415 | 3.15E−04 | 0.244 | −1.318 | 1.88E−01 | 24.124 |
| females | 0.098 | 3.98E−01 | 0.243 | 1.727 | 8.42E−02 | 42.090 |
| males | 0.265 | 2.20E−02 | 0.225 | −0.079 | 9.37E−01 | 0.000 |
| males | 0.493 | 1.74E−05 | 0.212 | 1.322 | 1.86E−01 | 24.384 |
| females | 0.358 | 1.96E−03 | 0.209 | −0.363 | 7.17E−01 | 0.000 |
| females | 0.396 | 5.44E−04 | 0.203 | −0.372 | 7.10E−01 | 0.000 |
| females | 0.299 | 9.66E−03 | 0.200 | 0.202 | 8.40E−01 | 0.000 |
| females | 0.504 | 1.07E−05 | 0.191 | −0.961 | 3.37E−01 | 0.000 |
| males | 0.352 | 2.20E−03 | 0.194 | 0.137 | 8.91E−01 | 0.000 |
| females | 0.430 | 1.82E−04 | 0.185 | −0.764 | 4.45E−01 | 0.000 |
| males | 0.108 | 3.59E−01 | 0.182 | −1.564 | 1.18E−01 | 36.073 |
| females | 0.323 | 5.92E−03 | 0.146 | 0.251 | 8.02E−01 | 0.000 |
| females | 0.382 | 9.29E−04 | 0.140 | −0.407 | 6.84E−01 | 0.000 |
| males | 0.229 | 4.43E−02 | 0.113 | 0.673 | 5.01E−01 | 0.000 |
| females | 0.519 | 4.46E−06 | 0.114 | −1.560 | 1.19E−01 | 35.908 |
| males | 0.206 | 7.64E−02 | 0.112 | −0.234 | 8.15E−01 | 0.000 |
| females | 0.255 | 2.48E−02 | 0.092 | −0.037 | 9.70E−01 | 0.000 |
| females | 0.154 | 1.87E−01 | 0.087 | −0.003 | 9.97E−01 | 0.000 |
| males | 0.140 | 2.25E−01 | 0.084 | 0.315 | 7.52E−01 | 0.000 |
| females | 0.270 | 2.05E−02 | 0.084 | −0.796 | 4.26E−01 | 0.000 |
| females | 0.246 | 3.29E−02 | 0.081 | −0.132 | 8.95E−01 | 0.000 |
| females | 0.467 | 4.70E−05 | 0.075 | −1.589 | 1.12E−01 | 37.075 |
| females | 0.376 | 1.13E−03 | 0.075 | −1.711 | 8.71E−02 | 41.559 |
| males | 0.120 | 2.96E−01 | 0.075 | −0.793 | 4.28E−01 | 0.000 |
| males | 0.081 | 4.81E−0l | 0.057 | −0.548 | 5.84E−01 | 0.000 |
| males | 0.223 | 5.51E−02 | 0.053 | 0.496 | 6.20E−01 | 0.000 |
| males | 0.190 | 1.06E−01 | 0.048 | −0.626 | 5.31E−01 | 0.000 |
| females | 0.109 | 3.50E−01 | 0.044 | 0.863 | 3.88E−01 | 0.000 |
| males | 0.108 | 3.46E−01 | 0.035 | 0.305 | 7.61E−01 | 0.000 |
| males | 0.332 | 4.34E−03 | 0.035 | 0.971 | 3.32E−01 | 0.000 |
| females | 0.176 | 1.26E−01 | 0.033 | 0.382 | 7.02E−01 | 0.000 |
| males | 0.806 | 5.18E−13 | 1.000 | −1.048 | 2.95E−01 | 4.604 |
| females | 0.955 | 1.00E−18 | 1.000 | −0.579 | 5.63E−01 | 0.000 |
| females | 1.007 | 1.68E−20 | 1.000 | −0.899 | 3.69E−01 | 0.000 |
| females | 0.929 | 2.78E−17 | 1.000 | −0.109 | 9.13E−01 | 0.000 |
| lemales | 0.833 | 3.53E−14 | 1.000 | 0.282 | 7.78E−01 | 0.000 |
| females | 0.876 | 2.54E−15 | 1.000 | −0.329 | 7.42E−01 | 0.000 |
| females | 0.794 | 9.57E−13 | 1.000 | 0.308 | 7.58E−01 | 0.000 |
| females | 0.870 | 7.76E−15 | 1.000 | −0.527 | 5.98E−01 | 0.000 |
| females | 0.837 | 2.71E−14 | 0.999 | −0.576 | 5.64E−01 | 0.000 |
| females | 0.682 | 1.82E−09 | 0.998 | 0.812 | 4.17E−01 | 0.000 |
| females | 0.795 | 1.00E−12 | 0.997 | −0.432 | 6.66E−01 | 0.000 |
| males | 0.644 | 1.55E−08 | 0.997 | −1.579 | 1.14E−01 | 36.680 |
| females | 0.757 | 1.22E−11 | 0.996 | -0.064 | 9.49E−01 | 0.000 |
| females | 0.711 | 2.61E−10 | 0.993 | 0.691 | 4.90E−01 | 0.000 |
| lemales | 0.605 | 1.01E−07 | 0.989 | 1.231 | 2.19E−01 | 18.732 |
| females | 0.769 | 5.22E−12 | 0.988 | −0.632 | 5.27E−01 | 0.000 |
| females | 0.704 | 4.53E−10 | 0.983 | −0.356 | 7.22E−01 | 0.000 |
| females | 0.695 | 4.74E−10 | 0.983 | −0.703 | 4.82E−01 | 0.000 |
| females | 0.757 | 5.40E−12 | 0.976 | −1.047 | 2.95E−01 | 4.458 |
| females | 0.651 | 7.90E−09 | 0.977 | 0.291 | 7.71E−01 | 0.000 |
| females | 0.514 | 8.49E−06 | 0.974 | 1.208 | 2.27E−01 | 17.239 |
| lemales | 0.502 | 1.08E−05 | 0.958 | 0.686 | 4.93E−01 | 0.000 |
| females | 0.662 | 5.14E−09 | 0.956 | −0.576 | 5.64E−01 | 0.000 |
| females | 0.612 | 7.18E−08 | 0.952 | −0.418 | 6.76E−01 | 0.000 |
| females | 0.632 | 2.73E−08 | 0.950 | −0.947 | 3.43E−01 | 0.000 |
| females | 0.548 | 1.44E−06 | 0.941 | −0.104 | 9.17E−01 | 0.000 |
| females | 0.493 | 1.56E−05 | 0.923 | 0.923 | 3.56E−01 | 0.000 |
| females | 0.586 | 1.85E−07 | 0.904 | −0.312 | 7.55E−01 | 0.000 |
| lemales | 0.615 | 4.91E−08 | 0.905 | −0.707 | 4.79E−01 | 0.000 |
| males | 0.592 | 2.22E−07 | 0.895 | −0.584 | 5.59E−01 | 0.000 |
| females | 0.545 | 1.40E−06 | 0.888 | −0.187 | 8.52E−01 | 0.000 |
| females | 0.643 | 1.02E−08 | 0.889 | −1.117 | 2.64E−01 | 10.505 |
| females | 0.722 | 6.48E−11 | 0.881 | −0.797 | 4.25E−01 | 0.000 |
| females | 0.554 | 1.56E−06 | 0.862 | −0.027 | 9.78E−01 | 0.000 |
| females | 0.487 | 2.38E−05 | 0.845 | 0.599 | 5.49E−01 | 0.000 |
| females | 0.550 | 1.67E−06 | 0.838 | −0.277 | 7.82E−01 | 0.000 |
| females | 0.385 | 9.00E−04 | 0.828 | 0.843 | 3.99E−01 | 0.000 |
| females | 0.537 | 2.68E−06 | 0.831 | −0.355 | 7.23E−01 | 0.000 |
| lemales | 0.545 | 1.46E−06 | 0.813 | 0.263 | 7.92E−01 | 0.000 |
| males | 0.560 | 9.61E−07 | 0.816 | −0.025 | 9.80E−01 | 0.000 |
| females | 0.512 | 9.53E−06 | 0.816 | −0.238 | 8.12E−01 | 0.000 |
| fernales | 0.398 | 5.72E−04 | 0.784 | 0.910 | 3.63E−01 | 0.000 |
| females | 0.436 | 2.11E−04 | 0.810 | 0.243 | 8.08E−01 | 0.000 |
| males | 0.451 | 9.10E−05 | 0.774 | −1.021 | 3.07E−01 | 2.065 |
| females | 0.441 | 1.14E−04 | 0.773 | 0.760 | 4.47E−01 | 0.000 |
| females | 0.616 | 3.91E−08 | 0.759 | −0.424 | 6.71E−01 | 0.000 |
| females | 0.524 | 5.49E−06 | 0.727 | −0.076 | 9.40E−01 | 0.000 |
| females | 0.575 | 4.91E−07 | 0.709 | −1.676 | 9.38E−02 | 40.324 |
| fernales | 0.439 | 1.25E−04 | 0.699 | 0.523 | 6.01E−01 | 0.000 |
| males | 0.718 | 2.72E−10 | 0.694 | 1.714 | 8.65E−02 | 41.666 |
| females | 0.520 | 4.87E−06 | 0.673 | −0.832 | 4.05E−01 | 0.000 |
| females | 0.505 | 8.18E−06 | 0.629 | 0.391 | 6.96E−01 | 0.000 |
| females | 0.521 | 5.82E−06 | 0.617 | −0.731 | 4.65E−01 | 0.000 |
| females | 0.381 | 1.13E−03 | 0.574 | 0.032 | 9.75E−01 | 0.000 |
| females | 0.570 | 3.48E−07 | 0.549 | −1.908 | 5.64E−02 | 47.595 |
| females | 0.355 | 2.25E−03 | 0.557 | 0.492 | 6.23E−01 | 0.000 |
| lemales | 0.370 | 1.30E−03 | 0.544 | 0.809 | 4.18E−01 | 0.000 |
| males | 0.380 | 9.85E−04 | 0.529 | −0.654 | 5.13E−01 | 0.000 |
| males | 0.366 | 1.58E−03 | 0.493 | −0.730 | 4.65E−01 | 0.000 |
| females | 0.357 | 2.21E−03 | 0.453 | 0.059 | 9.53E−01 | 0.000 |
| females | 0.522 | 4.19E−06 | 0.405 | −2.187 | 2.87E−02 | 54.282 |
| females | 0.441 | 1.12E−04 | 0.397 | −0.372 | 7.10E−01 | 0.000 |
| females | 0.460 | 5.36E−05 | 0.398 | −0.911 | 3.62E−01 | 0.000 |
| lemales | 0.426 | 2.16E−04 | 0.392 | −0.384 | 7.01E−01 | 0.000 |
| females | 0.376 | 1.11E−03 | 0.382 | 0.517 | 6.05E−01 | 0.000 |
| females | 0.425 | 2.48E−04 | 0.373 | −0.368 | 7.13E−01 | 0.000 |
| females | 0.368 | 1.43E−03 | 0.350 | −0.031 | 9.75E−01 | 0.000 |
| females | 0.468 | 3.65E−05 | 0.345 | −1.550 | 1.21E−01 | 35.468 |
| females | 0.351 | 2.37E−03 | 0.350 | 0.078 | 9.38E−01 | 0.000 |
| females | 0.421 | 2.06E−04 | 0.341 | −0.020 | 9.84E−01 | 0.000 |
| females | 0.411 | 4.11E−04 | 0.312 | −1.451 | 1.47E−01 | 31.070 |
| males | 0.435 | 1.46E−04 | 0.315 | 1.748 | 8.04E−02 | 42.808 |
| females | 0.486 | 2.26E−05 | 0.312 | −1.148 | 2.51E−01 | 12.883 |
| lemales | 0.334 | 3.48E−03 | 0.301 | 0.594 | 5.53E−01 | 0.000 |
| females | 0.493 | 1.38E−05 | 0.290 | −1.439 | 1.50E−01 | 30.487 |
| females | 0.369 | 1.32E−03 | 0.277 | −0.067 | 9.47E−01 | 0.000 |
| males | 0.419 | 2.71E−04 | 0.264 | −0.374 | 7.09E−01 | 0.000 |
| females | 0.415 | 3.15E−04 | 0.244 | −1.318 | 1.88E−01 | 24.124 |
| females | 0.098 | 3.98E−01 | 0.243 | 1.727 | 8.42E−02 | 42.090 |
| males | 0.265 | 2.20E−02 | 0.225 | −0.079 | 9.37E−01 | 0.000 |
| males | 0.493 | 1.74E−05 | 0.212 | 1.322 | 1.86E−01 | 24.384 |
| females | 0.358 | 1.96E−03 | 0.209 | −0.363 | 7.17E−01 | 0.000 |
| females | 0.396 | 5.44E−04 | 0.203 | −0.372 | 7.10E−01 | 0.000 |
| females | 0.299 | 9.66E−03 | 0.200 | 0.202 | 8.40E−01 | 0.000 |
| females | 0.504 | 1.07E−05 | 0.191 | −0.961 | 3.37E−01 | 0.000 |
| males | 0.352 | 2.20E−03 | 0.194 | 0.137 | 8.91E−01 | 0.000 |
| females | 0.430 | 1.82E−04 | 0.185 | −0.764 | 4.45E−01 | 0.000 |
| males | 0.108 | 3.59E−01 | 0.182 | −1.564 | 1.18E−01 | 36.073 |
| females | 0.323 | 5.92E−03 | 0.146 | 0.251 | 8.02E−01 | 0.000 |
| females | 0.382 | 9.29E−04 | 0.140 | −0.407 | 6.84E−01 | 0.000 |
| males | 0.229 | 4.43E−02 | 0.113 | 0.673 | 5.01E−01 | 0.000 |
| females | 0.519 | 4.46E−06 | 0.114 | −1.560 | 1.19E−01 | 35.908 |
| males | 0.206 | 7.64E−02 | 0.112 | −0.234 | 8.15E−01 | 0.000 |
| females | 0.255 | 2.48E−02 | 0.092 | −0.037 | 9.70E−01 | 0.000 |
| females | 0.154 | 1.87E−01 | 0.087 | −0.003 | 9.97E−01 | 0.000 |
| males | 0.140 | 2.25E−01 | 0.084 | 0.315 | 7.52E−01 | 0.000 |
| females | 0.270 | 2.05E−02 | 0.084 | −0.796 | 4.26E−01 | 0.000 |
| females | 0.246 | 3.29E−02 | 0.081 | −0.132 | 8.95E−01 | 0.000 |
| females | 0.467 | 4.70E−05 | 0.075 | −1.589 | 1.12E−01 | 37.075 |
| females | 0.376 | 1.13E−03 | 0.075 | −1.711 | 8.71E−02 | 41.559 |
| males | 0.120 | 2.96E−01 | 0.075 | −0.793 | 4.28E−01 | 0.000 |
| males | 0.081 | 4.81E−01 | 0.057 | −0.548 | 5.84E−01 | 0.000 |
| males | 0.223 | 5.51E−02 | 0.053 | 0.496 | 6.20E−01 | 0.000 |
| males | 0.190 | 1.06E−01 | 0.048 | −0.626 | 5.31E−01 | 0.000 |
| females | 0.109 | 3.50E−01 | 0.044 | 0.863 | 3.88E−01 | 0.000 |
| males | 0.108 | 3.46E−01 | 0.035 | 0.305 | 7.61E−01 | 0.000 |
| males | 0.332 | 4.34E−03 | 0.035 | 0.971 | 3.32E−01 | 0.000 |
| females | 0.176 | 1.26E−01 | 0.033 | 0.382 | 7.02E−01 | 0.000 |

KORA F4 (Mittelstrass et al.)

| ADM vs. KORA | Levels higher in | Effect | p-value |
|---|---|---|---|
| — | — | — | — |
| consistent | females | −0.2 | 3.50E−82 |
| consistent | females | −0.2 | 1.30E−101 |
| consistent | females | −0.2 | 1.40E−106 |
| consistent | females | −0.2 | 1.10E−124 |
| consistent | females | −0.3 | 7.50E−100 |
| consistent | females | −0.2 | 9.10E−53 |
| consistent | females | −0.1 | 2.70E−39 |
| consistent | females | −0.1 | 1.30E−12 |
| consistent | females | −0.1 | 1.40E−19 |
| consistent | females | −0.1 | 3.10E−36 |
| consistent | males | 0.2 | 1.60E−190 |
| — | (females) | (−0.03) | NS (1.9E−03) |
| consistent | females | −0.1 | 4.30E−22 |
| consistent | females | −0.1 | 5.60E−21 |
| consistent | females | −0.2 | 3.70E−90 |
| — | (females) | (−0.003) | NS (7.8E−01) |
| consistent | females | −0.1 | 2.40E−47 |
| consistent | females | −0.04 | 7.20E−05 |
| — | (females) | (−0.04) | NS (4.1E−04) |
| — | — | — | — |
| consistent | females | −0.1 | 1.10E−53 |
| — | (females) | (−0.03) | NS (6.5E−03) |
| consistent | females | −0.04 | 6.00E−07 |
| consistent | females | −0.04 | 4.50E−05 |
| consistent | females | −0.1 | 4.20E−08 |
| consistent | females | −0.2 | 5.00E−99 |
| consistent | females | −0.04 | 3.30E−06 |
| consistent | females | −0.04 | 6.00E−05 |
| consistent | males | 0.1 | 1.00E−36 |
| consistent | females | −0.1 | 120E−27 |
| — | (females) | (−0.03) | NS (7.5E−03) |
| consistent | females | −0.05 | 8.00E−08 |
| consistent | females | −0.1 | 7.40E−71 |
| consistent | females | −0.2 | 1.10E−84 |
| consistent | females | −0.1 | 1.50E−16 |
| consistent | females | −0.1 | 3.10E−21 |
| consistent | females | −0.1 | 5.10E−61 |
| consistent | females | −0.05 | 1.50E−04 |
| consistent | males | 0.1 | 1.90E−78 |
| consistent | females | −0.1 | 2.50E−51 |
| consistent | females | −0.2 | 9.30E−69 |
| consistent | females | −0.1 | 9.10E−46 |
| consistent | males | 0.2 | 1.20E−71 |
| consistent | females | −0.1 | 9.10E−21 |
| consistent | females | −0.1 | 2.90E−11 |
| — | (females) | (−0.02) | NS (3.8E−01) |
| consistent | females | −0.1 | 1.50E−11 |
| — | (males) | (0.005) | NS (7.9E−01) |
| consistent | males | 0.1 | 2.80E−36 |
| — | (females) | (−0.03) | NS (9.6E−01) |
| consistent | females | −0.1 | 2.40E−27 |
| — | (females) | (−0.03) | NS (1.7E−02) |
| — | (males) | (0.04) | NS (2.0E−03) |
| — | (females) | (−0.02) | NS (9.6E−02) |
| consistent | females | −0.1 | 4.80E−11 |
| — | — | — | — |
| consistent | males | 0.2 | 4.80E−85 |
| consistent | males | 0.2 | 6.20E−55 |
| consistent | females | −0.1 | 1.00E−29 |
| — | (females) | (−0.01) | NS (2.8E−02) |
| — | (males) | (0.03) | NS (4.2E−02) |
| consistent | females | −0.03 | 8.80E−06 |
| consistent | females | −0.1 | 4.90E−43 |
| — | (females) | (−0.01) | NS (3.9E−02) |
| consistent | females | −0.2 | 1.60E−79 |
| consistent | females | −0.1 | 5.30E−18 |
| — | (females) | (−0.01) | NS (1.7E−01) |
| consistent | females | −0.1 | 6.40E−29 |
| consistent | females | −0.1 | 4.40E−21 |
| — | — | — | — |
| consistent | males | 0.1 | 5.60E−57 |
| consistent | females | −0.1 | 2.20E−32 |
| consistent | females | −0.1 | 3.20E−31 |
| — | (females) | (−0.02) | NS (4.7E−02) |
| consistent | females | −0.1 | 3.40E−38 |
| — | — | — | — |
| — | — | — | — |
| consistent | females | −0.1 | 2.20E−41 |
| consistent | males | 0.2 | 3.50E−69 |
| consistent | males | 0.01 | 4.00E−07 |
| — | (females) | (−0.02) | NS (1.1E−01) |
| consistent | females | −0.1 | 1.30E−18 |
| — | — | — | — |
| — | (males) | (−0.01) | NS (4.3E−01) |
| — | — | — | |
| consistent | females | −0.1 | 1.80E−36 |
| — | — | — | — |
| — | — | — | — |
| consistent | females | −0.1 | 1.10E−15 |
| consistent | males | 0.1 | 1.60E−62 |
| — | (males) | (0.004) | NS (6.1E−01) |
| — | — | — | — |
| — | — | — | — |
| — | — | — | — |
| — | — | — | — |
| — | — | — | — |
| consistent | females | −0.1 | 3.90E−31 |
| — | (males) | (0.02) | NS (7.7E−02) |
| — | (females) | (−0.02) | NS (2.0E−01) |
| — | — | — | — |
| — | — | — | — |
| consistent | males | 0.1 | 7.80E−06 |
| — | (males) | (0.01) | NS (7.3E−02) |
| consistent | females | −0.1 | 5.30E−12 |
| consistent | males | 0.1 | 4.10E−42 |
| consistent | males | 0.1 | 4.60E−20 |
| consistent | females | −0.1 | 1.80E−23 |

Sex Modifies Associations of Metabolites with AD Biomarkers.

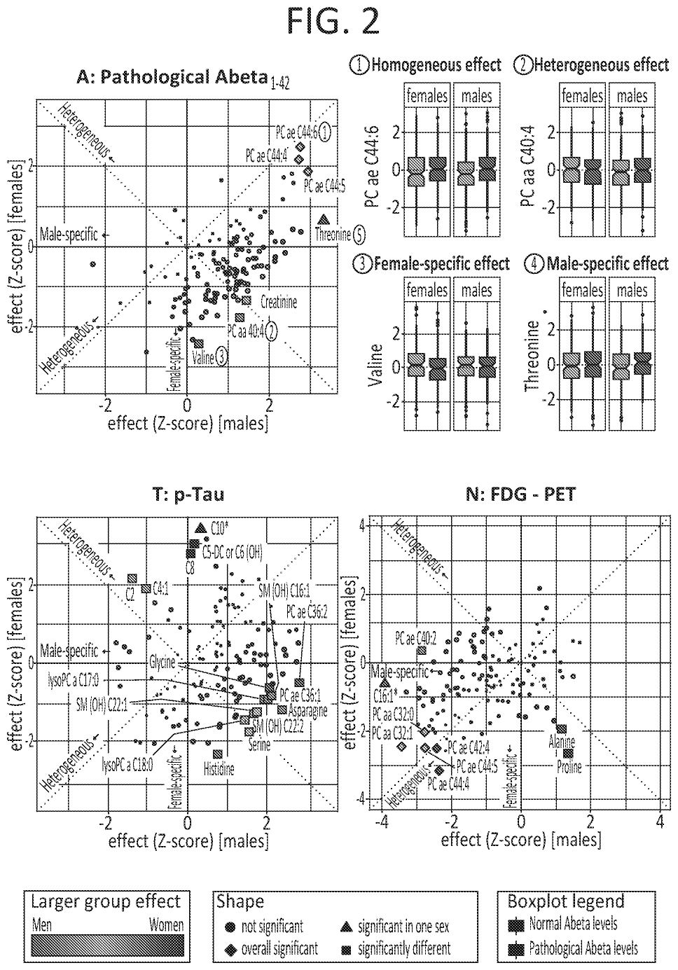

To investigate whether sex modifies the association between AD endophenotypes and metabolite concentrations, we tested for associations of the three representative A-T-N biomarkers, CSF Aβ₁₋₄₂ pathology, CSF p-tau levels, and brain glucose uptake measured via FDG-PET imaging, with concentrations of 140 blood metabolites. We did this in the full data set, as well as in women and men separately using multivariable linear and logistic regression, followed by analysis of heterogeneity of effects between sexes. Table 5 lists the results of these analyses for all metabolite-phenotype combinations, as well as analyses of sex-by-metabolite interaction effects on A-T-N biomarkers, that fulfilled at least one of the following criteria: (i) associations that were significant (at a Bonferroni threshold of p<9.09×10⁻⁴) in the full cohort; (ii) associations that were Bonferroni-significant in one of the two sexes; (iii) associations that showed suggestive significance (p<0.05) in one sex coupled with significance for effect heterogeneity between female and male effect estimates. Results for all metabolites, phenotypes and statistical models are provided in Table 6. Systematic comparison of estimated effects in men and women for all metabolites is shown in FIG. 2. Based on this comparison, we classified metabolite—A-T-N biomarker associations into homogenous effects if metabolites showed very similar effects in their association to the biomarker for both sexes and heterogeneous effects if effects showed major differences between the sexes with opposite effect directions of the same metabolite for men and women or substantially larger effects in one of the sexes. If an effect was heterogeneous and significant in males but not females or vice versa, we considered it sex-specific.

TABLE 5
Metabolite associations with A-T-N biomarkers that are either Bonferroni significant in the full sample, one sex,
or show nominal significance both in one sex and for effect heterogeneity. Given are regression results for the full sample
and both sexes, as well as heterogeneity estimates and the p-value for sex* metabolite interactions.

| | | | | | | |
|---|---|---|---|---|---|---|
| | | | | | | Inter- |
| Bio- | Met- | Ef- | Males | Females | Sex difference | action |

| mark- | ab- | | | p- | fect | | | p- | | | p- | | p- | | p- |
|---|---|---|---|---|---|---|---|---|---|---|---|---|---|---|---|
| er | olite | Effect | se | value | type | Effect | se | value | Effect | se | value | t | value | I² | value |
| Path- | PC ae | 0.283 | 0.078 | 2.58E | ho- | 0.282 | 0.102 | 5.96E | 0.299 | 0.121 | 1.33E | −0.1068 | 09.15E | 0.000 | 8.09E |
| olog- | C44:6 | | | −04 | mo- | | | −03 | | | −02 | | −01 | | −01 |
| ical | | | | | gene- | | | | | | | | | | |
| | | | | | ous | | | | | | | | | | |
| CSF | PC ae | 0.266 | 0.076 | 4.57E | ho- | 0.274 | 0.100 | 6.29E | 0.255 | 0.118 | 3.07E | 0.11904 | 9.05E | 0.000 | 7.83E |
| Aβ₁.₄₂ | C44:4 | | | −04 | mo- | | | −03 | | | −02 | | −01 | | −01 |
| | | | | | gene- | | | | | | | | | | |
| | | | | | ous | | | | | | | | | | |
| | PC ae | 0.260 | 0.075 | 5.23E | ho- | 0.294 | 0.100 | 3.26E | 0.214 | 0.116 | 6.38E | 0.52233 | 6.01E | 0.000 | 4.34E |
| | C44:5 | | | −04 | mo- | | | −03 | | | −02 | | −01 | | −01 |
| | | | | | gene | | | | | | | | | | |
| | | | | | ous- | | | | | | | | | | |
| | Thre- | 0.207 | 0.076 | 6.72E | male- | 0.372 | 0.112 | 8.83E | 0.070 | 0.108 | 5.17E | 1.943 | 5.20E | 48.545 | 4.03E |
| | onine | | | −03 | spe- | | | −04 | | | −01 | | −02 | | −02 |
| | | | | | cific | | | | | | | | | | |
| | Va- | −0.134 | 0.083 | 1.05E | het- | 0.032 | 0.114 | 7.80E | −0.299 | 0.123 | 1.50E | 1.973 | 4.85E | 49.322 | 7.65E |
| | line | | | −01 | ero- | | | −01 | | | −02 | | −02 | | −02 |
| | | | | | gene- | | | | | | | | | | |
| | | | | | ous | | | | | | | | | | |
| CSF | C10 | 0.084 | 0.030 | 4.58E | fe- | 0.014 | 0.042 | 7.34E | 0.144 | 0.042 | 6.07E | −2.203 | 2.76E | 54.613 | 2.55E |
| p-tau | | | | −03 | male- | | | −01 | | | −04 | | −02 | | −02 |
| | | | | | spe- | | | | | | | | | | |
| | | | | | cific | | | | | | | | | | |
| | C5- | 0.103 | 0.045 | 2.35E | het | 0.012 | 0.062 | 8.52E | 0.205 | 0.067 | 2.27E | −2.116 | 3.44E | 52.740 | 3.38E |
| | DC | | | −02 | ero- | | | −01 | | | −03 | | −02 | | −01 |
| | (C6- | | | | gene- | | | | | | | | | | |
| | OH) | | | | ous | | | | | | | | | | |
| | C8 | 0.064 | 0.030 | 3.42E | het | 0.003 | 0.041 | 9.39E | 0.127 | 0.045 | 5.11E | −2.028 | 4.26E | 50.692 | 5.63E |
| | | | | −02 | ero- | | | −01 | | | −03 | | −02 | | −02 |
| | | | | | gene- | | | | | | | | | | |
| | | | | | ous | | | | | | | | | | |
| | PC ae | 0.056 | 0.032 | 8.65E | het- | 0.129 | 0.046 | 4.80E | −0.023 | 0.046 | 6.18E | 2.355 | 1.85E | 57.535 | 2.16E |
| | C36:2 | | | −02 | ero- | | | −03 | | | −01 | | −02 | | −02 |
| | | | | | gene- | | | | | | | | | | |
| | | | | | ous | | | | | | | | | | |
| CSF | Histi- | −0.034 | 0.031 | 2.72E | het- | 0.033 | 0.042 | 4.39E | 0.045 | 0.045 | 1.97E | 2.237 | 2.53E | 55.290 | 2.42E |
| p-tau | dine | | | −01 | ero- | | | −01 | | | −02 | | −02 | | −02 |
| | | | | | gene- | | | | | | | | | | |
| | | | | | ous | | | | | | | | | | |
| | Aspar- | 0.034 | 0.031 | 2.84E | het- | 0.107 | 0.045 | 1.66E | −0.052 | 0.044 | 2.32E | 2.550 | 1.08E | 60.788 | 2.16E |
| | agine | | | −01 | ero- | | | −02 | | | −01 | | −02 | | −02 |
| | | | | | gene- | | | | | | | | | | |
| | | | | | ous | | | | | | | | | | |
| | SM | 0.032 | 0.031 | 3.10E | het- | 0.091 | 0.043 | 3.36E | −0.039 | 0.046 | 3.99E | 2.066 | 3.89E | 51.592 | 3.75E |
| | (OH) | | | −01 | ero- | | | −02 | | | −01 | | −02 | | −02 |
| | C16:1 | | | | gene- | | | | | | | | | | |
| | | | | | ous | | | | | | | | | | |
| | Gly- | 0.030 | 0.032 | 3.50E | het | 0.104 | 0.051 | 3.94E | −0.026 | 0.040 | 5.23E | 2.014 | 4.40E | 50.346 | 6.88E |
| | cine | | | −01 | ero- | | | −02 | | | −01 | | −02 | | −02 |
| | | | | | gene- | | | | | | | | | | |
| | | | | | ous | | | | | | | | | | |
| | PC ae | 0.028 | 0.031 | 3.68E | het | 0.088 | 0.043 | 4.17E | −0.041 | 0.044 | 3.51E | 2.094 | 3.62E | 52.251 | 3.76E |
| | C36:1 | | | −01 | ero- | | | −02 | | | −01 | | −02 | | −01 |
| | | | | | gene- | | | | | | | | | | |
| | | | | | ous | | | | | | | | | | |
| | C2 | 0.015 | 0.028 | 5.85E | het- | −0.054 | 0.039 | 1.67E | 0.089 | 0.041 | 3.02E | −2.527 | 1.15E | 60.430 | 1.39E |
| | | | | −01 | ero- | | | −01 | | | −02 | | −02 | | −02 |
| | | | | | gene- | | | | | | | | | | |
| | | | | | ous | | | | | | | | | | |
| FDG- | PC aa | −0.127 | 0.030 | 2.32E | ho- | −0.140 | 0.041 | 6.31E | −0.110 | 0.045 | 1.50E | −0.499 | 6.18E | 0.000 | 5.53E |
| PET | C32:1 | | | −05 | mo- | | | −04 | | | −02 | | −01 | | −01 |
| | | | | | gene- | | | | | | | | | | |
| | | | | | ous | | | | | | | | | | |
| | PC ae | −0.111 | 0.030 | 2.27E | ho- | −0.097 | 0.041 | 1.80E | −0.141 | 0.045 | 1.84E | 0.71633 | 4.74E | 0.000 | 2.21E |
| | C44:4 | | | −04 | mo- | | | −02 | | | −03 | | −01 | | −01 |
| | | | | | gene- | | | | | | | | | | |
| | | | | | ous | | | | | | | | | | |
| | PC ae | −0 .105 | 0.030 | 4.07E | ho- | −0.112 | 0.040 | 5.80E | −0.111 | 0.044 | 1.30E | −0.0207 | 9.83E | 0.000 | 6.02E |
| | C44:5 | | | −04 | mo- | | | −03 | | | −02 | | −01 | | −01 |
| | | | | | gene- | | | | | | | | | | |
| | | | | | ous | | | | | | | | | | |
| | PC aa | −0.107 | 0.032 | 6.85E | ho- | −0.125 | 0.045 | 5.67E | −0.091 | 0.045 | 4.25E | −0.547 | 5.84E | 0.000 | 7.44E |
| | C32:0 | | | −04 | mo- | | | −03 | | | −02 | | −01 | | −01 |
| | | | | | gene- | | | | | | | | | | |
| | | | | | ous | | | | | | | | | | |
| | PC ae | −0.103 | 0.031 | 8.56E | ho- | −0.103 | 0.042 | 1.58E | −0.112 | 0.045 | 1.33E | 0.15599 | 8.76E | 0.000 | 4.48E |
| | C42:4 | | | −04 | mo- | | | −02 | | | −02 | | −01 | | −01 |
| | | | | | gene- | | | | | | | | | | |
| | | | | | ous | | | | | | | | | | |
| | C16:1 | −0.103 | 0.031 | 9.09E | male- | −0.165 | 0.042 | 9.64E | −0.029 | 0.046 | 5.38E | −2.179 | 2.93E | 54.107 | 9.94E |
| | | | | −04 | spe- | | | −05 | | | −01 | | −02 | | −02 |
| | | | | | cific | | | | | | | | | | |
| | PC ae | −0.053 | 0.030 | 7.82E | het- | −0.119 | 0.042 | 4.34E | 0.016 | 0.044 | 7.15E | −2.238 | 2.52E | 55.312 | 5.78E |
| | C40:2 | | | −02 | ero- | | | −03 | | | −01 | | −02 | | −02 |
| | | | | | gene- | | | | | | | | | | |
| | | | | | ous | | | | | | | | | | |
| | Pro- | −0.023 | 0.031 | 4.51E | het- | 0.059 | 0.044 | 1.77E | −0.118 | 0.044 | 8.18E | 2.841 | 4.50E | 64.801 | 7.74E |
| | line | | | −01 | ero- | | | −01 | | | −03 | | −03 | | −03 |
| | | | | | gene- | | | | | | | | | | |
| | | | | | ous | | | | | | | | | | |

TABLE 6
Full association results for unstratified association analysis of metabolites with A-T-N biornarkers. as well as for 1-fold stratified
analysis by sex or APOE4 status having any p-value <0.05 (including heterogeneity estimates).

| | | | | | | | Men p- | Women | | Women p- | |
|---|---|---|---|---|---|---|---|---|---|---|---|
| Biomarker | Metabolite | Effect | se | p-value | Men Effect | Men se | value | effect | Women se | value | |
| Pathological | PC ae | 0.283 | 0.078 | 2.58E−04 | 0.282 | 0.102 | 5.96E−03 | 0.299 | 0.121 | 1.33E−02 | |
| CSF Aβ₁₋₄₂ | C44:6 | | | | | | | | | | |
| | PC ae | | | | | | | | | | |
| | 0444 | 0.265 | 0.076 | 4.57E−04 | 0.274 | 0.100 | 6.29E−03 | 0.255 | 0.118 | 3.07E−02 | |
| | PC ae | 0.260 | 0.075 | 5.23E−04 | 0.294 | 0.100 | 3.26E−03 | 0.214 | 0.116 | 6.38E−02 | |
| | C44:5 | | | | | | | | | | |
| | PC ae | 0.242 | 0.078 | 1.98E−03 | 0.265 | 0.104 | 1.04E−02 | 0.217 | 0.120 | 7.23E−02 | |
| | C424 | | | | | | | | | | |
| | Threonine | 0.207 | 0.076 | 6.72E−03 | 0.372 | 0.112 | 8.83E−04 | 0.070 | 0.108 | 5.17E−01 | |
| | Valine | −0.134 | 0.083 | 1.05E−01 | 0.032 | 0.114 | 7.80E−01 | −0.299 | 0.123 | 1.50E−02 | |
| | Proline | −0.075 | 0.081 | 3.52E−01 | −0.002 | 0.114 | 9.87E−01 | −0.163 | 0.118 | 1.67E−01 | |
| | Gycine | 0.060 | 0.082 | 4.60E−01 | 0.244 | 0.131 | 6.19E−02 | −0.079 | 0.108 | 4.63E−01 | |
| | C8 | 0.015 | 0.080 | 8.54E−01 | 0.035 | 0.106 | 7.42E−01 | −0.022 | 0.123 | 8.59E−01 | |
| CSF p-tau | C10 | 0.084 | 0.030 | 4.58E−03 | 0.014 | 0.042 | 7.34E−01 | 0.144 | 0.042 | 6.07E−04 | |
| | C5-DC | 0.103 | 0.045 | 2.35E−02 | 0.012 | 0.062 | 8.52E−01 | 0/205 | 0.067 | 2.27E−03 | |
| | (C6-OH) | | | | | | | | | | |
| | C8 | 0.064 | 0.030 | 3.42E−02 | 0.003 | 0.041 | 9.39E−01 | 0.127 | 0.045 | 5.11E−03 | |
| | PC ae | 0.056 | 0.032 | 8.65E−02 | 0.129 | 0.046 | 4.80E−03 | −0.023 | 0.046 | 6.18E−01 | |
| | C36:2 | | | | | | | | | | |
| | Histidine | −0.034 | 0.031 | 2.72E−01 | 0.033 | 0.042 | 4.39E−01 | −0.105 | 0.045 | 1.97E−02 | |
| | Asparagine | 0.034 | 0.031 | 2.84E−01 | 0.107 | 0.045 | 1.66E−02 | −0.052 | 0.044 | 2.32E−01 | |
| | SM (OH) | 0.032 | 0.031 | 3.10E−01 | 0.091 | 0.043 | 3.36E−02 | −0.039 | 0.046 | 3.99E−01 | |
| | C16:1 | | | | | | | | | | |
| | Glycine | 0.030 | 0.032 | 3.50E−01 | 0.104 | 0.051 | 3.94E−02 | −0.026 | 0.040 | 5.23E−01 | |
| | PC ae | 0.028 | 0.031 | 3.68E−01 | 0.088 | 0.043 | 4.17E−02 | −0.041 | 0.044 | 3.51E−01 | |
| | C36:1 | | | | | | | | | | |
| | C2 | 0.015 | 0.028 | 5.85E−01 | −0.054 | 0.039 | 1.67E−01 | 0.089 | 0.041 | 3.02E−02 | |
| FDG ET | PC aa | −0.127 | 0.030 | 2.32E−05 | −0.140 | 0.041 | 6.31E−04 | −0.110 | 0.045 | 1.50E−02 | |
| | C321 | | | | | | | | | | |
| | PC a | −0.111 | 0.030 | 2.27E−04 | −0.097 | 0.041 | 1.80E−02 | −0.141 | 0.045 | 1.84E−03 | |
| | C444 | | | | | | | | | | |
| | PC ae | −0.105 | 0.030 | 4.07E−04 | −0.112 | 0.040 | 5.80E−03 | −0.111 | 0.044 | 1.30E−02 | |
| | C44:5 | | | | | | | | | | |
| | PC aa | −0.107 | 0.032 | 6.85E−04 | −0.125 | 0.045 | 5.67E−03 | −0.091 | 0.045 | 4.25E−02 | |
| | C320 | | | | | | | | | | |
| | PC ae | −0.103 | 0.031 | 8.56E−04 | −0.103 | 0.042 | 1.58E−02 | −0.112 | 0.045 | 1.33E−02 | |
| | C424 | | | | | | | | | | |
| | C16:1 | −0.103 | 0.031 | 9.09E−04 | −0.165 | 0.042 | 9.64E−05 | −0.029 | 0.046 | 5.38E−01 | |
| | C10 | −0.057 | 0.029 | 5.14E−02 | −0.076 | 0.041 | 6.15E−02 | −0.032 | 0.043 | 4.60E−01 | |
| | PC ae | −0.053 | 0.030 | 7.82E−02 | −0.119 | 0.042 | 4.34E−03 | 0.016 | 0.044 | 7.15E−01 | |
| | C40:2 | | | | | | | | | | |
| | C8 | −0.051 | 0.031 | 9.96E−02 | −0.075 | 0.041 | 6.87E−02 | −0.010 | 0.047 | 8.34E−01 | |
| | Valine | 0.036 | 0.032 | 2.49E−01 | 0.021 | 0.044 | 6.40E−01 | 0.052 | 0.046 | 2.50E−01 | |
| | Glycine | −0.032 | 0.031 | 3.00E−01 | −0.060 | 0.050 | 2.34E−01 | -0.018 | 0.040 | 6.48E−01 | |
| | Prone | −0.023 | 0.031 | 4.51E−01 | 0.059 | 0.044 | 1.77E−01 | -0.118 | 0.044 | 8.18E−03 | |
| | Sex cliff | | AOPOE4+ | AOPOE | AOPOE4+ | AOPOE | AOPOE4− | AOPOE4− | AOPOE | AOPOE | AOPOE |
| Sex cliff t | p-value | Sex diff I² | Effect | 4+ Sc | p-value | 4− Effect | se | p-value | diff t | diff p | diff I² |
| -0.107 | 9.15E−01 | 0.000 | 0.630 | 0.150 | 2.50E−05 | 0.158 | 0.090 | 7.96E−02 | −2.705 | 6.83E−03 | 63.030 |
| 0.119 | 9.05E−01 | 0.000 | 0.565 | 0.148 | 1.30E−04 | 0.139 | 0.088 | 1.13E−01 | −2.478 | 1.32E−02 | 59.645 |
| 0.522 | 6.01E−01 | 0.000 | 0.609 | 0.145 | 2.64E−05 | 0.129 | 0.087 | 1.37E−01 | −2.837 | 4.56E−03 | 64.749 |
| 0.308 | 7.58E−01 | 0.000 | 0.564 | 0.148 | 1.32E−04 | 0.114 | 0.092 | 2.15E−01 | −2.589 | 9.61E−03 | 61.382 |
| 1.943 | 5.20E−02 | 48.545 | 0.347 | 0.139 | 1.26E−02 | 0.141 | 0.092 | 1.25E−01 | −1.235 | 2.17E−01 | 19.027 |
| 1.973 | 4.85E−02 | 49.322 | −0.201 | 0.141 | 1.53E−01 | −0.142 | 0.101 | 1.60E−01 | 0.345 | 7.30E−01 | 0.000 |
| 0.986 | 3.24E−01 | 0.000 | 0.176 | 0.142 | 2.15E−01 | −0.202 | 0.100 | 4.40E−02 | −2.173 | 2.98E−02 | 53.982 |
| 1.908 | 5.64E−02 | 47.582 | 0.363 | 0.154 | 1.83E−02 | −0.102 | 0.100 | 3.05E−01 | −2.538 | 1.11E−02 | 60.604 |
| 0.349 | 7.27E−01 | 0.000 | −0.251 | 0.136 | 6.39E−02 | 0.120 | 0.097 | 2.15E−01 | 2.229 | 2.58E−02 | 55.134 |
| −2.203 | 2.76E−02 | 54.613 | 0.102 | 0.047 | 3.07E−02 | 0.070 | 0.042 | 9.71E−02 | −0.501 | 6.16E−01 | 0.000 |
| −2.116 | 3.44E−02 | 52.740 | 0.086 | 0.071 | 2.29E−01 | 0.106 | 0.066 | 1.07E−01 | 0.205 | 8.37E−01 | 0.000 |
| −2.028 | 4.26E−02 | 50.692 | 0.064 | 0.046 | 1.68E−01 | 0.060 | 0.045 | 1.83E−01 | −0.058 | 9.54E−01 | 0.000 |
| 2.355 | 1.85E−02 | 57.535 | 0.005 | 0.051 | 9.19E−01 | 0.107 | 0.047 | 2.30E−02 | 1.479 | 1.39E−01 | 32.408 |
| 2.237 | 2.53E−02 | 55.290 | −0.046 | 0.049 | 3.45E−01 | −0.032 | 0.044 | 4.65E−01 | 0.210 | 8.33E−01 | 0.000 |
| 2.550 | 1.08E−02 | 60.788 | 0.002 | 0.050 | 9.70E−01 | 0.044 | 0.045 | 3.21E−01 | 0.632 | 5.27E−01 | 0.000 |
| 2.066 | 3.89E−02 | 51.592 | −0.019 | 0.050 | 7.08E−01 | 0.082 | 0.045 | 6.90E−02 | 1.500 | 1.34E−01 | 33.353 |
| 2.014 | 4.40E−02 | 50.346 | 0.075 | 0.050 | 1.30E−01 | −0.003 | 0.046 | 9.41E−01 | −1.160 | 2.46E−01 | 13.825 |
| 2.094 | 3.62E−02 | 52.251 | −0.017 | 0.049 | 7.21E−01 | 0.060 | 0.045 | 1.81E−01 | 1.169 | 2.42E−01 | 14.441 |
| −2.527 | 1.15E−02 | 60.430 | 0.003 | 0.043 | 9.40E−01 | 0.020 | 0.042 | 6.35E−01 | 0.278 | 7.81E−01 | 0.000 |
| −0.499 | 6.18E−01 | 0.000 | −0.087 | 0.045 | 5.35E−02 | −0.162 | 0.042 | 1.34E−04 | −1.210 | 2.26E−01 | 17.332 |
| 0.716 | 4.74E−01 | 0.000 | −0.115 | 0.047 | 1.39E−02 | −0.114 | 0.041 | 6.34E−03 | 0.023 | 9.82E−01 | 0.000 |
| −0.021 | 9.83E−01 | 0.000 | −0.122 | 0.046 | 8.34E−03 | −0.102 | 0.041 | 1.30E−02 | 0.326 | 7.44E−01 | 0.000 |
| −0.547 | 5.84E−01 | 0.000 | −0.135 | 0.047 | 4.39E−03 | −0.082 | 0.045 | 6.58E−02 | 0.818 | 4.14E−01 | 0.000 |
| 0.156 | 8.76E−01 | 0.000 | −0.131 | 0.047 | 5.79E−03 | −0.066 | 0.043 | 4.51E−02 | 0.701 | 4.33E−01 | 0.000 |
| −2.179 | 2.93E−02 | 54.107 | −0.084 | 0.048 | 7.61E−02 | −0.120 | 0.043 | 5.15E−03 | −0.557 | 5.78E−01 | 0.000 |
| −0.750 | 4.54E−01 | 0.000 | 0.037 | 0.046 | 4.17E−01 | −0.135 | 0.040 | 7.17E−04 | −2.840 | 4.51E−03 | 64.793 |
| −2.238 | 2.52E−02 | 55.312 | −0.078 | 0.044 | 7.94E−02 | −0.035 | 0.043 | 4.19E−01 | 0.696 | 4.86E−01 | 0.000 |
| −1.040 | 2.98E−01 | 3.830 | 0.038 | 0.046 | 4.04E−01 | −0.138 | 0.043 | 1.58E−03 | −2.794 | 5.20E−03 | 64.215 |
| −0.498 | 6.19E−01 | 0.000 | −0.040 | 0.048 | 4.08E−01 | 0.106 | 0.044 | 1.68E−02 | 2.234 | 2.55E−02 | 55.233 |
| −0.647 | 5.18E−01 | 0.000 | −0.140 | 0.047 | 3.05E−03 | 0.059 | 0.044 | 1.80E−01 | 3.092 | 1.99E−03 | 67.653 |
| 2.841 | 4.50E−03 | 64.801 | −0.100 | 0.047 | 3.39E−02 | 0.048 | 0.043 | 2.64E−01 | 2.324 | 2.01E−02 | 56.977 |

TABLE 7
Associations of metabolites identified in the sex-centric analysis with A-T-N biomarkers that are either Bonferroni significant in the full
sample, in APOE4+ or APOE4− subjects, or show nominal significance both in one APOE4 status group and for effect heterogeneity. Given are
regression results for the full sample and both APOE4 status groups. as well as heterogeneity estimates and the p-value for APOE4 status*
metabolite interactions.

| | | | | | | | | | | | | | | | Interac- |
|---|---|---|---|---|---|---|---|---|---|---|---|---|---|---|---|
| Bio- | Metab- | | | p- | Effect | APOE44⁺ | | | APOE4⁻⁻ | | | APOE4 status difference | | | tion |
| marker | olite | Effect | se | value | type | Effect | se | p-value | Effect | se | p-value | t | p-value | I² | p-value |
| Patho- | PC ae | 0.283 | 0.078 | 2.58E | specific | 0.630 | 0.150 | 2.50E | 0.158 | 0.090 | 7.96E | −2.705 | 6.83E | 63.030 | 2.80E |
| logical | C44:6 | | | −04 | to ε4+ | | | −05 | | | −02 | | −03 | | −03 |
| CSF | | | | | | | | | | | | | | | |
| Aβ1-42 | PC ae | 0.265 | 0.076 | 4.57E | specific | 0.565 | 0.148 | 1.30E | 0.139 | 0.088 | 1.13E | −2.478 | 1.32E | 59.645 | 5.80E |
| | C44:4 | | | −04 | to ε4+ | | | −04 | | | −01 | | −02 | | −03 |
| | PC ae | 0.260 | 0.075 | 5.23E | specific | 0.609 | 0.145 | 2.64E | 0.129 | 0.087 | 1.37E | −2.837 | 4.56E | 64.749 | 3.07E |
| | C44:5 | | | −04 | to ε4+ | | | −05 | | | −01 | | −03 | | −03 |
| | PC ae | 0.242 | 0.078 | 1.98E | specific | 0.564 | 0.148 | 1.32E | 0.114 | 0.092 | 2.15E | −2.589 | 9.61E | 61.382 | 5.64E |
| | C42:4 | | | −03 | to ε4+ | | | −04 | | | −01 | | −03 | | −03 |
| | Proline | −0.075 | 0.081 | 3.52E | hetero- | 0.176 | 0.142 | 2.15E | −0.202 | 0.100 | 4.40E | −2.173 | 2.98E | 53.982 | 1.58E |
| | | | | −01 | geneous | | | −01 | | | −02 | | −02 | | −01 |
| | Glycine | 0.060 | 0.082 | 4.60E | hetero- | 0.363 | 0.154 | 1.83E | −0.102 | 0.100 | 3.05E | −2.538 | 1.11E | 60.604 | 7.89E |
| | | | | −01 | geneous | | | −02 | | | −01 | | −02 | | −04 |
| FDC- | PC aa | −0.127 | 0.030 | 2.32E | homo- | −0.087 | 0.045 | 5.35E | −0.162 | 0.042 | 1.34E | −1.210 | 2.26E | 17.332 | 3.58E |
| PET | C32:1 | | | −05 | geneous | | | −02 | | | −04 | | −01 | | −01 |
| | PC ae | −0.111 | 0.030 | 2.27E | homo- | −0.115 | 0.047 | 1.39E | −0.114 | 0.041 | 6.34E | 0.023 | 9.82E | 0.000 | 8.63E |
| | C44:4 | | | −04 | geneous | | | −02 | | | −03 | | −01 | | −01 |
| | PC ae | −0.105 | 0.030 | 4.07E | homo- | −0.122 | 0.046 | 8.34E | −0.102 | 0.041 | 1.30E | 0.326 | 7.44E | 0.000 | 6.39E |
| | C44:5 | | | −04 | geneous | | | −03 | | | −02 | | −01 | | −01 |
| | PC aa | −0.107 | 0.032 | 6.85E | homo | −0.135 | 0.047 | 4.39E | −0.082 | 0.045 | 6.58E | 0.818 | 4.14E | 0.000 | 3.69E |
| | C32:0 | | | −04 | geneous | | | −03 | | | −02 | | −01 | | −01 |
| | PC ae | −0.103 | 0.031 | 8.56E | homo- | −0.131 | 0.047 | 5.79E | −0.086 | 0.043 | 4.51E | 0.701 | 4.83E | 0.000 | 3.98E |
| | C42:4 | | | −04 | geneous | | | −03 | | | −02 | | −01 | | −01 |
| | C10 | −0.057 | 0.029 | 5.14E | specific | 0.037 | 0.046 | 4.17E | −0.135 | 0.040 | 7.17E | −2.840 | 4.51E | 64.793 | 4.96E |
| | | | | −02 | to ε4+ | | | −01 | | | −04 | | −03 | | −03 |
| | C8 | −0.051 | 0.031 | 9.96E | hetero- | 0.038 | 0.046 | 4.04E | −0.138 | 0.044 | 1.58E | −2.794 | 5.20E | 64.215 | 6.37E |
| | | | | −02 | geneous | | | −01 | | | −03 | | −03 | | −03 |
| | Valine | 0.036 | 0.032 | 2.49E | hetero- | −0.040 | 0.048 | 4.08E | 0.106 | 0.044 | 1.68E | 2.234 | 2.55E | 55.233 | 9.50E |
| | | | | −01 | geneous | | | −01 | | | −02 | | −02 | | −02 |
| | Glycine | −0.032 | 0.031 | 3.00E | hetero- | −0.140 | 0.047 | 3.05E | 0.059 | 0.044 | 1.80E | 3.092 | 1.99E | 67.653 | 3.29E |
| | | | | −01 | geneous | | | −03 | | | −01 | | −03 | | −03 |
| | Proline | −0.023 | 0.031 | 4.51E | hetero- | −0.100 | 0.047 | 3.39E | 0.048 | 0.043 | 2.64E | 2.324 | 2.01E | 56.977 | 6.35E |
| | | | | −01 | geneous | | | −02 | | | −01 | | −02 | | −02 |

TABLE 8
Significant metabolite effects in the combined stratification (sex by APOE4 status) on A-T-N biomarkers are driven by or limited to
APOE4+ females. Given are regression results for the full sample. APOE4+ males. APOE4+ females. as well as heterogeneity estimates by
sex and APOE4 status. The only metabolite showing effect heterogeneity for both stratification variables was proline in its association with
FDG-PET values. Sex cliff.: sex-difference. APOE4 cliff.: difference between APOE4-1+ status groups.

| | | | | | | APOE4 | | APOE4+ | APOE4+ | APOE4+ | APOE4+ |
|---|---|---|---|---|---|---|---|---|---|---|---|
| | | | | Sex diff. | Sex diff. | diff. p- | APOE4 | Males | Males p- | Females | Females |
| Biomarker | Metabolite | Effect | p-value | p-value | I² | value | diff. I² | effect | value | effect | p-value |
| Pathological | PC ae | 0.283 | 2.58E−04 | 9.15E−01 | 0.000 | 6.83E−03 | 63.030 | 0.463 | 1.68E−02 | 0.922 | 1.90E−04 |
| | C44:6 | | | | | | | | | | |
| CSF Aβ1-42 | PC ae | 0.260 | 5.23E−04 | 6.01E−01 | 0.000 | 4.56E−03 | 64.749 | 0.521 | 6.17E−03 | 0.761 | 8.29E−04 |
| | C44:5 | | | | | | | | | | |
| | PC ae | 0.242 | 1.98E−03 | 7.58E−01 | 0.000 | 9.61E−03 | 61.382 | 0.420 | 3.15E−02 | 0.761 | 8.65E−04 |
| | C42:4 | | | | | | | | | | |
| CSF p-tau | C10 | 0.084 | 4.58E−03 | 2.76E−02 | 54.613 | 6.16E−01 | 0.000 | −0.064 | 3.24E−01 | 0.264 | 1.21E−04 |
| FDG-PET | Proline | −0.023 | 4.51E−01 | 4.50E−03 | 64.801 | 2.01E−02 | 56.977 | 0.046 | 4.76E−01 | −0.272 | 8.22E−05 |

### Homogeneous Effects

We refer to homogenous effects where similar alterations in metabolite levels are associated with AD biomarkers in men and women. Metabolites with homogenous effects lie on or close to the diagonal going through the first and third quadrant when plotting the effect estimates in women against those in men in FIG. 2. We identified eight significant homogenous metabolite-phenotype associations with A-T-N biomarkers: CSF Aβ₁₋₄₂ pathology was significantly associated with levels of three related acyl-alkyl-PCs (PC ae C44:4, PC ae C44:5, PC ae C44:6). Two of those (PC ae C44:4 and PC ae C44:5) were also significantly associated with brain glucose uptake (FDG-PET) in addition to three other PCs (PC aa C32:1, PC aa C32:0 and PC ae C42:4). For p-tau, we did not identify any homogeneous, overall significant associations. Notably, none of the associations categorized as homogenous showed any indication of effect heterogeneity between sexes, and only one association reached significance in the sex-stratified analyses: higher blood levels of the diacyl-PC PC aa C32:1 were associated with lower glucose uptake in brain in the male stratum alone despite lower power.

### Heterogeneous Effects

We refer to heterogeneous effects where a metabolite shows opposite effect directions for the same phenotype in men and women, or substantially larger effects in one sex leading to significant heterogeneity and/or sex-metabolite interaction. Metabolites showing these types of effects fall mainly into the second or fourth quadrant (with the exception of sex-specific effects) when contrasting the effect estimates for men and women in the plots for the three A-T-N phenotypes in FIG. 2. In our study, we identified a total of 15 associations in this category (including three sex-specific effects). For CSF Aβ₁₋₄₂, we identified two heterogeneous effects with threonine showing a sex-specific effect (see paragraph below) with greater effect size in males and valine with a larger effect in females: while valine was not significantly associated (P=0.78) with CSF Aβ₁₋₄₂ pathology in males, in females, it showed a nominally significant negative association with an estimated heterogeneity of I²=49.3%. CSF p-tau was the biomarker with the largest number of heterogeneous associations: acylcarnitines C5-DC (C6-OH), C8, C10 (sex-specific), and C2, as well as the amino acid histidine showed stronger associations in females, while the related acyl-alkyl-PCs PC ae C36:1 and PC ae C36:2, the amino acids asparagine and glycine, and one hydroxy-SM (SM (OH) C16:1) yielded stronger associations in males (all I²>50%); associations with FDG-PET revealed three heterogeneous effects, with acyl-alkyl PC ae C40:2, and the acylcarnitine C16:1 (sex-specific) showing a larger effect in males (I²=55.3%), and proline having a larger effect in females (I²=64.8%). Notable, 9 of the 15 reported heterogeneous associations showed opposite effect directions between sexes, and in 7 cases, the interaction term (sex*metabolite) was also significantly (at p<0.05) associated with the respective biomarker.

### Sex-Specific Effects

We refer to sex-specific effects where metabolite associations are only significant in one sex with either significant effect heterogeneity between males and females or significant sex-metabolite interaction. In FIG. 2, metabolites with this effect category fall into the area close to the x- (male-specific) or y- (female-specific) axes of the three effect plots for the different A-T-N phenotypes. In total, we found three instances of this effect type. Male-specific effects were seen for threonine with pathological CSF Aβ₁₋₄₂ (positive association) and C16:1 with FDG-PET (negative association). We also identified a single female-specific effect, where higher levels of the medium-chain acylcarnitine C10 were associated with higher CSF p-tau. This association was simultaneously the strongest seen for p-tau in the analysis of the full cohort yet seems to be driven by female effects only.

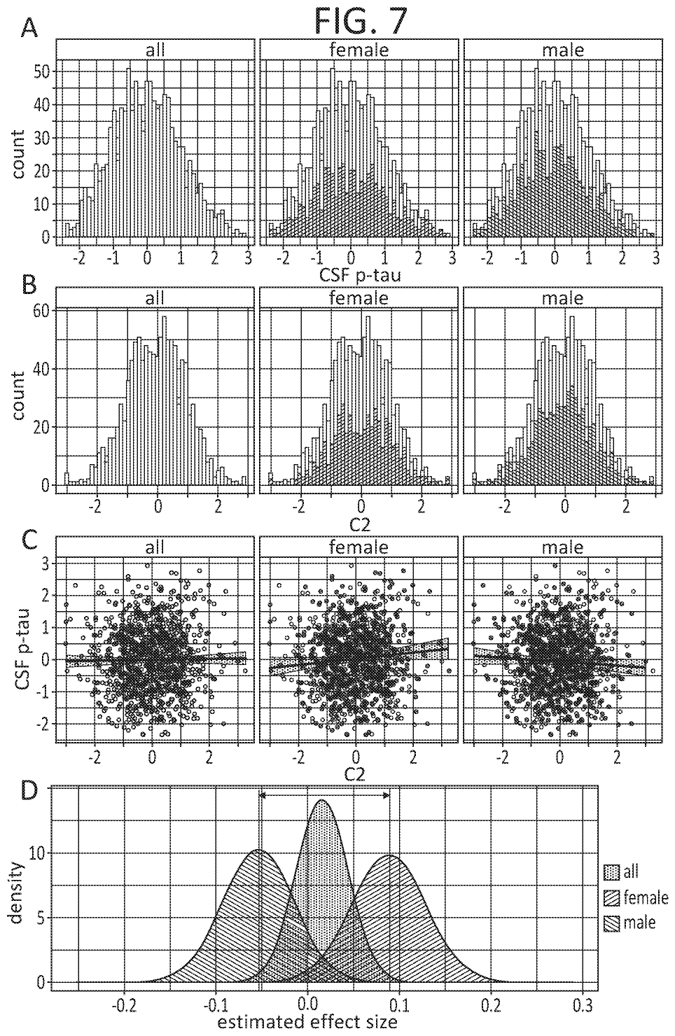

We assessed the significance of the effect of heterogeneity as exemplified by the association of C2 with CSF p-tau (FIG. 7A-D). Heterogeneity estimation as applied here investigates if there is a significant difference in the estimated distributions of association effects seen for two strata (here, females and males). These analyses are not directly aiming at determining the direction of effects in one of the strata (which may change for insignificant associations), but are examining if there is a significant difference in the variance-weighted estimated effects. A significant test thus identifies associations that show significantly different effects in two strata that cannot be identified in a pooled analysis adjusting for the stratifying variable, as seen in the density curve of the estimated effect distribution across all samples (which is centered close to 0). These analyses are naturally assuming a close-to-normal distribution of effect sizes (as is regression), which we could validate for >90% of investigated effects.

### Intertwined Modulation of Metabolite Effects by Sex and APOE

Previous reports suggested that the APOE ε4 genotype may exert AD risk predisposition in a sex-dependent way [8-13]. In order to investigate potential relationships between sex and APOE4 status on the metabolomic level, we selected the 21 metabolites identified in the previous analyses (Table 5) and performed association analyses with the three selected A-T-N biomarkers, now stratified by APOE4 status and adjusted for sex. Using the same effect categories (homogeneous, heterogeneous, and group-specific) as for the sex-stratified analyses revealed that metabolite effects in APOE ε4 carriers vs. non-carriers also show effects from all three categories (Table 7): homogeneous effects were noted for the overall significant associations of PC aa C32:1, PC ae C44:4, PC ae C44:5, PC aa C32:0, and PC ae C42:4 with FDG-PET. Heterogeneous effects again formed the largest group (n=11), with proline and glycine showing opposite effect directions on CSF Aβ₁₋₄₂ pathology and C8, valine, glycine, and proline having opposite effect directions on FDG-PET for E4 carriers vs. non-carriers, respectively. 5 metabolites with heterogeneous effects even showed APOE4 status-specific effects: (i) the associations of PC ae C44:6, PC ae C44:4, PC ae C44:5, and PC ae C42:4 with pathological CSF Aβ₁₋₄₂ in APOE ε4 carriers. In case of PC ae C44:6, PC ae C44:5, and PC ae C44:4, the group-specific effects were strong enough to drive the signal to overall significance in the full sample. (ii) the association of acylcarnitine C10 with FDG-PET in APOE ε4 non-carriers.

### Some Metabolic Effects are Specific to Female e4 Carriers.

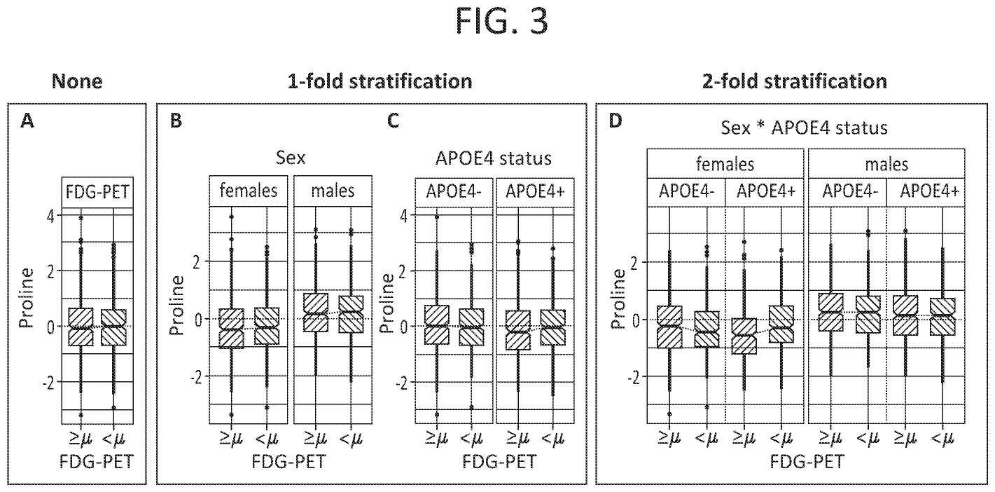

When we stratified separately by sex and APOE4 status, we observed several metabolites (C8, C10, valine, glycine, and proline) that showed heterogeneous effects on AD biomarkers in both stratifications. To investigate potential additional subgroup-specific effects, we combined the two stratifications and investigated the selected metabolite set for sex-by-APOE4 status effect modulations. Although the group of APOE ε4-carrying women was the smallest among the four strata, all Bonferroni-significant associations were found in this subgroup (Table 8): higher levels of three acyl-alkyl PCs (PC ae C42:4, PC ae C44:5, and PC ae C44:6) were associated with pathological CSF Aβ₁₋₄₂, higher acylcarnitine C10 was associated with increased CSF p-tau, and higher proline levels were associated with decreased FDG-PET values (FIG. 3). The latter association was not observed in any other of the performed analyses. Of note, except for the association of C10 with p-tau, we found significant (p<0.05) interaction effects between the metabolites and APOE4 status on their associated endophenotypes in females only, while the effects in males were not significantly (p>0.1) modulated by APOE4 status.

### Estimates of Effects and Effect Heterogeneity are Stable.

To investigate the robustness of findings reported in this study, we performed 1000 bootstrap re-samplings for each A-T-N biomarker to generate simulated population-based effect distributions for all significant associations. Overall, the difference between effect estimates obtained in the three rounds of original analyses (pooled sample, onefold, and twofold stratification) and the respective average effect estimate across all bootstraps (i.e., the variability by means of estimated bias) was marginal. We also did not find any instance of an originally significant association (at PREG≤0.05) where the bootstrap-t 95% confidence interval contained zero. This means that the simulated population effect as estimated by bootstrapping is unequal to zero, suggesting robustness of our reported findings. Further, 91.97% of simulated effect distributions were normally distributed (PShapiro-Wilk>0.05). Bootstrapping replicated significance of associations at the respective p value thresholds and the expected (post hoc) power of ≥50% with only three exceptions: estimated effect heterogeneity between sexes for the association of valine with pathological CSF Aβ₁₋₂ was, although on average (i.e., averaged across all 1000 samples) significant, only significant in 49.9% of bootstraps; the significant associations of PC ae C44:5 and PC ae C42:4 with pathological CSF Aβ₁₋₄₂ in APOE ε4-carrying females on average narrowly missed the Bonferroni-corrected significance threshold (PREG=9.45×10⁻⁴ and PREG=9.77×10⁻⁴, respectively), although both metabolites showed Bonferroni-significant p values in >50% of bootstraps.

### Replication of Results in Independent Cohorts

To the best of our knowledge, ADNI is currently the only study of AD with data on both AD biomarkers and metabolite levels with sufficient sample sizes to conduct the reported analyses. Estimates of required sample sizes for replication of our findings are provided in Table 2. We nevertheless sought independent replication of our results in two other studies with subsets of the examined variables available: (i) the Rush Religious Order Study and the Rush Memory and Aging Project (ROS/MAP), for which we had access to 126 and 137 data points with data on p180 metabolites and data on overall amyloid load and severity of tau pathology in the brain (based on post-mortem neuropathology assessment), respectively (Supplementary Note 1). (ii) the Australian Imaging, Biomarker & Lifestyle Flagship Study of Ageing (AIBL) with data on CSF p-tau and comparable measurements of three lipid species (PC ae C36:1, PC ae C36:2, and SM (OH) C16:1) in 94 participants. Both studies had less than one quarter of the mean required sample size (n=677).

We were able to replicate all homogeneous associations reported for pathological CSF Aβ1-42 (PC ae C44:6, PC ae C44:5, and PC ae C44:4) in ROS/MAP at p values significant after Bonferroni correction (PREG<2.94×10⁻³) and with the same effect directions as in ADNI, despite the different measure for Aβ pathology. For eight of the 14 sex- and APOE ε4 status-stratified associations for Aβ and tau pathology, we observed non-zero effect heterogeneity estimates (I2 of 1.4-45.7%), albeit non-significant. The three metabolite measures in AIBL all showed non-zero effect heterogeneity estimates (I2 of 39.7-54.3%) in the sex-stratified analyses with CSF p-tau, with effect heterogeneity being significant for SM (OH) C16:1 (PHET=0.016). Combined, AIBL and ROS/MAP yielded non-zero heterogeneity estimates for two out of four reported group comparisons for Aβ pathology and eight out of ten reported group comparisons for CSF p-tau and brain tau pathology.

In this study, we investigated the influence of sex and APOE4 status on metabolic alterations related to representative A-T-N biomarkers (CSF Aβ₁₋₄₂ pathology (A), CSF p-tau (T), FDG-PET (N)). By stratified analyses and systematic comparison of the effects estimated for the two sexes, we revealed substantial differences between men and women in their associations of blood metabolite levels with these AD biomarkers, although known sexual dimorphisms of metabolite levels themselves were unaffected by the disease.

Differences between the sexes were largest for associations of metabolites and CSF p-tau levels. Notably, this biomarker was not significantly associated with any metabolite when including all subjects and adjusting for both sex and copies of APOE ε4, yet association analysis stratified by sex (but still adjusted for copies of APOE ε4) revealed a significant, female-specific metabolite/CSF p-tau association despite the smaller sample size. In contrast, for CSF Aβ₁₋₄₂ and FDG-PET, in addition to heterogeneous, sex-specific effects, we also found homogenous effects, where metabolite concentrations showed the same trends of metabolite levels correlating with CSF Aβ₁₋₄₂ pathology and/or lower brain glucose uptake in both sexes.

For many of the metabolites with different effects for the sexes, we additionally observed significant effect heterogeneity between carriers and non-carriers of the APOE ε4 allele, suggesting intertwined modulation of metabolic effects by sex and APOE genotype. Indeed, two-fold stratification revealed metabolite associations that were either driven by or even specific to the group with presumably highest risk, APOE ε4 carrying females. Our results, thus, demonstrate the importance of stratified analyses for getting insights into metabolic underpinnings of AD that are seemingly restricted to a specific group of patients.

The metabolites showing effect heterogeneity across AD biomarkers in this study highlight sex-specific dysregulations of energy metabolism (acylcarnitines C2, C5-DC/C6-OH, C8, C10 and C16:1 for lipid-based energy metabolism [64]; amino acids valine, glycine, and proline as markers for glucogenic and ketogenic energy metabolism [65-67]), energy homeostasis (asparagine, glycine, proline, and histidine [66-70]), and (metabolic/nutrient) stress response (threonine, proline, histidine [67, 69, 71]). While these pathways have been linked to AD before, our work presents first evidence and molecular readouts for sex-related metabolic differences in AD.

For instance, in our previous report, we discussed the implication of failing lipid energy metabolism in the context of AD biomarker profiles, starting at the stage of pathological changes in CSF tau levels [44]. The current study now provides further insights in this topic, marking this finding to be predominant in females. More specifically, we observed a significant female-specific association of higher levels of acylcarnitine C10 with increased levels of CSF p-tau, with two other metabolites of this pathway (C8 and C5-DC/C6-OH) narrowly falling short of meeting the Bonferroni threshold. This indicates a sex-specific buildup of medium-chain fatty acids in females, suggesting increased energy demands coupled with impaired energy production via mitochondrial beta-oxidation [64].

Interestingly, the significant heterogeneity of association results between sexes for CSF p-tau and glycine, with higher levels of glycine being linked to higher levels of CSF p-tau in men, indicates that energy demands are equally upregulated in males as in females. In contrast to women, men, however, appear to compensate this demand by upregulation of glucose energy metabolism as glycine is a positive marker of active glucose metabolism and insulin sensitivity [66]. Findings for acylcarnitines in females are further contrasted by the observed male-specific association of higher levels of the long-chain acylcarnitine C16:1 with decreased brain glucose uptake, which might indicate that in males there is a switch to provision of fatty acids as alternative fuel when glucose-based energy metabolism is less effective. As we did not observe the buildup of medium- and short-chain acylcarnitines as seen in females, we assume that, in males, energy production via mitochondrial beta-oxidation may be sustained, at least in early disease.

Evidence corroborating sex-specific processes in energy homeostasis linked to changes in CSF p-tau levels is provided by the significant heterogeneity estimates for histidine with lower levels of histidine being linked to higher levels of CSF p-tau in women. Depletion of histidine has been shown to be associated with insulin resistance, inflammatory processes, as well as oxidative stress, especially in women with metabolic dysregulation [68, 69].

We further identified a heterogeneous association of valine with lower levels in females (P<0.05), but not in males, with Aβ₁₋₄₂ pathology. Valine, a BCAA and important energy carrying molecule, has been reported to be associated with cognitive decline and brain atrophy in AD, as well as with risk for incident dementia [41, 44]. The lower levels observed in AD are in contrast to other complex phenotypes such as type 2 diabetes, insulin resistance, or obesity [65, 72], where higher levels of BCAAs are found, and may indicate a switch to increased energy consumption via degradation of amino acids in AD. A recent study highlighted decreasing levels of valine as being significantly associated with all-cause mortality [73]. Besides implications for energy metabolism, results from our study may thus characterize lower levels of valine also as a marker for increased female vulnerability to pathogenic processes in general and to P-amyloidosis in AD in particular.

The higher effect size of genetic risk for AD exerted by the APOE ε4 allele in females compared to males still awaits molecular elucidation. Here, we tried to elaborate on potential interrelated risk predispositions from a metabolomic point of view. We therefore investigated if APOE4 status may also modulate metabolic readouts of AD-linked A-T-N biomarker profiles identified in sex-centered analyses. We found that indeed the majority (68.8%) of observed associations between metabolites and AD biomarkers shows significant heterogeneity between APOE4 status groups.

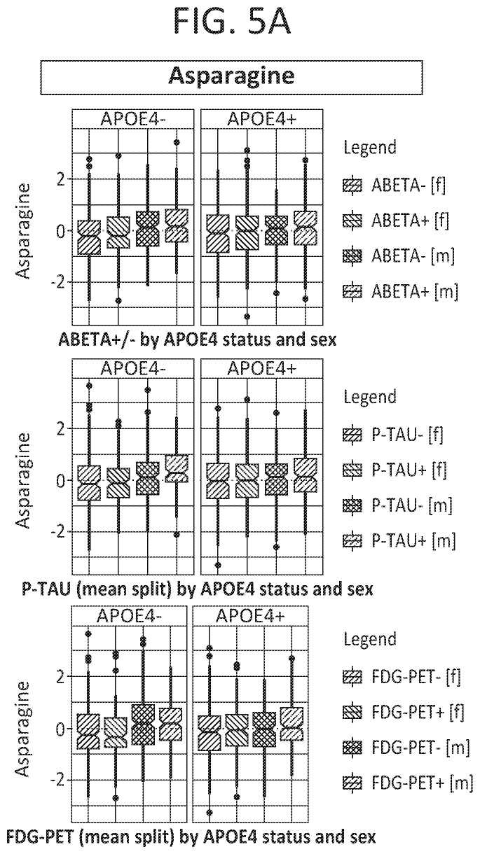

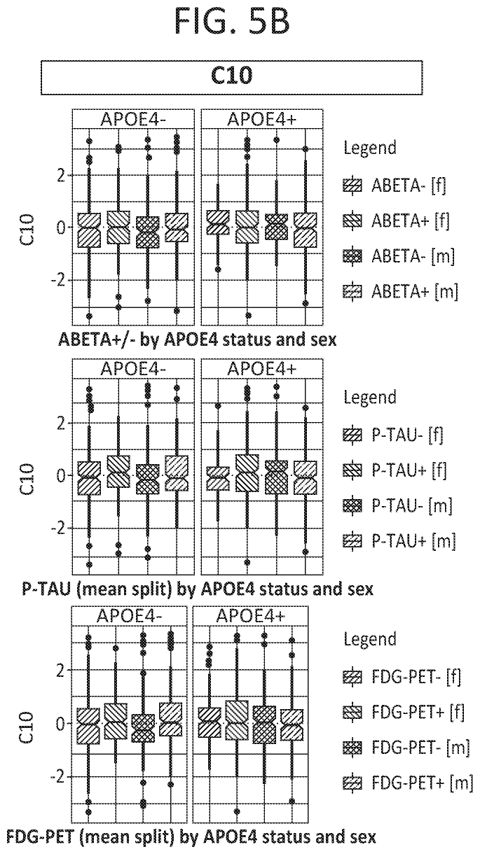

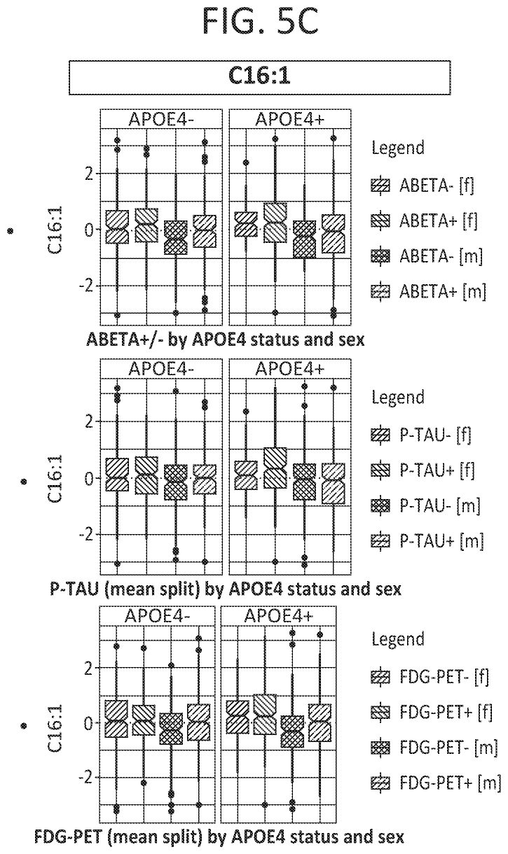

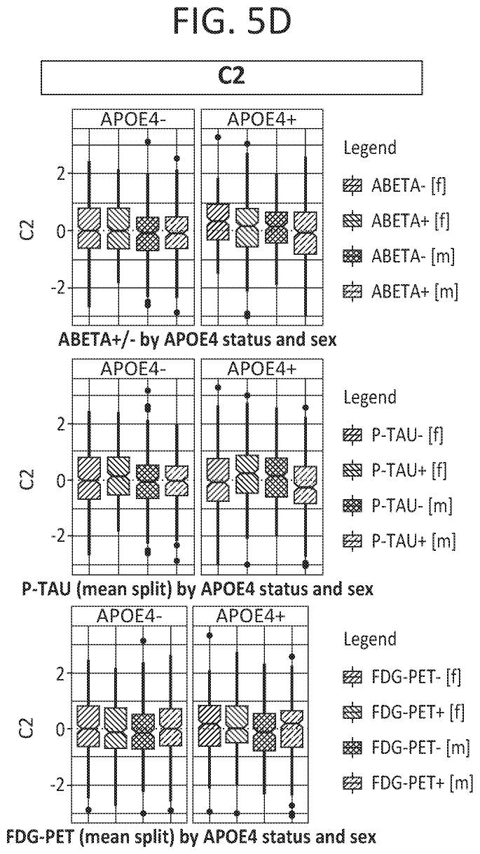

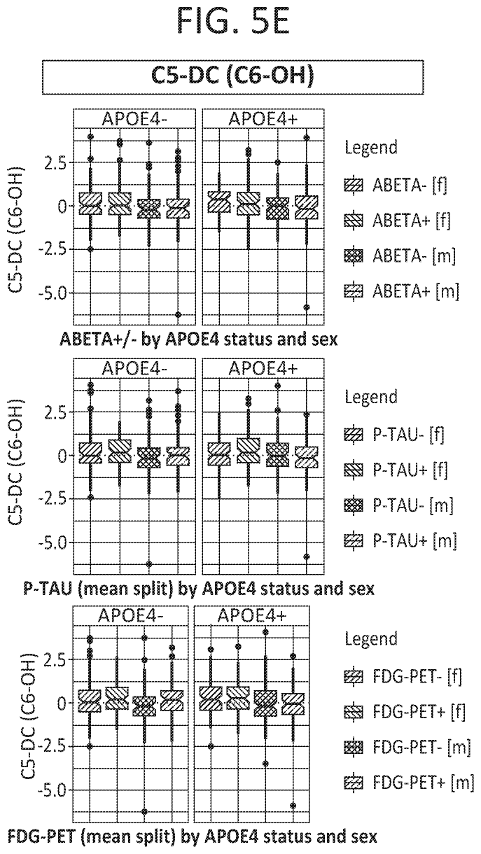

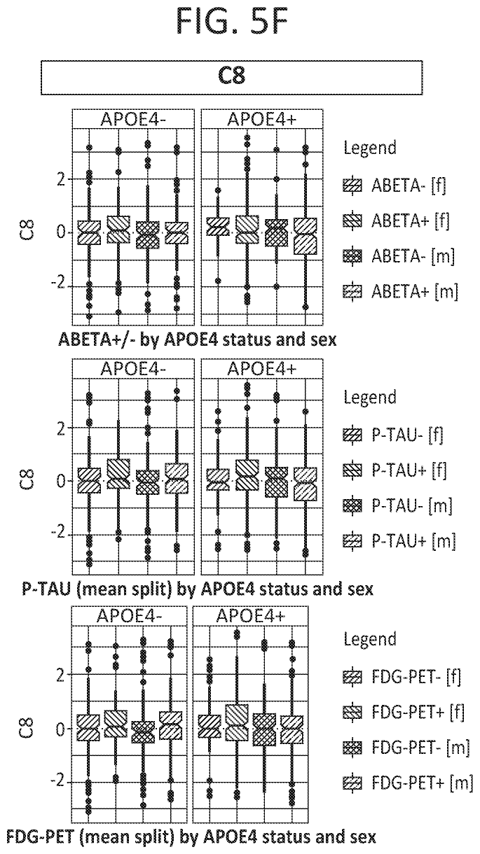

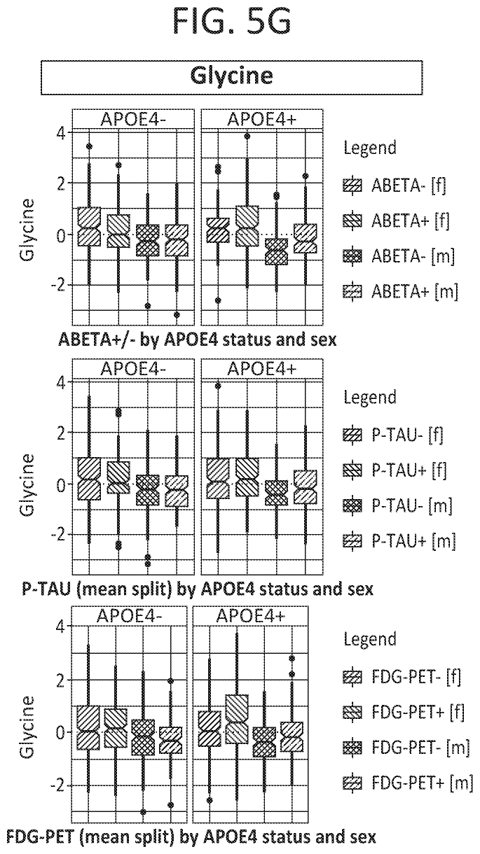

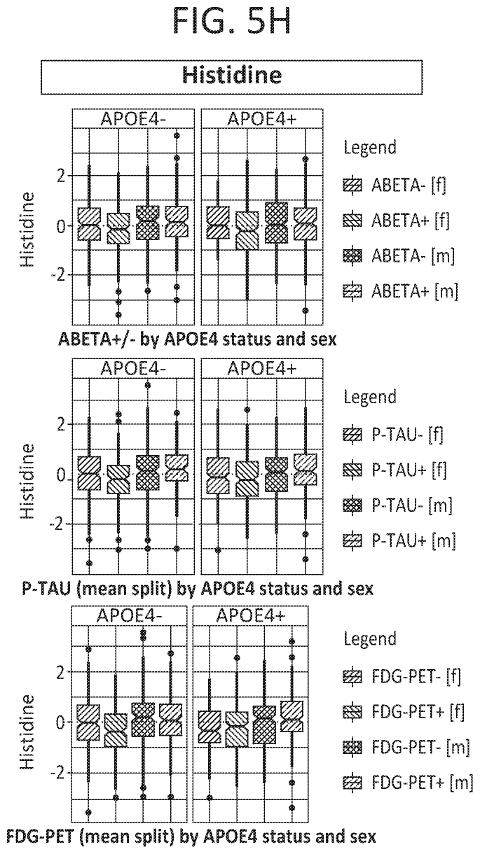

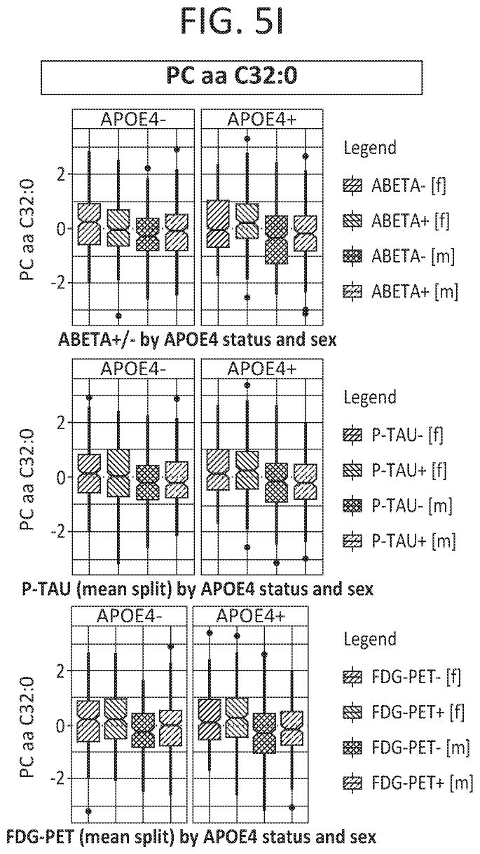

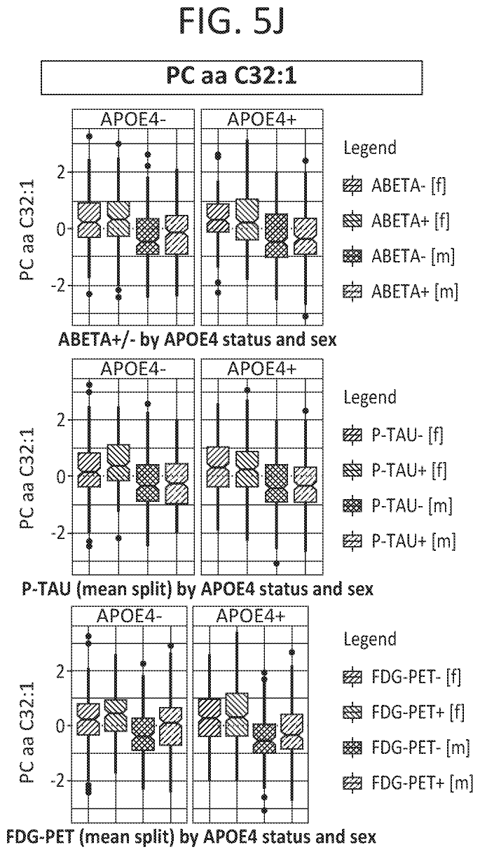

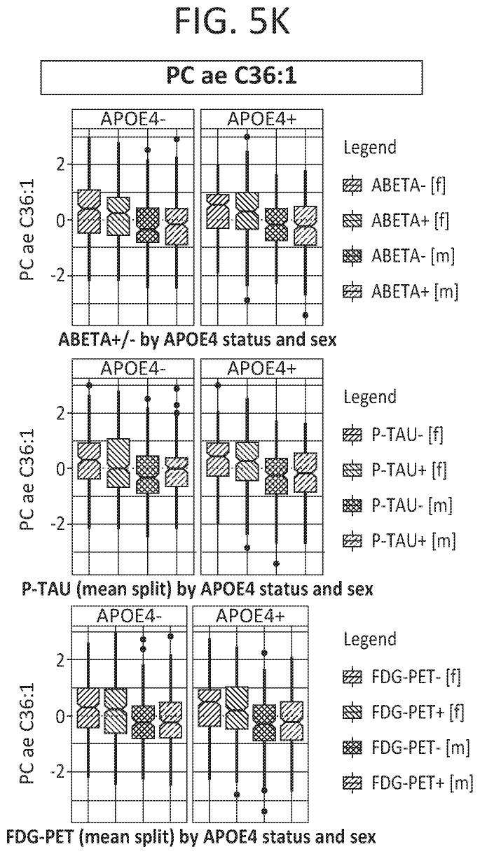

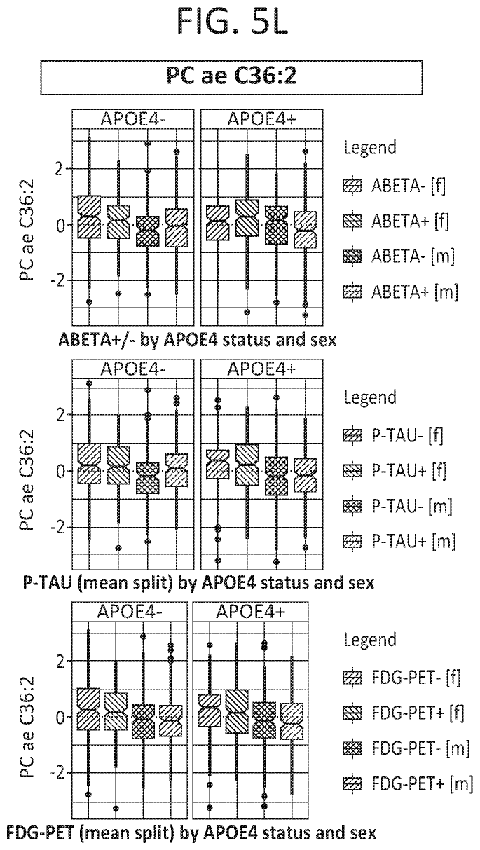

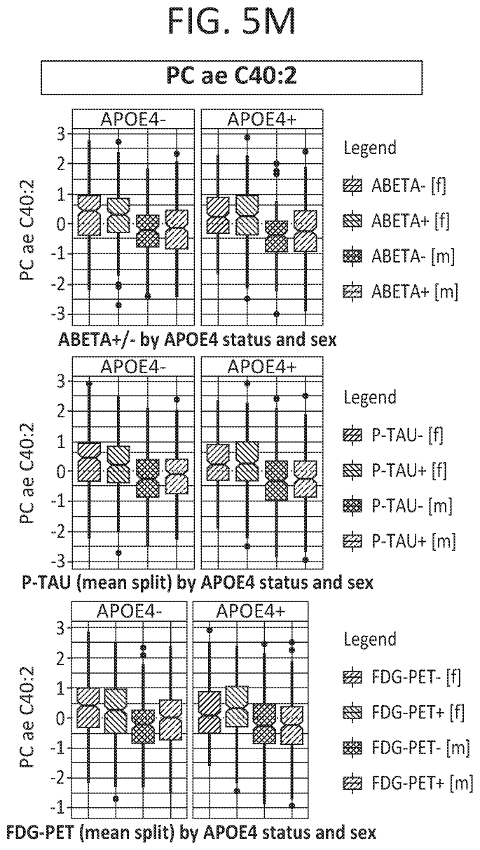

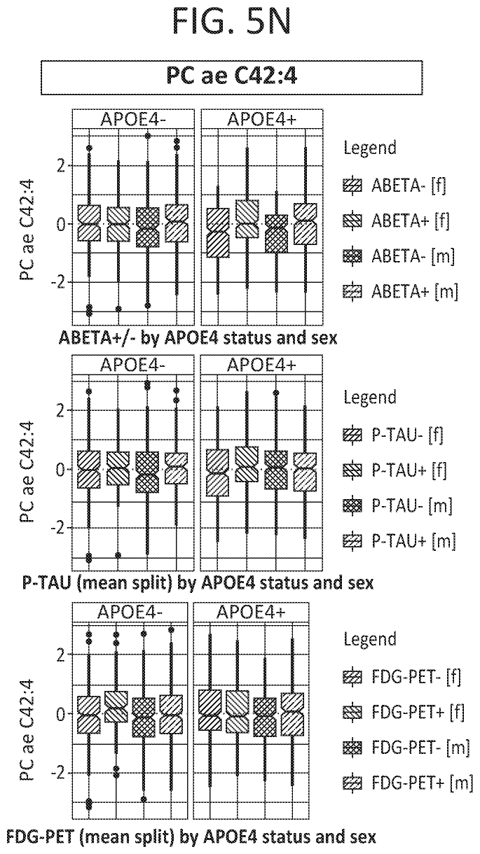

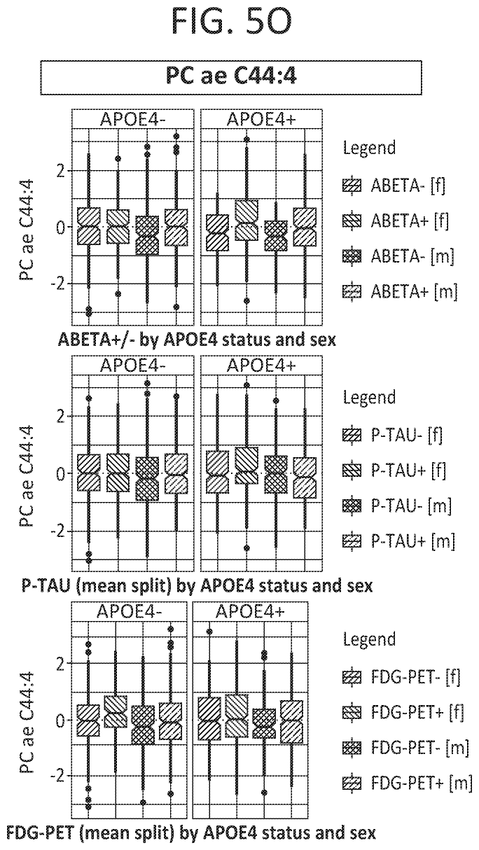

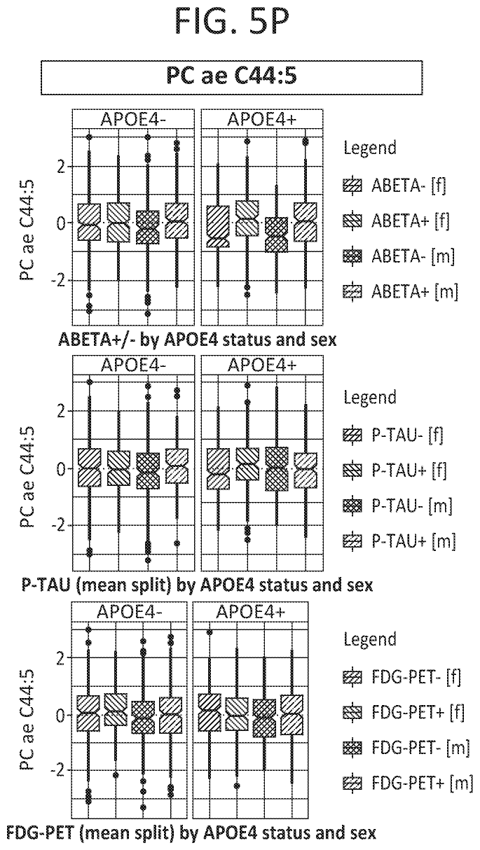

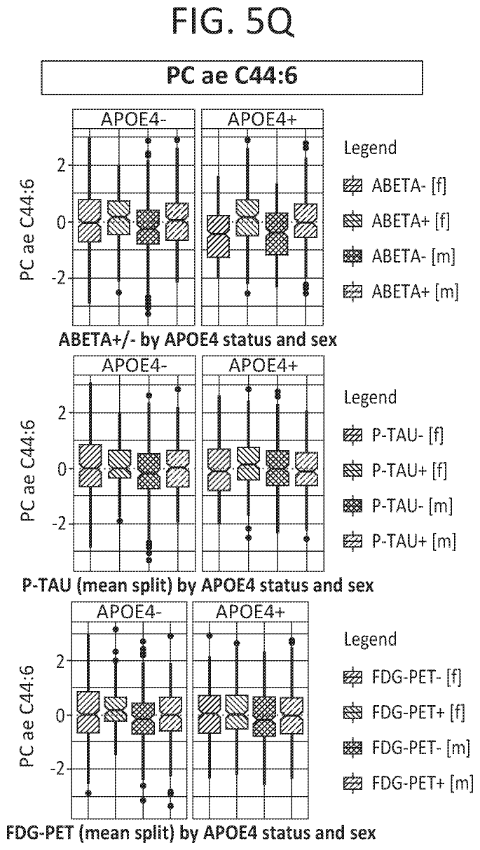

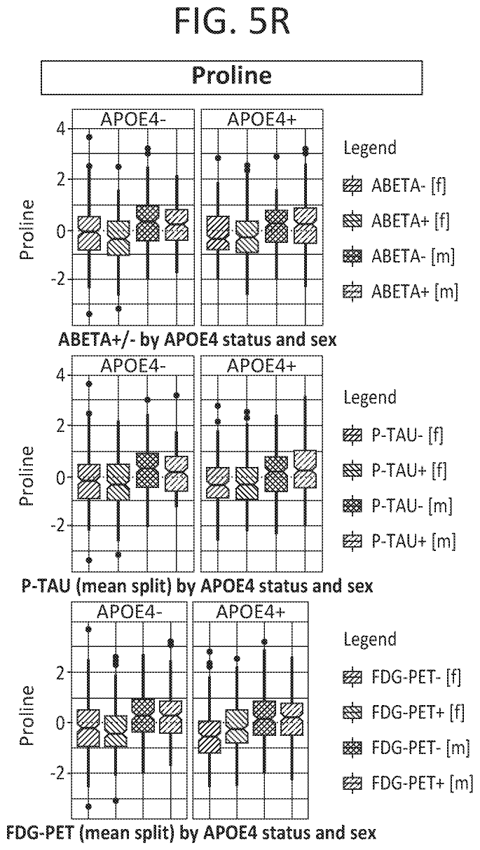

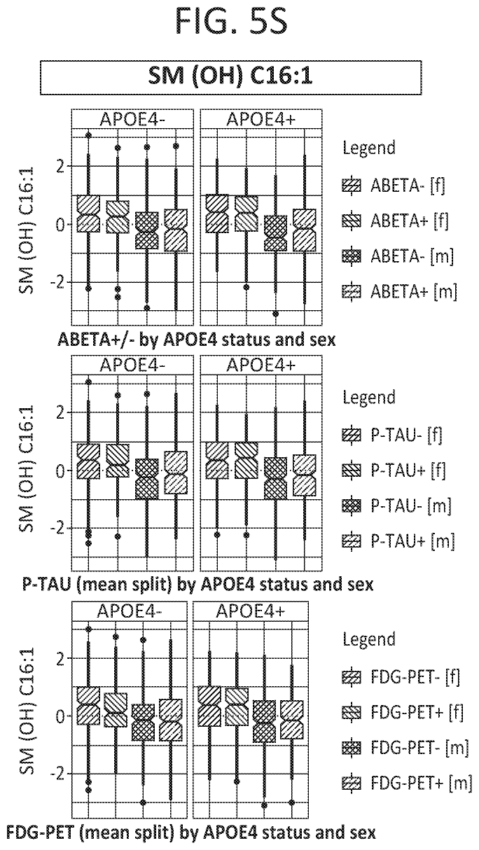

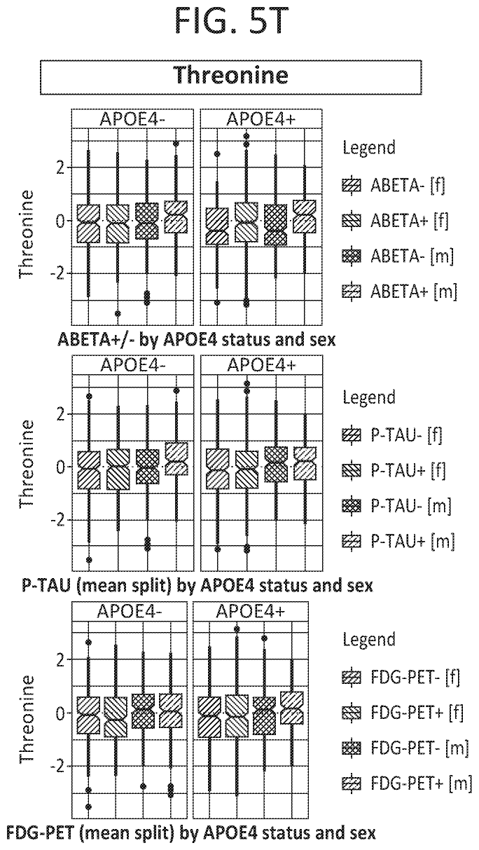

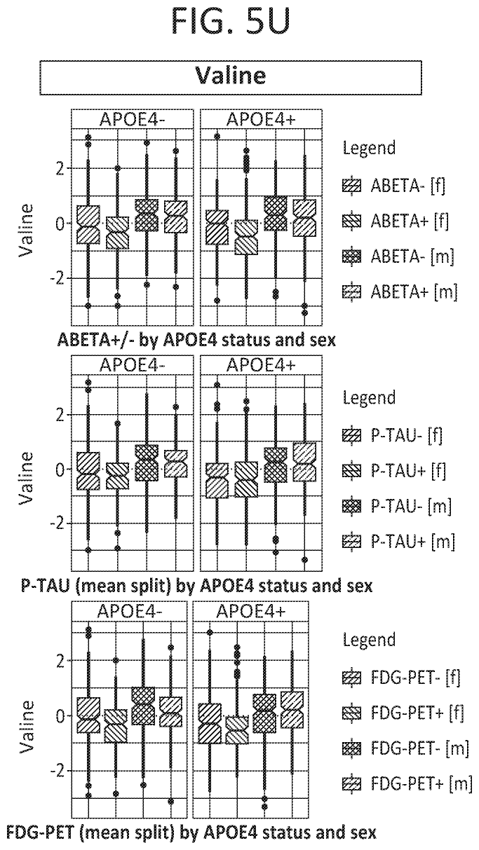

Notably, the full set of metabolites yielding significant effect heterogeneity when comparing APOE ε4-carriers vs. non-carriers (C8, C10, glycine, proline, and valine) also showed significant heterogeneity estimates in the sex-stratified analyses. We therefore applied two-fold stratification by sex and APOE4 status to identify potential interactions between both variables (FIG. 5A-U). This analysis revealed several associations that showed Bonferroni significance in the group with presumably the highest AD risk, namely APOE4+ females. One of those, the significant association of higher proline levels with reduced brain glucose uptake, was not observed in any of the three other strata, in the one-fold stratifications, or in the full sample, emphasizing the value of more fine-granular stratified analyses as proposed here.

The heterogeneity of metabolite effects identified in our study might, in part, explain inconsistencies (e.g., [74] vs. [75]) in associations of metabolites and AD reported in different studies (e.g., if sex and APOE genotype are distributed differently and sample sizes are small). Besides the heterogeneous, sex-specific effects observed for metabolite associations with CSF Aβ₁₋₄₂ and FDG-PET biomarkers, we also found associations of these biomarkers with metabolites that showed the same effects in women and men. In particular, phosphatidylcholines that presumably contain two long-chain fatty acids with, in total, 4 or 5 double bonds (PC ae C44:4, PC ae C44:5) were significant for both AD biomarkers. Such homogeneous metabolite associations would be expected to replicate well across studies.

To test this assumption in an independent sample, we performed a targeted analysis using the three PCs associated with CSF Aβ₁₋₄₂ pathology in 86 serum samples of subjects in the ROS/MAP cohorts: all three associations were Bonferroni significant (PC ae C44:4—P=3.73×10⁻³; PC ae C44:5—P=1.15×10⁻²; PC ae C44:6—P=3.28×10⁻³) in ROS/MAP with consistent effect directions. Of note, in ROS/MAP, we used a different measure of amyloid pathology (total amyloid load in the brain), which is known to be inversely correlated with CSF Aβ₁₋₄₂ levels [76]. This inverse relationship was mirrored by metabolite effect estimates. These results provide evidence for homogeneous associations to be relevant across cohorts.

We were able to show that for the majority of the non-homogeneous findings reported (60%), the interaction term between metabolite levels and sex were also significant in the pooled analysis. When stratifying by APOE4 status, this was true for an even higher fraction of cases (72.7%). This provides an additional line of support for the conclusions drawn in this work.

Effect heterogeneity between subgroups linked to energy metabolism as reported in this study has several important implications for AD research. First, this heterogeneity could explain inconsistencies of metabolomics findings between studies as observed for AD if participants showed different distributions of variables such as sex and APOE ε4 genotype. Second, pooled analysis with model adjustment for such variables as typically applied for sex can mask substantial effects that are relevant for only a subgroup of people. This is also true for combinations of stratifying variables as we demonstrated for the association of proline with brain glucose uptake in female APOE e4 carriers. As a consequence, drug trials may be more successful if acknowledging between-group differences and targeting the subgroup with the presumably largest benefit in their inclusion criteria. For energy metabolism in particular, group-specific dietary interventions precisely targeting the respective dysfunctional pathways may pose a promising alternative to de novo drug development.

1. A method for stratifying Alzheimer's disease among male and female subjects, the method comprising:
determining in a sample from the subject the level of at least one biomarker metabolite selected from the group consisting of PC ae C44:4, PC ae C44:5, PA ae C44:6, PC aa C32:1, PC aa C32:0, and PC ae C42:4; and
diagnosing the subject as having Alzheimer's disease or an increased risk of Alzheimer's disease when the level of the at least one biomarker metabolite in the sample from the subject is different from or greater than the level in a control.

2. The method of claim 2, wherein the biomarker metabolites are selected from PC ae C44:4, PC ae C44:5, and PA ae C44:6.

3. The method of claim 2, wherein the biomarker metabolites are selected from PC ae C44:4, PC ae C44:5, PC aa C32:1, PC aa C32:0, and PC ae C42:4.

4. A method of diagnosing or detecting Alzheimer's disease in a male subject, the method comprising:
determining in a sample from the subject the level of at least one biomarker metabolite selected from the group consisting of PC ae C32:1, threonine, PC ae C36:1, PC ae C36:2, asparagine, glycine, one hydroxy-SM (SM (OH) C16:1), PC ae C40:2, and C16:1; and
diagnosing the subject as having Alzheimer's disease or an increased risk of Alzheimer's disease when the level of the at least one biomarker metabolite in the sample from the subject is different from or greater than the level in a control.

5. The method of claim 4, wherein the biomarker metabolites comprise PC ae C32:1.

6. The method of claim 4, wherein the biomarker metabolites comprise threonine.

7. The method of claim 4, wherein the biomarker metabolites are selected from PC ae C36:1, PC ae C36:2, asparagine, glycine, and one hydroxy-SM (SM (OH) C16:1).

8. The method of claim 4, wherein the biomarker metabolites are selected from PC ae C40:2 and C16:1.

9. The method of claim 4, wherein the biomarker metabolites comprise C16:1.

10. A method of diagnosing or detecting Alzheimer's disease in a female subject, the method comprising:
determining in a sample from the subject the level of at least one biomarker metabolite selected from the group consisting of C5-DC (C6-OH), C8, C10, C2, valine, proline, and histidine; and
diagnosing the subject as having Alzheimer's disease or an increased risk of Alzheimer's disease when the level of the at least one biomarker metabolite in the sample from the subject is different from or greater than the level in a control.

11. The method of claim 10, wherein the biomarker metabolites comprise valine.

12. The method of claim 10, wherein the biomarker metabolites are selected from C5-DC (C6-OH), C8, C10, C2, and histidine.

13. The method of claim 10, wherein the biomarker metabolites comprise proline.

14. The method of claim 10, wherein the biomarker metabolites comprise C10.

15. A method of diagnosing or detecting Alzheimer's disease in an APOE ε4 carrier subject, the method comprising:
determining in a sample from the subject the level of at least one biomarker metabolite selected from the group consisting of PC ae C44:6, PC ae C44:4, PC ae C44:5, and PC ae C42:4; and
diagnosing the subject as having Alzheimer's disease or an increased risk of Alzheimer's disease when the level of the at least one biomarker metabolite in the sample from the subject is different from or greater than the level in a control.

16. A method of diagnosing or detecting Alzheimer's disease in an APOE ε4 non-carrier subject, the method comprising:
determining in a sample from the subject the level of the biomarker metabolite C10; and
diagnosing the subject as having Alzheimer's disease or an increased risk of Alzheimer's disease when the level of the biomarker metabolite in the sample from the subject is different from or greater than the level in a control.

17. A method of diagnosing or detecting Alzheimer's disease in a female APOE ε4 carrier subject, the method comprising:
determining in a sample from the subject the level of at least one biomarker metabolite selected from PC ae C42:4, PC ae C44:5, PC ae C44:6, C10, and proline; and
diagnosing the subject as having Alzheimer's disease or an increased risk of Alzheimer's disease when the level of the at least one biomarker metabolite in the sample from the subject is different from or greater than the level in a control.

18. The method of claim 1, wherein the sample from the subject comprises whole blood, serum, plasma, or cerebral spinal fluid (CSF).

19. The method of claim 1, the method further comprising administering to the subject a treatment for Alzheimer's disease.

20. The method of claim 1, wherein the control sample is taken from a subject or population of subjects with normal cognition.
